(19) United States
(12) Patent Application Publication
(10) Pub. No.: US 2023/0167122 A1
(43) Pub. Date: Jun. 1, 2023

(54) PESTICIDALLY ACTIVE HETEROCYCLIC DERIVATIVES WITH SULFUR CONTAINING SUBSTITUENTS

(71) Applicant: SYNGENTA CROP PROTECTION AG, Basel (CH)

(72) Inventors: Sebastian RENDLER, Basel (CH); Andrew EDMUNDS, Stein (CH); Vikas SIKERVAR, Bracknell, Berkshire (GB); Michel MUEHLEBACH, Stein (CH); André STOLLER, Stein (CH); Daniel EMERY, Stein (CH); Benedikt KURTZ, Stein (CH)

(73) Assignee: SYNGENTA CROP PROTECTION AG, Basel (CH)

(21) Appl. No.: 17/997,441

(22) PCT Filed: Apr. 29, 2021
(86) PCT No.: PCT/EP2021/061315

§ 371 Date: Oct. 28, 2022

(30) Foreign Application Priority Data
Apr. 30, 2020 (IN) 202011018548
Mar. 3, 2021 (IN) 202111008931

(51) Int. Cl. C07D 487/04; C07D 471/04; C07D 401/04; C07D 491/056; C07D 498/04; A01N 43/90; A01N 43/40

(52) U.S. Cl. CPC C07D 487/04; C07D 471/04; C07D 401/04; C07D 491/056; C07D 498/04; A01N 43/90; A01N 43/40

(57) ABSTRACT

Compounds of the formula (I) are disclosed wherein the substituents are as defined in claim 1. Furthermore, the present invention relates to agrochemical compositions which comprise compounds of formula (I), to preparation of these compositions, and to the use of the compounds or compositions in agriculture or horticulture for combating, preventing or controlling animal pests, including arthropods and in particular insects or representatives of the order Acarina.

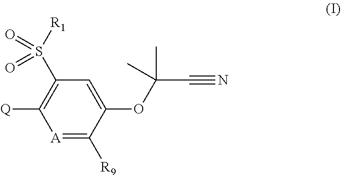

The present invention relates to pesticidally active, in particular insecticidally active heterocyclic derivatives containing sulfur substituents, to processes for their preparation, to compositions comprising those compounds, and to their use for controlling animal pests, including arthropods and in particular insects or representatives of the order Acarina.

Pesticidally active heterocyclic derivatives containing sulfur substituents have previously been described in the literature, for example, in WO12/086848, WO13/018928, WO15/000715, WO15/121136, WO18/197315, WO18/206348, JP2019/081800, and WO19/065568.

It has now surprisingly been found that certain novel sulfur-containing phenyl and pyridyl derivatives with a cyanoisopropoxy group have favorable properties as pesticides.

The present invention therefore provides compounds of formula I,

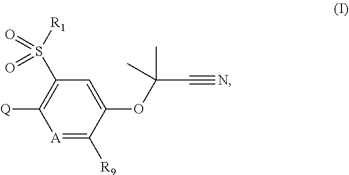

wherein

A is CH or N;

R₁ is C₁-C₄ alkyl or C₃-C₆cycloalkyl-C₁-C₄alkyl;

R₉ is hydrogen or C₁-C₄alkyl;

Q is a radical selected from the group consisting of formula Q₁ to Q₇

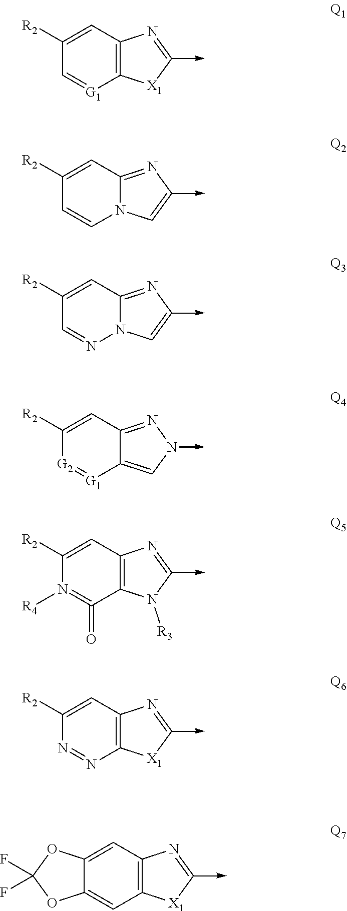

wherein the arrow denotes the point of attachment to the ring incorporating the radical A;

and wherein

X₁ is O, S or NR₃;

R₃ is C₁-C₄alkyl;

R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;

G₁ and G₂ are, independently from each other, N or CH;

R₄ is C₁-C₄alkyl, C₁-C₄haloalkyl, C₃-C₆cycloalkyl or C₁-C₄alkoxy; or

an agrochemically acceptable salt, stereoisomer, enantiomer, tautomer or N-oxide of a compound of formula I.

Compounds of formula I which have at least one basic centre can form, for example, acid addition salts, for example with strong inorganic acids such as mineral acids, for example perchloric acid, sulfuric acid, nitric acid, nitrose acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl and their branched isomers. Alkylsulfanyl, alkylsulfinyl, alkylsulfonyl and alkoxy radicals are derived from the alkyl radicals mentioned.

Halogen is generally fluorine, chlorine, bromine or iodine. This also applies, correspondingly, to halogen in combination with other meanings, such as haloalkyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals; preferably methoxy and ethoxy.

Alkoxyalkyl groups preferably have a chain length of 1 to 6 carbon atoms, more preferably a chain length of 1 to 4 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Alkylsulfanyl is for example methylsulfanyl, ethylsulfanyl, propylsulfanyl, isopropylsulfanyl, butylsulfanyl, pentylsulfanyl, and hexylsulfanyl.

Alkylsulfinyl is for example methylsulfinyl, ethylsulfinyl, propylsulfinyl, isopropylsulfinyl, a butylsulfinyl, pentylsulfinyl, and hexylsulfinyl.

Alkylsulfonyl is for example methylsulfonyl, ethylsulfonyl, propylsulfonyl, isopropylsulfonyl, butylsulfonyl, pentylsulfonyl, and hexylsulfonyl.

The cycloalkyl groups preferably have from 3 to 6 ring carbon atoms, for example cyclopropyl, cyclobutyl, cyclopentyl and cyclohexyl.

Haloalkylsulfanyl groups preferably have a chain length of from 1 to 4 carbon atoms. Haloalkylsulfanyl is, for example, difluoromethylsulfanyl, trifluoromethylsulfanyl or 2,2,2-trifluoroethylsulfanyl. Similar considerations apply to the radicals C₁-C₄haloalkylsulfinyl and C₁-C₄haloalkylsulfonyl, which may be, for example, trifluoromethylsulfinyl, trifluoromethylsulfonyl or 2,2,2-trifluoroethylsulfonyl.

The compounds of formula I according to the invention also include hydrates which may be formed during the salt formation.

Embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein:

A is CH or N;

R₁ is ethyl, propyl, isopropyl or —CH₂cyclopropyl;

R₉ is hydrogen, methyl or ethyl.

Embodiment 3a provides compounds, or an agrochemically acceptable salt, stereoisomer, enantiomer, tautomer or N-oxide thereof, according to embodiment 1 wherein:

A is CH or N;

R₁ is ethyl or —CH₂cyclopropyl; preferably R₁ is ethyl; and

R₉ is hydrogen or methyl.

Embodiment 3b provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl; and

R₉ is hydrogen or methyl.

Embodiment 4a provides compounds, or an agrochemically acceptable salt, stereoisomer, enantiomer, tautomer or N-oxide thereof, according to embodiment 1 wherein:

A is CH or N;

R₁ is ethyl or —CH₂cyclopropyl; preferably R₁ is ethyl; and

R₉ is hydrogen.

Embodiment 4b provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl; and

R₉ is hydrogen.

Embodiment 5a provides compounds, or an agrochemically acceptable salt, stereoisomer, enantiomer, tautomer or N-oxide thereof, according to embodiment 1 wherein:

A is CH or N;

R₁ is ethyl or —CH₂cyclopropyl; preferably R₁ is ethyl; and

R₉ is methyl.

Embodiment 5b provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl; and

R₉ is methyl.

Embodiment 6 provides compounds, or an agrochemically acceptable salt, stereoisomer, enantiomer, tautomer or N-oxide thereof, according to embodiment 1 wherein:

Q is a radical selected from Q₁, Q₂, Q₄ and Q₅

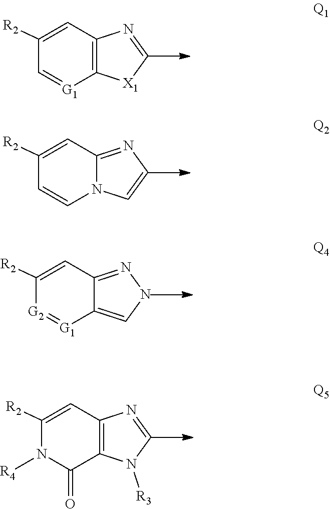

wherein the arrow denotes the point of attachment to the ring incorporating the radical A;

and wherein R₂ is C₁-C₂haloalkyl, C₁-C₂haloalkylsulfanyl, C₁-C₂haloalkylsulfinyl or C₁-C₂haloalkylsulfonyl;

X₁ is oxygen or NCH₃;

R₃ is C₁-C₂alkyl;

R₄ is C₁-C₂alkyl, C₁-C₂haloalkyl, C₁-C₂alkoxy or cyclopropyl; and

G₁ and G₂ are, independently from each other, N or CH.

Embodiment 7 provides compounds, or an agrochemically acceptable salt, stereoisomer, enantiomer, tautomer or N-oxide thereof, according to embodiment 1 wherein:

Q is a radical selected from Q₁, Q₂ and Q₅

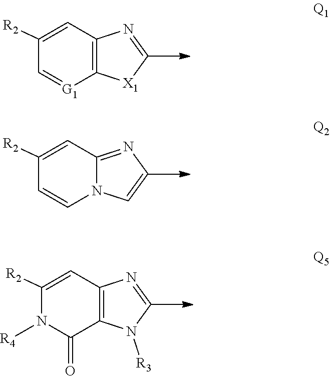

wherein the arrow denotes the point of attachment to the ring incorporating the radical A;

and wherein

R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;

X₁ is NCH₃;

R₃ is methyl;

R₄ is methyl, ethyl, 2,2,2-trifluoroethyl, methoxy or cyclopropyl; and

G₁ is N or CH.

Embodiment 8a provides compounds, or an agrochemically acceptable salt, stereoisomer, enantiomer, tautomer or N-oxide thereof, according to embodiment 1 wherein:

Q is a radical selected from Q₁, Q₂ and Q₅

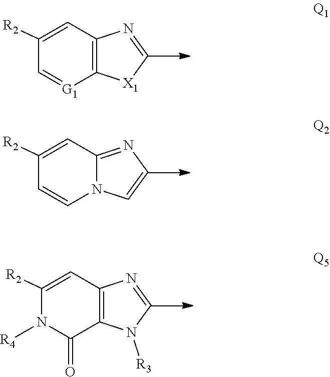

wherein the arrow denotes the point of attachment to the ring incorporating the radical A;

and wherein

R₂ is trifluoromethyl, pentafluoroethyl, trifluoromethylsulfanyl, trifluoromethylsulfinyl or trifluoromethylsulfonyl;

X₁ is NCH₃;

R₃ is methyl;

R₄ is ethyl, methoxy or cyclopropyl; and

G₁ is CH or N.

Embodiment 8b provides compounds, or an agrochemically acceptable salt, stereoisomer, enantiomer, tautomer or N-oxide thereof, according to embodiment 1 wherein:

Q is a radical selected from Q₁, Q₂ and Q₅

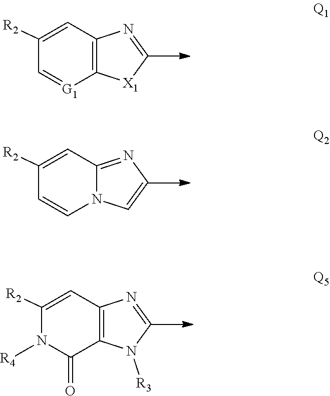

wherein the arrow denotes the point of attachment to the ring incorporating the radical A;

and wherein

R₂ is trifluoromethyl, pentafluoroethyl or trifluoromethylsulfanyl;

X₁ is NCH₃;

R₃ is methyl;

R₄ is ethyl or cyclopropyl; and

G₁ is CH or N.

Embodiment 8c provides compounds, or an agrochemically acceptable salt, stereoisomer, enantiomer, tautomer or N-oxide thereof, according to embodiment 1 wherein:

Q is a radical selected from Q₁, Q₂, Q₅ and Q₇

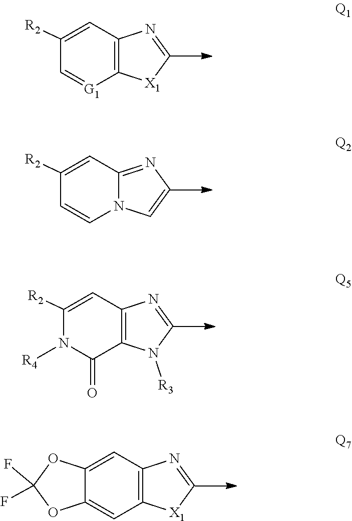

wherein the arrow denotes the point of attachment to the ring incorporating the radical A;

and wherein

R₂ is trifluoromethyl, pentafluoroethyl or trifluoromethylsulfanyl;

X₁ is O or NCH₃;

R₃ is methyl;

R₄ is ethyl or cyclopropyl; and

G₁ is CH or N.

Embodiment 8d provides compounds, or an agrochemically acceptable salt, stereoisomer, enantiomer, tautomer or N-oxide thereof, according to embodiment 1 wherein:

Q is a radical selected from Q₁, Q₂, Q₅ and Q₇

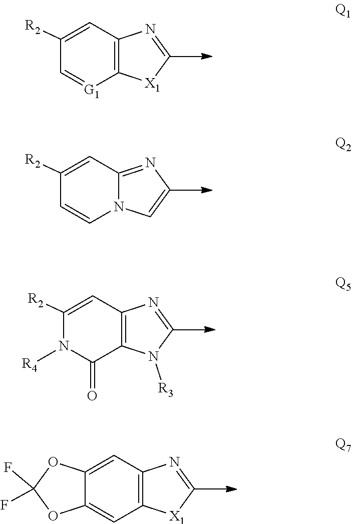

wherein the arrow denotes the point of attachment to the ring incorporating the radical A;

and wherein

R₂ is trifluoromethyl, pentafluoroethyl or trifluoromethylsulfanyl;

X₁ is NCH₃;

R₃ is methyl;

R₄ is ethyl or cyclopropyl; and

G₁ is CH or N.

Embodiment 9 provides compounds, or an agrochemically acceptable salt, stereoisomer, enantiomer, tautomer or N-oxide thereof, according to embodiment 1 wherein:

Q is radical Q₁

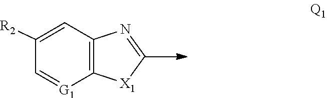

wherein the arrow denotes the point of attachment to the ring incorporating the radical A;

and wherein

R₂ is trifluoromethyl;

X₁ is NCH₃; and

G₁ is CH or N.

Embodiment 10a provides compounds, or an agrochemically acceptable salt, stereoisomer, enantiomer, tautomer or N-oxide thereof, according to embodiment 1 wherein:

Q is radical Q₂

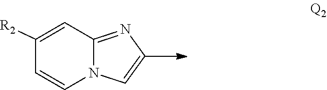

wherein the arrow denotes the point of attachment to the ring incorporating the radical A;

and wherein

R₂ is trifluoromethyl, pentafluoroethyl, trifluoromethylsulfanyl, trifluoromethylsulfinyl or trifluoromethylsulfonyl.

Embodiment 10b provides compounds, or an agrochemically acceptable salt, stereoisomer, enantiomer, tautomer or N-oxide thereof, according to embodiment 1 wherein:

Q is radical Q₂

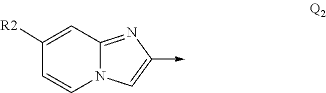

wherein the arrow denotes the point of attachment to the ring incorporating the radical A;

and wherein

R₂ is trifluoromethyl, pentafluoroethyl or trifluoromethylsulfanyl.

Embodiment 11 provides compounds, or an agrochemically acceptable salt, stereoisomer, enantiomer, tautomer or N-oxide thereof, according to embodiment 1 wherein:

Q is radical Q₅

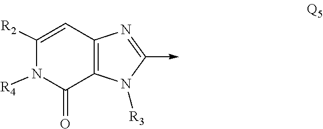

wherein the arrow denotes the point of attachment to the ring incorporating the radical A;

and wherein

R₂ is trifluoromethyl;

R₃ is methyl; and

R₄ is ethyl or cyclopropyl.

Embodiment 11a provides compounds, or an agrochemically acceptable salt, stereoisomer, enantiomer, tautomer or N-oxide thereof, according to embodiment 1 wherein:

Q is radical Q₇

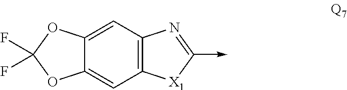

wherein the arrow denotes the point of attachment to the ring incorporating the radical A;

and wherein

X₁ is O or NCH₃.

Embodiment 12 provides compounds, or an agrochemically acceptable salt, stereoisomer, enantiomer, tautomer or N-oxide thereof, according to embodiment 1 wherein:

A is CH or N;

R₁ is ethyl, propyl, isopropyl or —CH₂cyclopropyl;

R₉ is hydrogen, methyl or ethyl;

Q is a radical selected from Q₁, Q₂, Q₄ and Q₅

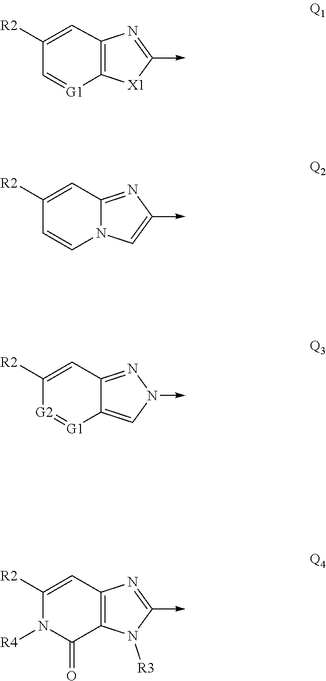

wherein the arrow denotes the point of attachment to the ring incorporating the radical A;

and wherein

R₂ is C₁-C₂haloalkyl, C₁-C₂haloalkylsulfanyl, C₁-C₂haloalkylsulfinyl or C₁-C₂haloalkylsulfonyl;

X₁ is oxygen or NCH₃;

R₃ is C₁-C₂alkyl;

R₄ is C₁-C₂alkyl, C₁-C₂haloalkyl, C₁-C₂alkoxy or cyclopropyl; and

G₁ and G₂ are, independently from each other, N or CH.

Embodiment 13 provides compounds, or an agrochemically acceptable salt, stereoisomer, enantiomer, tautomer or N-oxide thereof, according to embodiment 1 wherein:

A is CH or N;

R₁ is ethyl or —CH₂cyclopropyl; preferably R₁ is ethyl;

R₉ is hydrogen or methyl;

Q is a radical selected from Q₁, Q₂ and Q₅

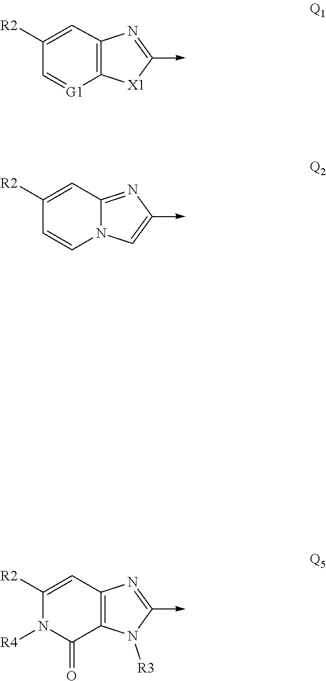

wherein the arrow denotes the point of attachment to the ring incorporating the radical A;

and wherein

R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;

X₁ is NCH₃;

R₃ is methyl;

R₄ is methyl, ethyl, 2,2,2-trifluoroethyl, methoxy or cyclopropyl; and

G₁ is N or CH.

Embodiment 14 provides compounds, or an agrochemically acceptable salt, stereoisomer, enantiomer, tautomer or N-oxide thereof, according to embodiment 1 wherein:

A is CH or N;

R₁ is or —CH₂cyclopropyl; preferably R₁ is ethyl;

R₉ is hydrogen or methyl;

Q is a radical selected from Q₁, Q₂ and Q₅

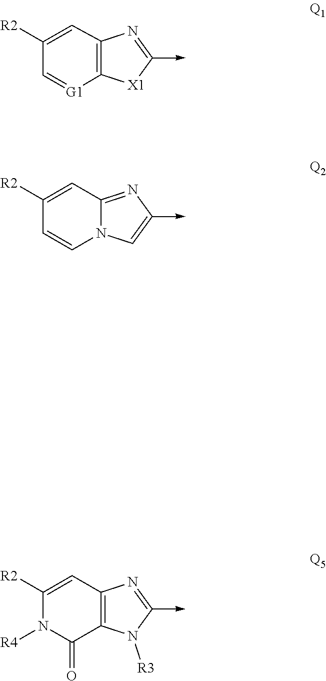

wherein the arrow denotes the point of attachment to the ring incorporating the radical A;

and wherein

R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;

X₁ is NCH₃;

R₃ is methyl;

R₄ is methyl, ethyl, 2,2,2-trifluoroethyl, methoxy or cyclopropyl; and

G₁ is N or CH.

Embodiment 15 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl;

R₉ is hydrogen or methyl;

Q is a radical selected from Q₁, Q₂ and Q₅

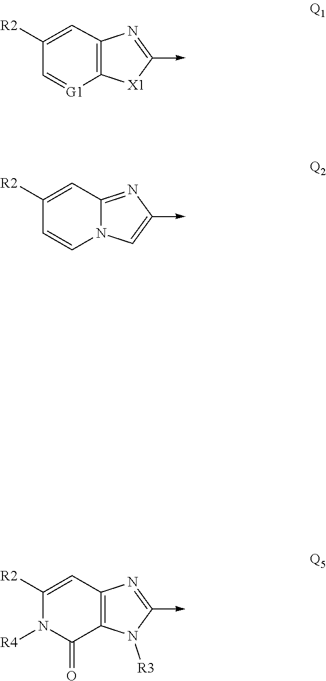

wherein the arrow denotes the point of attachment to the ring incorporating the radical A;

and wherein

R₂ is trifluoromethyl, pentafluoroethyl, trifluoromethylsulfanyl, trifluoromethylsulfinyl or trifluoromethylsulfonyl;

X₁ is NCH₃;

R₃ is methyl;

R₄ is ethyl, methoxy or cyclopropyl; and

G₁ is CH or N.

Embodiment 16 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl R₉ is hydrogen or methyl;

Q is a radical selected from Q₁, Q₂ and Q₅

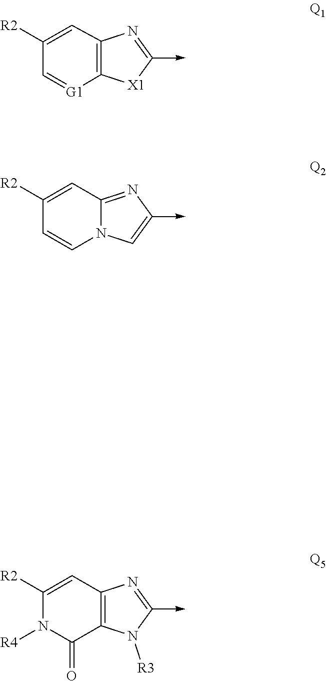

wherein the arrow denotes the point of attachment to the ring incorporating the radical A;

and wherein

R₂ is trifluoromethyl, pentafluoroethyl or trifluoromethylsulfanyl;

X₁ is NCH₃;

R₃ is methyl;

R₄ is ethyl or cyclopropyl; and

G₁ is CH or N.

Embodiment 16a provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl R₉ is hydrogen or methyl;

Q is a radical selected from Q₁, Q₂, Q₅ and Q₇

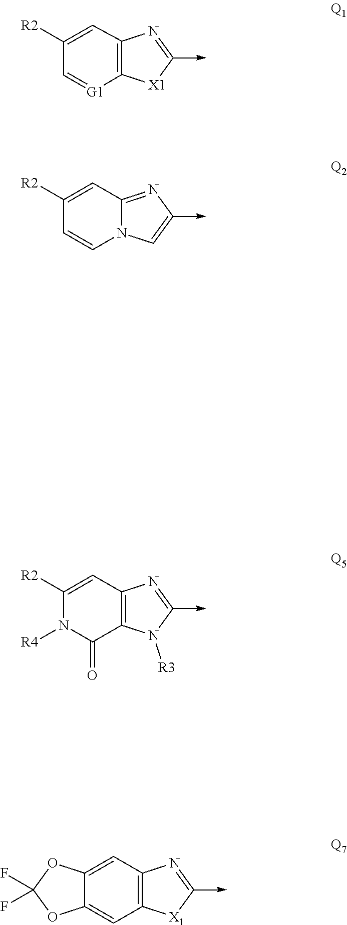

wherein the arrow denotes the point of attachment to the ring incorporating the radical A;

and wherein

R₂ is trifluoromethyl, pentafluoroethyl or trifluoromethylsulfanyl;

X₁ is O or NCH₃;

R₃ is methyl;

R₄ is ethyl or cyclopropyl; and

G₁ is CH or N.

Embodiment 17 provides compounds, or an agrochemically acceptable salt, stereoisomer, enantiomer, tautomer or N-oxide thereof, according to embodiment 1 wherein:

A is N;

R₁ ethyl or —CH₂cyclopropyl; preferably R₁ is ethyl R₉ is hydrogen or methyl;

Q is radical Q₁

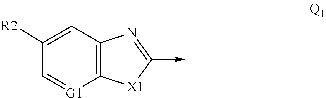

wherein the arrow denotes the point of attachment to the ring incorporating the radical A;

and wherein

R₂ is trifluoromethyl;

X₁ is NCH₃; and

G₁ is N or CH.

Embodiment 18 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl;

R₉ is hydrogen;

Q is radical Q₁

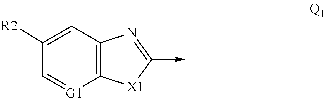

wherein the arrow denotes the point of attachment to the ring incorporating the radical A;

and wherein

R₂ is trifluoromethyl;

X₁ is NCH₃; and

G₁ is N or CH.

Embodiment 19 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl

R₉ is methyl;

Q is radical Q₁

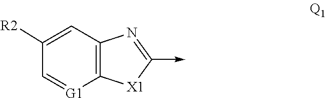

wherein the arrow denotes the point of attachment to the ring incorporating the radical A;

and wherein

R₂ is trifluoromethyl;

X₁ is NCH₃; and

G₁ is N or CH.

Embodiment 20 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl;

R₉ is hydrogen;

Q is radical Q₂

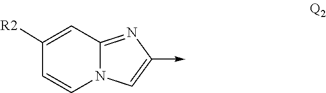

wherein the arrow denotes the point of attachment to the ring incorporating the radical A;

and wherein

R₂ is trifluoromethyl, pentafluoroethyl, trifluoromethylsulfanyl, trifluoromethylsulfinyl or trifluoromethylsulfonyl.

Embodiment 21 provides compounds, or an agrochemically acceptable salt, stereoisomer, enantiomer, tautomer or N-oxide thereof, according to embodiment 1 wherein:

A is N;

R₁ is ethyl or —CH₂cyclopropyl; preferably R₁ is ethyl;

R₉ is hydrogen;

Q is radical Q₂

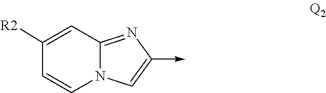

wherein the arrow denotes the point of attachment to the ring incorporating the radical A;

and wherein

R₂ is trifluoromethyl, pentafluoroethyl or trifluoromethylsulfanyl.

Embodiment 22 provides compounds, or an agrochemically acceptable salt, stereoisomer, enantiomer, tautomer or N-oxide thereof, according to embodiment 1 wherein:

A is CH;

R₁ is ethyl or —CH₂cyclopropyl; preferably R₁ is ethyl;

R₉ is hydrogen;

Q is radical Q₂

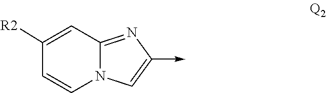

wherein the arrow denotes the point of attachment to the ring incorporating the radical A;

and wherein

R₂ is trifluoromethyl, pentafluoroethyl, trifluoromethylsulfanyl, trifluoromethylsulfinyl or trifluoromethylsulfonyl.

Embodiment 23 provides compounds, or an agrochemically acceptable salt, stereoisomer, enantiomer, tautomer or N-oxide thereof, according to embodiment 1 wherein:

A is CH;

R₁ is ethyl or —CH₂cyclopropyl; preferably R₁ is ethyl;

R₉ is hydrogen;

Q is radical Q₂

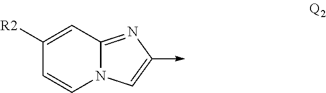

wherein the arrow denotes the point of attachment to the ring incorporating the radical A;

and wherein

R₂ is trifluoromethyl, pentafluoroethyl or trifluoromethylsulfanyl.

Embodiment 24 provides compounds, or an agrochemically acceptable salt, stereoisomer, enantiomer, tautomer or N-oxide thereof, according to embodiment 1 wherein:

A is N;

R₁ is ethyl;

R₉ is hydrogen;

Q is radical Q₅

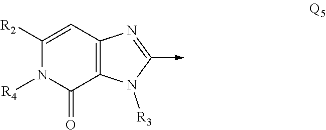

wherein the arrow denotes the point of attachment to the ring incorporating the radical A;

and wherein

R₂ is trifluoromethyl;

R₃ is methyl; and

R₄ is ethyl or cyclopropyl.

Embodiment 25 provides compounds, or an agrochemically acceptable salt, stereoisomer, enantiomer, tautomer or N-oxide thereof, according to embodiment 1 wherein:

A is N;

R₁ is ethyl;

R₉ is hydrogen;

Q is radical Q₇

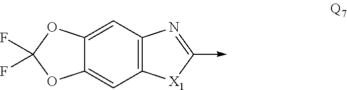

wherein the arrow denotes the point of attachment to the ring incorporating the radical A;

and wherein

X₁ is O or NCH₃.

A preferred group of compounds of formula I is represented by the compounds of formula I-1

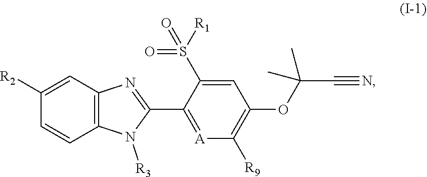

wherein R₁, R₂, R₃, R₉, and A are as defined under formula I above; or

an agrochemically acceptable salt, stereoisomer, enantiomer, tautomer or N-oxide of formula I-1.

In one preferred group of compounds of formula I-1, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl.

In another preferred group of compounds of formula I-1, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen.

In compounds of formula I-1 and all of the preferred embodiments of compounds of formula I-1 mentioned above, unless otherwise specified, R₁, R₂, R₃, R₉, and A are as defined under formula I above; A is CH or N, preferably A is N; R₂ is trifluoromethyl, pentafluoroethyl or trifluoromethylsulfanyl or trifluoromethylsulfonyl; preferably R₂ is trifluoromethyl; R₃ is methyl; and R₉ is hydrogen.

Another preferred group of compounds of formula I is represented by the compounds of formula I-2

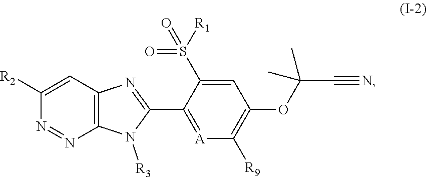

wherein R₁, R₂, R₃, R₉, and A are as defined under formula I above; or

an agrochemically acceptable salt, stereoisomer, enantiomer, tautomer or N-oxide of formula I-2.

In one preferred group of compounds of formula I-2, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl.

In another preferred group of compounds of formula I-2, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen.

In compounds of formula I-2 and all of the preferred embodiments of compounds of formula I-2 mentioned above, unless otherwise specified, R₁, R₂, R₃, R₉, and A are as defined under formula I above; preferably A is CH or N, more preferably A is N; R₂ is trifluoromethyl, pentafluoroethyl or trifluoromethylsulfanyl or trifluoromethylsulfonyl; preferably R₂ is trifluoromethyl; R₃ is methyl; and R₉ is hydrogen.

Another preferred group of compounds of formula I is represented by the compounds of formula I-3

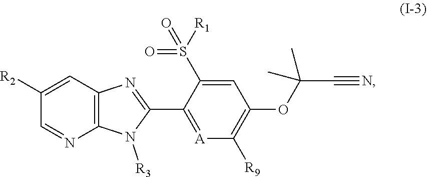

wherein R₁, R₂, R₃, R₉, and A are as defined under formula I above; or

an agrochemically acceptable salt, stereoisomer, enantiomer, tautomer or N-oxide of formula I-3.

In one preferred group of compounds of formula I-3, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl.

In another preferred group of compounds of formula I-3, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen.

In compounds of formula I-3 and all of the preferred embodiments of compounds of formula I-3 mentioned above, unless otherwise specified, R₁, R₂, R₃, R₉, and A are as defined under formula I above; preferably A is CH or N, more preferably A is N; R₂ is trifluoromethyl, pentafluoroethyl or trifluoromethylsulfanyl or trifluoromethylsulfonyl; preferably R₂ is trifluoromethyl; R₃ is methyl; and R₉ is hydrogen.

Another preferred group of compounds of formula I is represented by the compounds of formula I-4

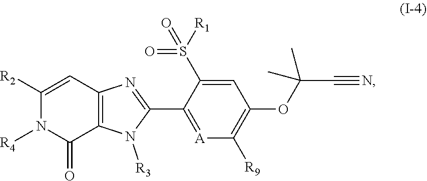

wherein R₁, R₂, R₃, R₄, R₉, and A are as defined under formula I above; or

an agrochemically acceptable salt, stereoisomer, enantiomer, tautomer or N-oxide of formula I-4.

In one preferred group of compounds of formula I-4, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₄ is C₁-C₂alkyl, C₁-C₂haloalkyl, C₁-C₂alkoxy or cyclopropyl; R₉ is hydrogen, methyl or ethyl.

In another preferred group of compounds of formula I-4, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₄ is methyl, ethyl, methoxy or cyclopropyl; R₉ is hydrogen or methyl, preferably R₉ is hydrogen.

In another preferred group of compounds of formula I-4, R₄ is ethyl or cyclopropyl.

In compounds of formula I-4 and all of the preferred embodiments of compounds of formula I-4 mentioned above, unless otherwise specified, R₁, R₂, R₃, R₄, R₉, and A are as defined under formula I above; preferably A is CH or N, more preferably A is N; R₂ is trifluoromethyl, pentafluoroethyl or trifluoromethylsulfanyl or trifluoromethylsulfonyl; preferably R₂ is trifluoromethyl; R₃ is methyl; R₄ is ethyl, methoxy or cyclopropyl; and R₉ is hydrogen.

Another preferred group of compounds of formula I is represented by the compounds of formula I-5

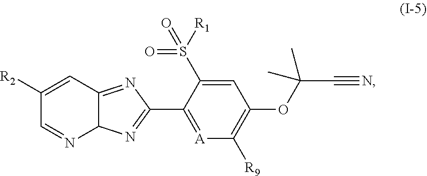

wherein R₁, R₂, R₉, and A are as defined under formula I above; or

an agrochemically acceptable salt, stereoisomer, enantiomer, tautomer or N-oxide of formula I-5.

In one preferred group of compounds of formula I-5, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl.

In another preferred group of compounds of formula I-5, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen.

In compounds of formula I-5 and all of the preferred embodiments of compounds of formula I-5 mentioned above, unless otherwise specified, R₁, R₂, R₉, and A are as defined under formula I above;

preferably A is CH or N, more preferably A is N; R₂ is trifluoromethyl, pentafluoroethyl or trifluoromethylsulfanyl or trifluoromethylsulfonyl; preferably R₂ is trifluoromethyl; and R₉ is hydrogen.

Another preferred group of compounds of formula I is represented by the compounds of formula I-6

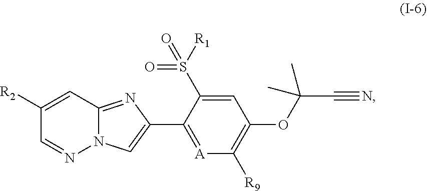

wherein R₁, R₂, R₉, and A are as defined under formula I above; or

an agrochemically acceptable salt, stereoisomer, enantiomer, tautomer or N-oxide of formula I-6.

In one preferred group of compounds of formula I-6, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl.

In another preferred group of compounds of formula I-6, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen.

In compounds of formula I-6 and all of the preferred embodiments of compounds of formula I-6 mentioned above, unless otherwise specified, R₁, R₂, R₉, and A are as defined under formula I above;

preferably A is CH or N, more preferably A is N; R₂ is trifluoromethyl, pentafluoroethyl or trifluoromethylsulfanyl or trifluoromethylsulfonyl; preferably R₂ is trifluoromethyl; and R₉ is hydrogen.

Another preferred group of compounds of formula I is represented by the compounds of formula I-7

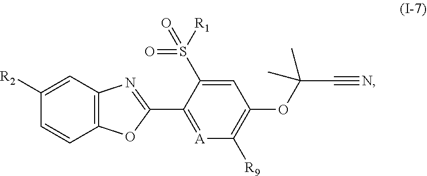

wherein R₁, R₂, R₉, and A are as defined under formula I above; or

an agrochemically acceptable salt, stereoisomer, enantiomer, tautomer or N-oxide of formula I-7.

In one preferred group of compounds of formula I-7, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl.

In another preferred group of compounds of formula I-7, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen.

In compounds of formula I-7 and all of the preferred embodiments of compounds of formula I-7 mentioned above, unless otherwise specified, R₁, R₂, R₉, and A are as defined under formula I above;

preferably A is CH or N, more preferably A is N; R₂ is trifluoromethyl, pentafluoroethyl or trifluoromethylsulfanyl or trifluoromethylsulfonyl; preferably R₂ is trifluoromethyl; and R₉ is hydrogen.

Another preferred group of compounds of formula I is represented by the compounds of formula I-8

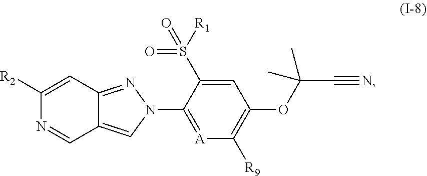

wherein R₁, R₂, R₉, and A are as defined under formula I above; or

an agrochemically acceptable salt, stereoisomer, enantiomer, tautomer or N-oxide of formula I-8.

In one preferred group of compounds of formula I-8, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl.

In another preferred group of compounds of formula I-8, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen.

In compounds of formula I-8 and all of the preferred embodiments of compounds of formula I-8 mentioned above, unless otherwise specified, R₁, R₂, R₉, and A are as defined under formula I above;

preferably A is CH or N, more preferably A is N; R₂ is trifluoromethyl, pentafluoroethyl or trifluoromethylsulfanyl or trifluoromethylsulfonyl; preferably R₂ is trifluoromethyl; and R₉ is hydrogen.

Another preferred group of compounds of formula I is represented by the compounds of formula I-9

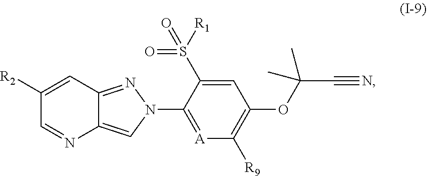

wherein R₁, R₂, R₉, and A are as defined under formula I above; or

an agrochemically acceptable salt, stereoisomer, enantiomer, tautomer or N-oxide of formula I-9.

In one preferred group of compounds of formula I-9, A is CH or N; R₁ is ethyl, propyl or isopropyl or CH₂cyclopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl.

In another preferred group of compounds of formula I-9, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen.

In compounds of formula I-9 and all of the preferred embodiments of compounds of formula I-9 mentioned above, unless otherwise specified, R₁, R₂, R₉, and A are as defined under formula I above;

preferably A is CH or N, more preferably A is N; R₂ is trifluoromethyl, pentafluoroethyl or trifluoromethylsulfanyl or trifluoromethylsulfonyl; preferably R₂ is trifluoromethyl; and R₉ is hydrogen.

Another preferred group of compounds of formula I is represented by the compounds of formula I-10

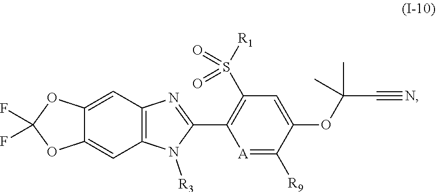

wherein R₁, R₂, R₉, and A are as defined under formula I above; or

an agrochemically acceptable salt, stereoisomer, enantiomer, tautomer or N-oxide of formula I-10.

In one preferred group of compounds of formula I-10, A is CH or N; R₁ is ethyl, propyl or isopropyl or CH₂cyclopropyl; R₃ is C₁-C₂alkyl; R₉ is hydrogen, methyl or ethyl.

In another preferred group of compounds of formula I-10, A is CH or N; R₁ is ethyl; R₃ is methyl; R₉ is hydrogen or methyl, preferably R₉ is hydrogen.

In compounds of formula I-10 and all of the preferred embodiments of compounds of formula I-10 mentioned above, unless otherwise specified, R₁, R₃, R₉, and A are as defined under formula I above;

preferably A is CH or N, more preferably A is N; R₃ is methyl; and R₉ is hydrogen.

Another preferred group of compounds of formula I is represented by the compounds of formula I-11

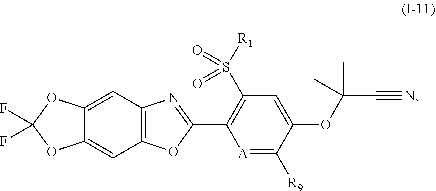

wherein R₁, R₉, and A are as defined under formula I above; or

an agrochemically acceptable salt, stereoisomer, enantiomer, tautomer or N-oxide of formula I-11.

In one preferred group of compounds of formula I-11, A is CH or N; R₁ is ethyl, propyl or isopropyl or CH₂cyclopropyl; and R₉ is hydrogen, methyl or ethyl.

In another preferred group of compounds of formula I-11, A is CH or N; R₁ is ethyl; and R₉ is hydrogen or methyl, preferably R₉ is hydrogen.

In compounds of formula I-11 and all of the preferred embodiments of compounds of formula I-11 mentioned above, unless otherwise specified, R₁, R₉, and A are as defined under formula I above;

preferably A is CH or N, more preferably A is N; and R₉ is hydrogen.

Another especially preferred group of compounds of formula I are those represented by the compounds of formula I-1, I-2, I-3, I-4, I-5, I-6, I-7, I-8, I-9, I-10 or I-11 wherein A is CH or N, preferably A is N;

R₁ is ethyl, propyl, isopropyl or CH₂cyclopropyl; preferably R₁ is ethyl;

R₉ is hydrogen; and

in the case of the compounds of formula I-1, I-2, I-3, I-4, I-5, I-6, I-7, I-8, and I-9 R₂ is trifluoromethyl, pentafluoroethyl, trifluoromethylsulfanyl or trifluoromethylsulfonyl; preferably R₂ is trifluoromethyl; and

in the case of the compounds of formula I-1, I-2, I-3, I-4 and I-10 R₃ is methyl;

and in the case of the compounds of formula I-4 R₄ is ethyl, methoxy or cyclopropyl.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile, improved physico-chemical properties, or increased biodegradability or environmental profile). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

In another aspect the present invention provides a composition comprising an insecticidally, acaricidally, nematicidally or molluscicidally effective amount of a compound of formula (I), or an agrochemically acceptable salt, stereoisomer, enantiomer, tautomer or N-oxide thereof, as defined in any of embodiments 1-25 (above) or any of the embodiments under compounds of formulae I-1, I-2, I-3, I-4, I-5, I-6, I-7, I-8, I-9, I-10 or I-11 and, optionally, an auxiliary or diluent.

In a further aspect the present invention provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in any of embodiments 1-25 (above) or any of the embodiments under compounds of formula I-1, I-2, I-3, I-4, I-5, I-6, I-7, I-8, I-9, I-10 or I-11 (above) or a composition as defined above.

In a yet further aspect, the present invention provides a method for the protection of plant propagation material from the attack by insects, acarines, nematodes or molluscs, which comprises treating the propagation material or the site, where the propagation material is planted, with a composition as defined above.

The process according to the invention for preparing compounds of formula I is carried out by methods known to those skilled in the art. Compounds of formula I, wherein A, R₁, R₉ and Q are defined as under formula I above, may be prepared (scheme 1) by oxidation of compounds of formula II-a, wherein A, R₁, R₉ and Q are defined as under formula I above. The reaction can be performed with reagents such as a peracid, for example peracetic acid or m-chloroperbenzoic acid (mCPBA), or a hydroperoxide, for example hydrogen peroxide or tert-butylhydroperoxide, or an inorganic oxidant, for example a monoperoxo-disulfate salt (oxone), sodium periodate, sodium hypochlorite or potassium permanganate. In a similar way, compounds of formula II-a, wherein A, R₁, R₉ and Q are defined as under formula I above, may be prepared by oxidation of compounds of formula II, wherein A, R₁, R₉ and Q are defined as under formula I above, under analogous conditions described above.

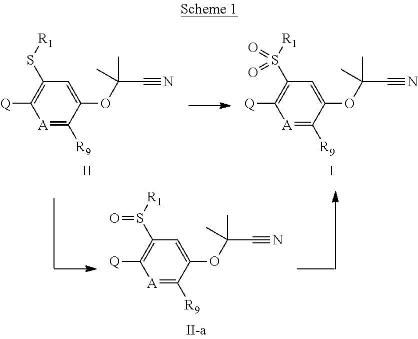

Similarly, compounds of formula II can be oxidized directly into compounds of formula I under the conditions described above. The amount of the oxidant to be used in the reaction is generally 1 to 3 moles, preferably 1 to 1.2 moles, relative to 1 mole of the sulfoxide compounds II-a to produce the sulfone compounds I, and preferably 2 to 2.2 moles of oxidant, relative to 1 mole of the sulfide compounds II to produce the sulfone compounds I. These reactions can be performed in various organic or aqueous solvents compatible to these conditions, at temperatures from below 0° C. up to the boiling point of the solvent system. Examples of the solvent to be used in the reaction include aliphatic halogenated hydrocarbons such as dichloromethane and chloroform; alcohols such as methanol and ethanol; acetic acid; water; and mixtures thereof.

Compounds of formula II, wherein R₉, R₁, A and Q are as defined in formula I,

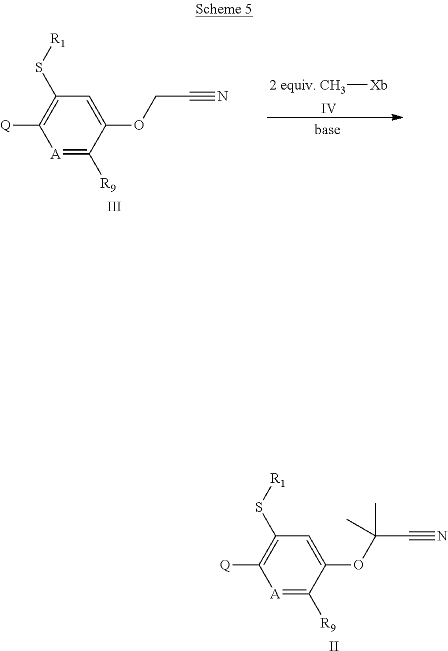

can be prepared (scheme 5) by reacting compounds of formula III, wherein R₉, R₁, A and Q are as defined in formula I, with compounds of formula IV, preferably at least 2 equivalents, wherein Xb is a leaving group such as, for example, chlorine, bromine or iodine (preferably iodine or bromine), or an aryl-, alkyl- or haloalkylsulfonate such as trifluoromethanesulfonate, in presence of a base, preferably at least 2 equivalents, such as, for example, potassium carbonate, cesium carbonate, lithium hexamethyldisilazane or lithium diisopropylamide, in a suitable solvent such as acetonitrile, tetrahydrofuran or N,N-dimethylformamide, at temperatures between −78° C. and 100° C., preferably between −10° C. and 80° C., as described, for example, in Bioorganic & Medicinal Chemistry, 20(18), 5600-5615; 2012. Methyl iodide, methyl bromide or dimethylsulfate are typical representatives of the methylating reagent CH₃—Xb IV. Optionally, compounds of formula III are treated sequentially twice with around each one equivalent (or more) of the methylating reagent CH₃—Xb IV and the base.

The compounds of formula II

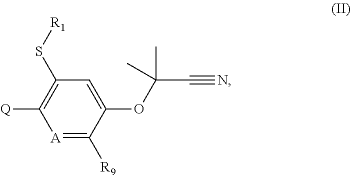

wherein

A, R₁, R₉ and Q are as defined under formula I above, are novel, especially developed for the preparation of the compounds of formula I according to the invention and therefore represent a further object of the invention. The preferences and preferred embodiments of the substituents of the compounds of formula I are also valid for the compounds of formula II.

Compounds of formula III, wherein R₉, R₁, A and Q are as defined in formula I,

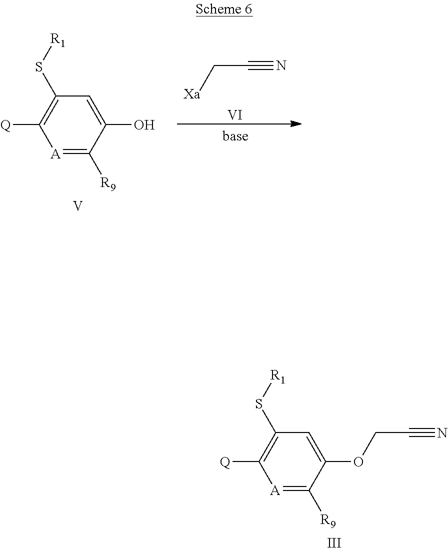

can be prepared (scheme 6) by reacting compounds of formula V, wherein R₉, R₁, A and Q are as defined in formula I, with compounds of formula VI, in which Xa is a leaving group such as, for example, chlorine, bromine or iodine (preferably chlorine or bromine), or an aryl-, alkyl- or haloalkylsulfonate such as trifluoromethanesulfonate, in presence of a base such as, for example, potassium carbonate, cesium carbonate or sodium hydride, optionally in the presence of a catalytic amount of an additive such as sodium or potassium iodide, in a suitable solvent such as acetone, tetrahydrofuran, acetonitrile, dimethylsulfoxide or N,N-dimethylformamide, at temperatures between −10° C. and 100° C., preferably between 0° C. and 80° C., as described, for example, in Tetrahedron Letters, 34(47), 7567-8; 1993.

Alternatively, compounds of formula II, wherein R₉, R₁, A and Q are as defined in formula I,

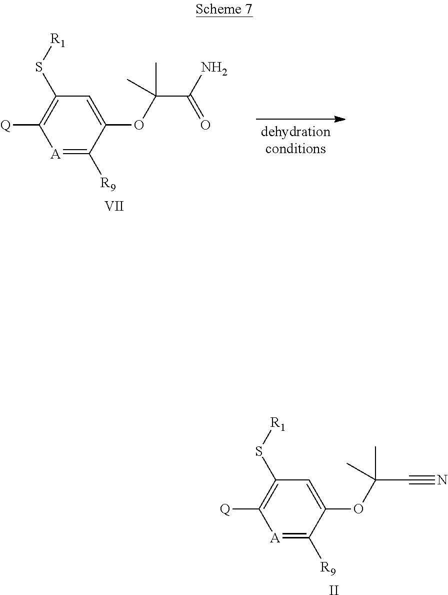

can be prepared (scheme 7) under dehydration conditions by reacting compounds of formula VII, wherein R₉, R₁, A and Q are as defined in formula I, with a dehydrating agent such as trifluoroacetic acid, trifluoroacetic anhydride, phosphorus pentoxide, thionyl chloride or phosphorus oxychloride, optionally in presence of a base such as triethylamine or pyridine, in an appropriate solvent such as for example dichloromethane, dioxane or N,N-dimethylformamide, at temperatures between 0° C. and 180° C., preferably between 5° C. and 80° C., as described, for example, in US 20100267738.

Compounds of formula VII, wherein R₉, R₁, A and Q are as defined in formula I,

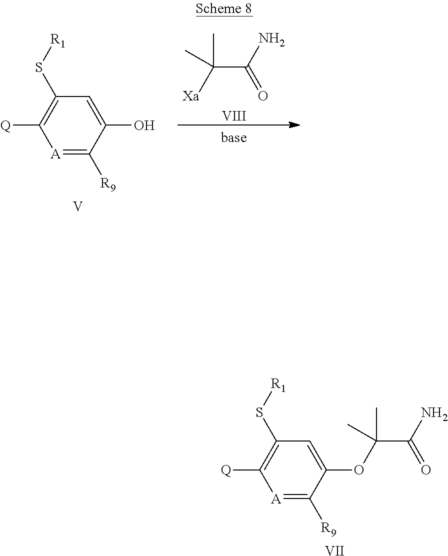

can be prepared (scheme 8) by reacting compounds of formula V, wherein R₉, R₁, A and Q are as defined in formula I, with compounds of formula VIII, wherein Xa is a leaving group such as, for example, chlorine, bromine or iodine (preferably bromine), or an aryl-, alkyl- or haloalkylsulfonate such as trifluoromethanesulfonate, in presence of a base such as, for example, lithium, sodium or potassium hydroxide, sodium hydride, potassium or cesium carbonate, in a suitable solvent such as acetone, dioxane, acetonitrile, N,N-dimethylformamide or N,N-dimethylacetamide, at temperatures between −10° C. and 100° C., preferably between 0° C. and 80° C., as described, for example, in WO 2014071044.

Compounds of formula V, wherein R₉, R₁, A and Q are as defined in formula I,

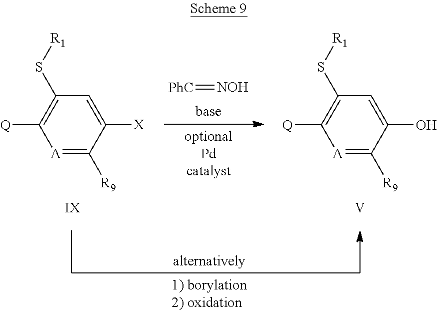

can be prepared (scheme 9) by reacting compounds of formula IX, wherein R₉, R₁, A and Q are as defined in formula I, and in which X is a leaving group such as, for example, chlorine, bromine or iodine (preferably bromine), or an aryl-, alkyl- or haloalkylsulfonate such as trifluoromethanesulfonate, with for example benzaldoxime PhC═NOH, preferably (E)-benzaldehyde oxime, in the presence of a base, such as potassium or cesium carbonate, optionally in the presence of a palladium catalyst such as RockPhos-G3-palladacycle ([(2-Di-tert-butylphosphino-3-methoxy-6-methyl-2′,4′,6′-triisopropyl-1,1′-biphenyl)-2-(2-aminobiphenyl)]palladium(II) methanesulfonate), in an aprotic solvent such as acetonitrile or N,N-dimethylformamide DMF, at temperatures between 0 and 100° C., preferably between room temperature and 80° C., as described, for example, in Angew. Chem. Int. Ed. 56 (16), 4478-4482, 2017.

Alternatively, compounds of formula V, wherein R₉, R₁, A and Q are as defined in formula I, may be prepared from compounds of formula IX, wherein R₉, R₁, A and Q are as defined in formula I, and in which X is a leaving group such as, for example, chlorine, bromine or iodine (preferably bromine), or an aryl-, alkyl- or haloalkylsulfonate such as trifluoromethanesulfonate, by running sequentially 1) a borylation reaction, whereby typically the compound of formula IX is reacted with bispinacol diborane (Bpin)2 under palladium catalysis. Such an introduction of a pinacolborate functional group can be performed in an aprotic solvent, such as dioxane, in presence of a base, preferably a weak base, such as potassium acetate KOAc. [1,1′-Bis(diphenylphosphino)ferrocene]dichloropalladium(II), also known as palladium dppf dichloride or Pd(dppf)Cl2, is a common catalyst for this type of reaction.

Other palladium source/ligand combination involve, for example, tris(dibenzylideneacetone) dipalladium and tricyclohexylphosphine. The temperature of the reaction is preferably performed between 0° C. and the boiling point of the reaction mixture, or the reaction may be performed under microwave irradiation. The intermediate product of this borylation reaction is then further subjected to 2) an oxidation step, whereby typically said intermediate product is treated with hydrogen peroxide H₂O₂, for example a 30% H₂O₂ solution in water, in an inert solvent such as tetrahydrofuran or dioxane, at temperatures between 0 and 100° C., preferably around room temperature. The described process to prepare compounds of the formula V from compounds of the formula IX may include isolation and purification of the borylated intermediate, however this process is also advantageously conducted by engaging said crude intermediate into the oxidation step 2.

Compounds of formula IX, wherein R₉, R₁, A and Q are as defined in formula I, and in which X is a leaving group, in particular those compounds wherein X is a halogen, are known compounds, or can be prepared by known methods, or can be synthesized in analogy to described methods found in the literature. See in particular WO 2016/071214 (Q is Q₂, G₂ is N) and WO 2015/000715 (Q is Q₂, G₂ is CH), WO 2016/026848 and WO 2016/005263 (Q is Q₁, G₁ is CH, G₂ is N), WO 2016/059145 (Q is Q₁, G₁ is N, G₂ is N), WO 2016/020286 and WO 2017/134066 (Q is Q₄), WO 2017/089190, WO 2017/084879 and WO 2016/023954 (Q is Q₅), WO 2015/000715 (Q is Q₃), and WO 2012/086848, WO 2013/018928 (Q is Q₁, G₁ is N or CH, G₂ is CH).

Compounds of formula IV, wherein Xb is a leaving group such as, for example, chlorine, bromine or iodine (preferably iodine or bromine), or an aryl-, alkyl- or haloalkylsulfonate such as trifluoromethanesulfonate; and

compounds of formula VI, wherein Xa is a leaving group such as, for example, chlorine, bromine or iodine (preferably bromine), or an aryl-, alkyl- or haloalkylsulfonate such as trifluoromethanesulfonate; and

compounds of formula VIII, wherein Xa is a leaving group such as, for example, chlorine, bromine or iodine (preferably bromine), or an aryl-, alkyl- or haloalkylsulfonate such as trifluoromethanesulfonate;

are all either known compounds, commercially available or may be prepared by known methods described in the literature.

The subgroup of compounds of the formula V wherein R₉ is C₁-C₄alkyl, defining compounds of the formula Vc, wherein R₁, A and Q are as defined in formula I,

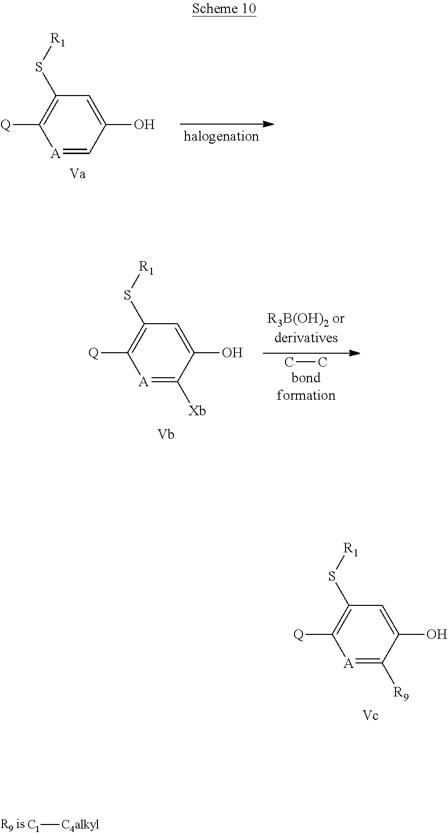

can be prepared (scheme 10) from compounds of formula Vb, wherein R₁, A and Q are as defined in formula I, and in which Xb is halogen, preferably chlorine, bromine or iodine, by means of a C—C bond formation reaction typically under palladium-catalyzed (alternatively nickel-catalyzed) cross-coupling conditions. Such Suzuki-Miyaura cross-coupling reactions between compounds of formula Vb and C₁-C₄alkyl boronic acids of the formula R₉B(OH)₂, wherein R₉ is as defined in formula I, or the corresponding C₁-C₄alkyl boronate ester derivatives, or the corresponding 6-membered tri(C₁-C₄alkyl) boroxine derivatives of the formula (R₉BO)₃, wherein R₉ is as defined in formula I, are well known to a person skilled in the art. In the particular situation where R₉ is methyl, compounds of formula Vb can be reacted, for example, with trimethylboroxine (also known as 2,4,6-trimethyl-1,3,5,2,4,6-trioxatriborinane) in the presence of palladium catalyst, such as tetrakis(triphenylphosphine)-palladium(0) or [1,1′-bis(diphenylphosphino)ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such conditions are described, for example, in Tetrahedron Letters (2000), 41(32), 6237-6240.

Compounds of formula Vb, wherein R₁, A and Q are as defined in formula I, and in which Xb is halogen, preferably chlorine, bromine or iodine, can be prepared by a halogenation reaction, which involves for example, reacting the subgroup of compounds of the formula V wherein R₉ is hydrogen, defining compounds of the formula Va, wherein R₁, A and Q are as defined in formula I, with halogenating reagents such as N-chlorosuccinimide (NCS), N-bromosuccinimide (NBS) or N-iodosuccinimide (NIS), or alternatively chlorine, bromine or iodine, optionally in presence of a base such as sodium, potassium or cesium carbonate. Such halogenation reactions are carried out in an inert solvent, such as chloroform, carbon tetrachloride, 1,2-dichloroethane, acetic acid, ethers, N,N-dimethylformamide, acetonitrile or acetonitrile-water mixtures, at temperatures between 20-200° C., preferably room temperature to 100° C.

Alternatively, compounds of the formula II, wherein Q is Q₁, defining compounds of the formula II-Q₁, wherein R₁, R₉, A, X₁, G₁ and R₂ are as defined in formula I,

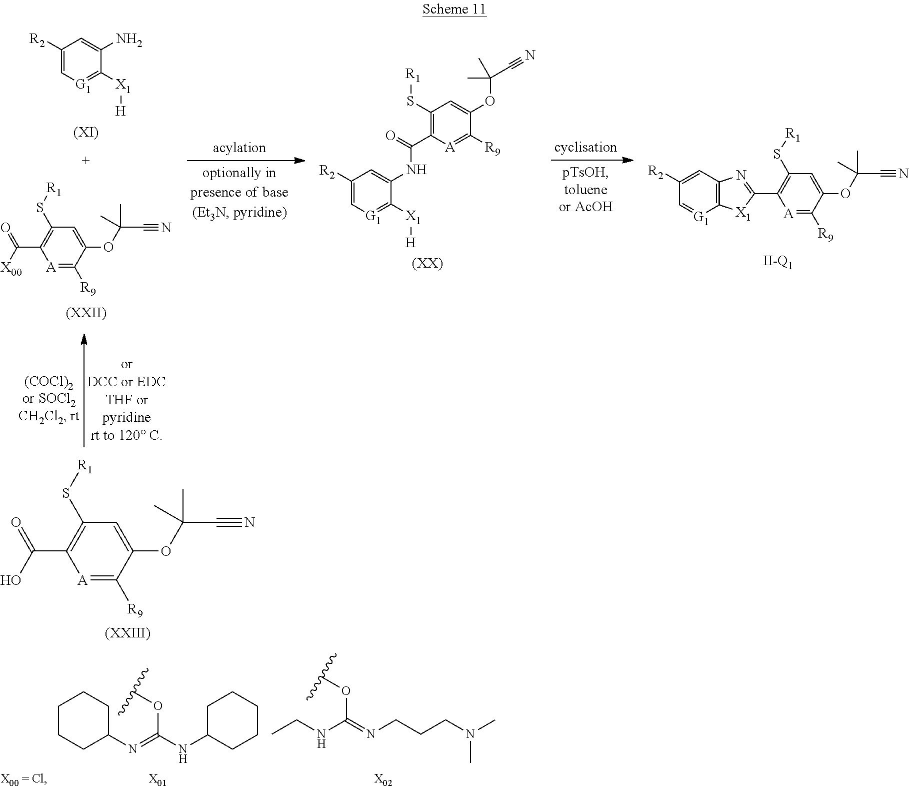

may be prepared (scheme 11) by cyclizing compounds of the formula (XX), wherein R₁, R₉, A, X₁, G₁ and R₂ are as defined in formula I, for example through heating in acetic acid or trifluoroacetic acid (preferably when X₁ is NR₃, wherein R₃ is C₁-C₄alkyl), at temperatures between 0 and 180° C., preferably between 20 and 150° C., optionally under microwave irradiation. Cyclization of compounds of formula (XX) may also be achieved in the presence of an acid catalyst, for example methanesulfonic acid, or para-toluenesulfonic acid p-TsOH, in an inert solvent such as N-methyl pyrrolidone, toluene or xylene, at temperatures between 25-180° C., preferably 100-170° C. Such processes have been described previously, for example, in WO 2010/125985. Alternatively, compounds of formula (XX) may be converted into compounds of formula II-Q1 (preferably when X₁ is O) using triphenylphosphine, diisopropyl azodicarboxylate (or di-ethyl azodicarboxylate) in an inert solvent such as tetrahydrofuran THE at temperatures between 20-50° C. Such Mitsunobu conditions have been previously described for these transformations (see WO 2009/131237).

Compounds of the formula (XX), wherein R₁, R₉, A, X₁, G₁ and R₂ are as defined in formula I, may be prepared via acylation by

i) Activation of compounds of formula (XXIII), wherein R₁, R₉, and A are as defined in formula I, by methods known to those skilled in the art and described in, for example, Tetrahedron, 2005, 61 (46), 10827-10852, to form an activated species (XXII), wherein R₁, R₉, and A are as defined in formula I, and wherein X₀₀ is halogen, preferably chlorine. For example, compounds (XXII) where X₀₀ is halogen, preferably chlorine, are formed by treatment of (XXIII) with, for example, oxalyl chloride (COCl)₂ or thionyl chloride SOCl₂ in the presence of catalytic quantities of N,N-dimethylformamide DMF in inert solvents such as methylene chloride CH₂Cl₂ or tetrahydrofuran THE at temperatures between 20 to 100° C., preferably 25° C. Alternatively, treatment of compounds of formula (XXIII) with, for example, 1-ethyl-3-(3-dimethylaminopropyl)carbodiimide EDC or dicyclohexyl carbodiimide DCC will generate an activated species (XXII), wherein X₀₀ is X₀₁ or X₀₂ respectively, in an inert solvent, such as pyridine or tetrahydrofuran THF, optionally in the presence of a base, such as triethylamine, at temperatures between 50-180° C.; followed by

ii) Treatment of the activated species (XXII) with compounds of the formula (XI), wherein X₁, G₁ and R₂ are as defined in formula I, in the presence of a base, such as triethylamine, N,N-diisopropyl-ethylamine or pyridine, in an inert solvents such as dichloromethane, tetrahydrofuran, dioxane, N,N-dimethylformamide, N,N-dimethylacetamide, acetonitrile, ethyl acetate or toluene, at temperatures between 0 and 50° C., to form the compounds of formula (XX).

Alternatively, compounds of the formula II, wherein Q is Q₅, defining compounds of the formula II-Q₅, wherein R₁, R₉, A, X₁ and R₂ are as defined in formula I,

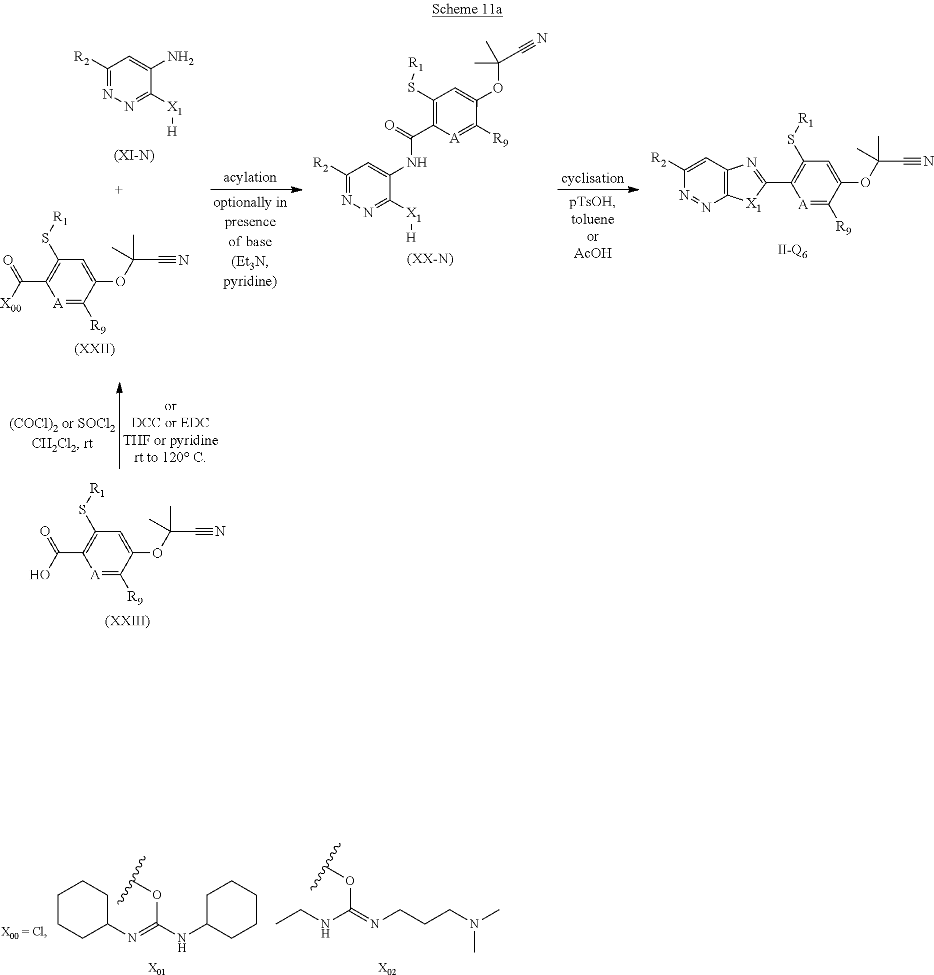

may be prepared (scheme 11a) by cyclizing compounds of the formula (XX-N), wherein R₁, R₉, A, X₁ and R₂ are as defined in formula I, under similar conditions as described above (see text scheme 11). Compounds of the formula (XX-N), wherein R₁, R₉, A, X₁ and R₂ are as defined in formula I, may be prepared by reacting activated species (XXII) described above with compounds of the formula (XI-N), wherein X₁ and R₂ are as defined in formula I, under similar conditions as described above (see text scheme 11).

Compounds of formula (XXIII), wherein R₁, R₉, and A are as defined in formula I,

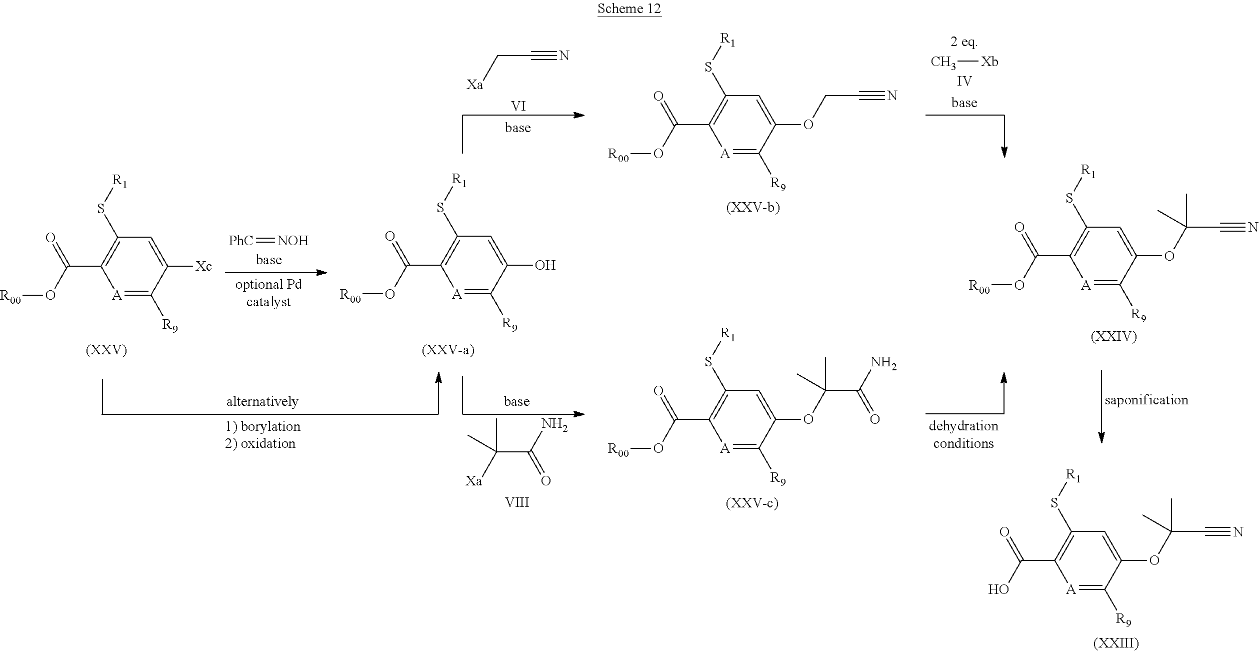

may be prepared (scheme 12) by saponification of compounds of formula (XXIV), wherein R₁, R₉, and A are as defined in formula I, and in which R₀₀ is C₁-C₆alkyl, under conditions known to a person skilled in the art (using for example conditions such as: aqueous sodium, potassium or lithium hydroxide in methanol, ethanol, tetrahydrofuran or dioxane at room temperature, or up to refluxing conditions).

Compounds of formula (XXIV), wherein R₁, R₉, and A are as defined in formula I, and in which R₀₀ is C₁-C₆alkyl, may be prepared by reacting compounds of formula (XXV-b), wherein R₉, R₁ and A are as defined in formula I, and in which R₀₀ is C₁-C₆alkyl, with compounds of formula IV, wherein Xb is a leaving group such as, for example, chlorine, bromine or iodine (preferably iodine or bromine), or an aryl-, alkyl- or haloalkylsulfonate such as trifluoromethanesulfonate, under conditions already described above (see scheme 5, transformation of compounds III into II).

Compounds of formula (XXV-b), wherein R₉, R₁ and A are as defined in formula I, and in which R₀₀ is C₁-C₆alkyl, may be prepared by reacting compounds of formula (XXV-a), wherein R₁, R₉ and A are as defined in formula I, and in which R₀₀ is C₁-C₆alkyl, with compounds of formula VI, in which Xa is a leaving group such as, for example, chlorine, bromine or iodine (preferably chlorine or bromine), or an aryl-, alkyl- or haloalkylsulfonate such as trifluoromethanesulfonate, under conditions already described above (see scheme 6, transformation of compounds V into III).

Alternatively, compounds of formula (XXIV), wherein R₁, R₉, and A are as defined in formula I, and in which R₀₀ is C₁-C₆alkyl, may be prepared by submitting compounds of formula (XXV-c), wherein R₁, R₉, and A are as defined in formula I, and in which R₀₀ is C₁-C₆alkyl, to dehydration conditions already described above (see scheme 7, transformation of compounds VII into II).

Compounds of formula (XXV-c), wherein R₁, R₉, and A are as defined in formula I, and in which R₀₀ is C₁-C₆alkyl, may be prepared by reacting compounds of formula (XXV-a), wherein R₁, R₉ and A are as defined in formula I, and in which R₀₀ is C₁-C₆alkyl, with compounds of formula VIII, wherein Xa is a leaving group such as, for example, chlorine, bromine or iodine (preferably bromine), or an aryl-, alkyl- or haloalkylsulfonate such as trifluoromethanesulfonate, under conditions already described above (see scheme 8, transformation of compounds V into VII).

Compounds of formula (XXV-a), wherein R₁, R₉ and A are as defined in formula I, and in which R₀₀ is C₁-C₆alkyl, may be prepared by reacting compounds of formula (XXV), wherein R₁, R₉ and A are as defined in formula I, and wherein R₀₀ is C₁-C₆alkyl, and in which Xc is a leaving group such as, for example, chlorine, bromine or iodine (preferably bromine), or an aryl-, alkyl- or haloalkyl-sulfonate such as trifluoromethanesulfonate, with for example benzaldoxime PhC═NOH, preferably (E)-benzaldehyde oxime, under conditions already described above (see scheme 9, transformation of compounds IX into V). Alternatively, the process to prepare compounds of the formula (XXV-a) from compounds of the formula (XXV) may also involve the borylation/oxidation conditions also already described in scheme 9.

Compounds of formula (XXV), wherein R₁, R₉ and A are as defined in formula I, and wherein R₀₀ is C₁-C₆alkyl, and in which Xc is a leaving group such as, for example, chlorine, bromine or iodine, or an aryl-, alkyl- or haloalkylsulfonate such as trifluoromethanesulfonate, in particular those compounds wherein Xc is a halogen (even more preferably chlorine, bromine or iodine), are either known compounds, commercially available or may be prepared by known methods, described in the literature, as for example in WO 2016/005263, WO 2016/023954, WO 2016/026848 and WO 2016/104746.

Alternatively, compounds of the formula II, wherein Q is Q₂, defining compounds of the formula II-Q₂, wherein R₁, R₉, A and R₂ are as defined in formula I,

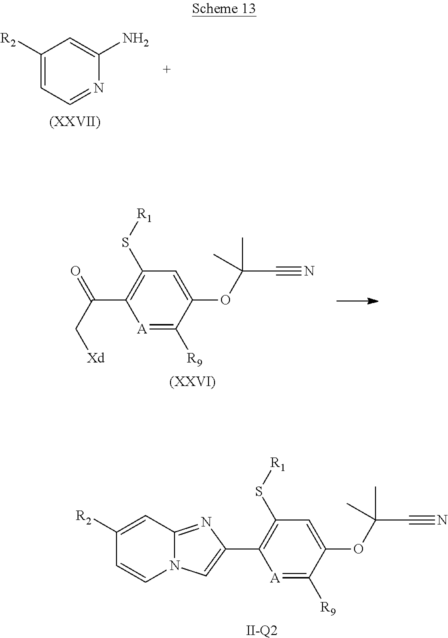

can be prepared (scheme 13) by condensing compounds of the formula (XXVI), wherein R₁, R₉, and A are as defined in formula I, and in which Xd is a leaving group such as, for example, chlorine, bromine or iodine (preferably chlorine or bromine), with compounds of the formula (XXVII), wherein R₂ is as defined in formula I, in an inert solvent, for example ethanol or acetonitrile, optionally in the presence of a suitable base, such as sodium, potassium or cesium carbonate, or magnesium oxide, at temperatures between 50 and 150° C., optionally under microwave heating conditions. Such processes have been described previously, for example, in WO 2012/49280 or WO 2003/031587. Compounds of formula (XXVII), wherein R₂ is as defined in formula I, are either known compounds, commercially available or may be prepared by known methods known to those skilled in the art, see in particular WO 2016/071214.

Compounds of the formula (XXVI), wherein R₁, R₉, and A are as defined in formula I, and in which Xd is a leaving group such as, for example, chlorine, bromine or iodine (preferably chlorine or bromine),

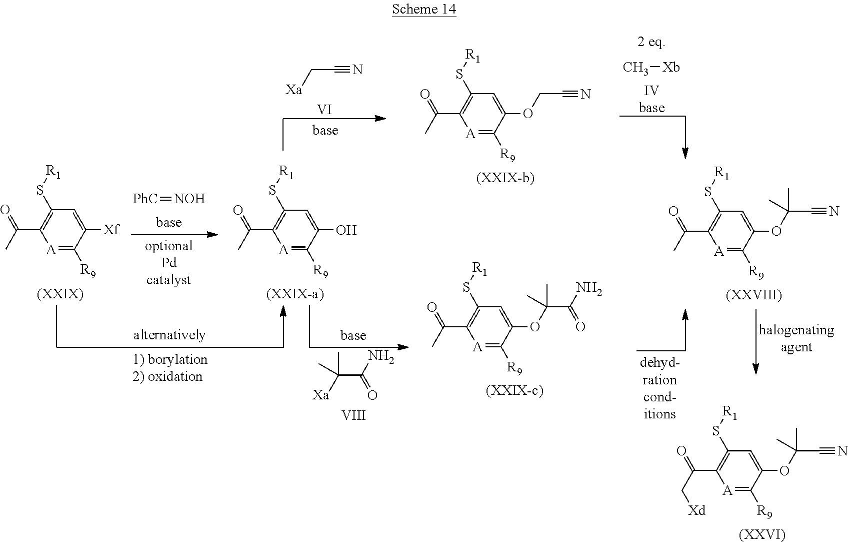

can be prepared (scheme 14) by treatment of compounds of formula (XXVIII), wherein R₁, R₉, and A are as defined in formula I, with a halogenating agent (“Xd*” source), e.g. N-bromosuccinimide, N-iodosuccinimide, N-chlorosuccinimide, I₂, CuBr₂, Br₂ in acetic acid, ortrimethyl(phenyl)ammonium tribromide PhNMe₃*Br₃⁻, typically in a solvent such as methanol, acetonitrile, tetrahydrofuran, ethyl acetate, chloroform or dichloromethane, or mixtures thereof, at temperatures between 0° C. and 150° C., preferably between room temperature and 120° C., optionally under microwave heating conditions. Such processes have been described previously, for example, in WO2016/071214.

Compounds of formula (XXVIII), wherein R₁, R₉, and A are as defined in formula I, may be prepared by reacting compounds of formula (XXIX-b), wherein R₉, R₁ and A are as defined in formula I, with compounds of formula IV, wherein Xb is a leaving group such as, for example, chlorine, bromine or iodine (preferably iodine or bromine), or an aryl-, alkyl- or haloalkylsulfonate such as trifluoromethanesulfonate, under conditions already described above (see scheme 5, transformation of compounds III into II).

Compounds of formula (XXIX-b), wherein R₉, R₁ and A are as defined in formula I, may be prepared by reacting compounds of formula (XXIX-a), wherein R₁, R₉ and A are as defined in formula I, with compounds of formula VI, in which Xa is a leaving group such as, for example, chlorine, bromine or iodine (preferably chlorine or bromine), or an aryl-, alkyl- or haloalkylsulfonate such as trifluoromethanesulfonate, under conditions already described above (see scheme 6, transformation of compounds V into III).

Alternatively, compounds of formula (XXVIII), wherein R₁, R₉, and A are as defined in formula I, can be prepared by submitting compounds of formula (XXIX-c), wherein R₁, R₉, and A are as defined in formula I, to dehydration conditions already described above (see scheme 7, transformation of compounds VII into II).

Compounds of formula (XXIX-c), wherein R₁, R₉, and A are as defined in formula I, can be prepared by reacting compounds of formula (XXIX-a), wherein R₁, R₉ and A are as defined in formula I, with compounds of formula VIII, wherein Xa is a leaving group such as, for example, chlorine, bromine or iodine (preferably bromine), or an aryl-, alkyl- or haloalkylsulfonate such as trifluoromethanesulfonate, under conditions already described above (see scheme 8, transformation of compounds V into VII).

Compounds of formula (XXIX-a), wherein R₁, R₉ and A are as defined in formula I, can be prepared by reacting compounds of formula (XXIX), wherein R₁, R₉ and A are as defined in formula I, and in which Xf is a leaving group such as, for example, chlorine, bromine or iodine (preferably bromine), or an aryl-, alkyl- or haloalkyl-sulfonate such as trifluoromethanesulfonate, with for example benzaldoxime PhC═NOH, preferably (E)-benzaldehyde oxime, under conditions already described above (see scheme 9, transformation of compounds IX into V). Alternatively, the process to prepare compounds of the formula (XXIX-a) from compounds of the formula (XXIX) may also involve the borylation/oxidation conditions also already described in scheme 9.

Compounds of formula (XXIX), wherein R₁, R₉ and A are as defined in formula I, and wherein Xf is a leaving group such as, for example, chlorine, bromine or iodine, or an aryl-, alkyl- or haloalkylsulfonate such as trifluoromethanesulfonate, in particular those compounds wherein Xf is a halogen (even more preferably chlorine, bromine or iodine; particularly preferred is chlorine or bromine), are either known compounds, commercially available or may be prepared by known methods, described in the literature, as for example in WO 2016/071214.

Alternatively, compounds of the formula II, wherein Q is Q₅, defining compounds of the formula II-Q₅, wherein R₁, R₉, A, R₃, R₄ and R₂ are as defined in formula I,

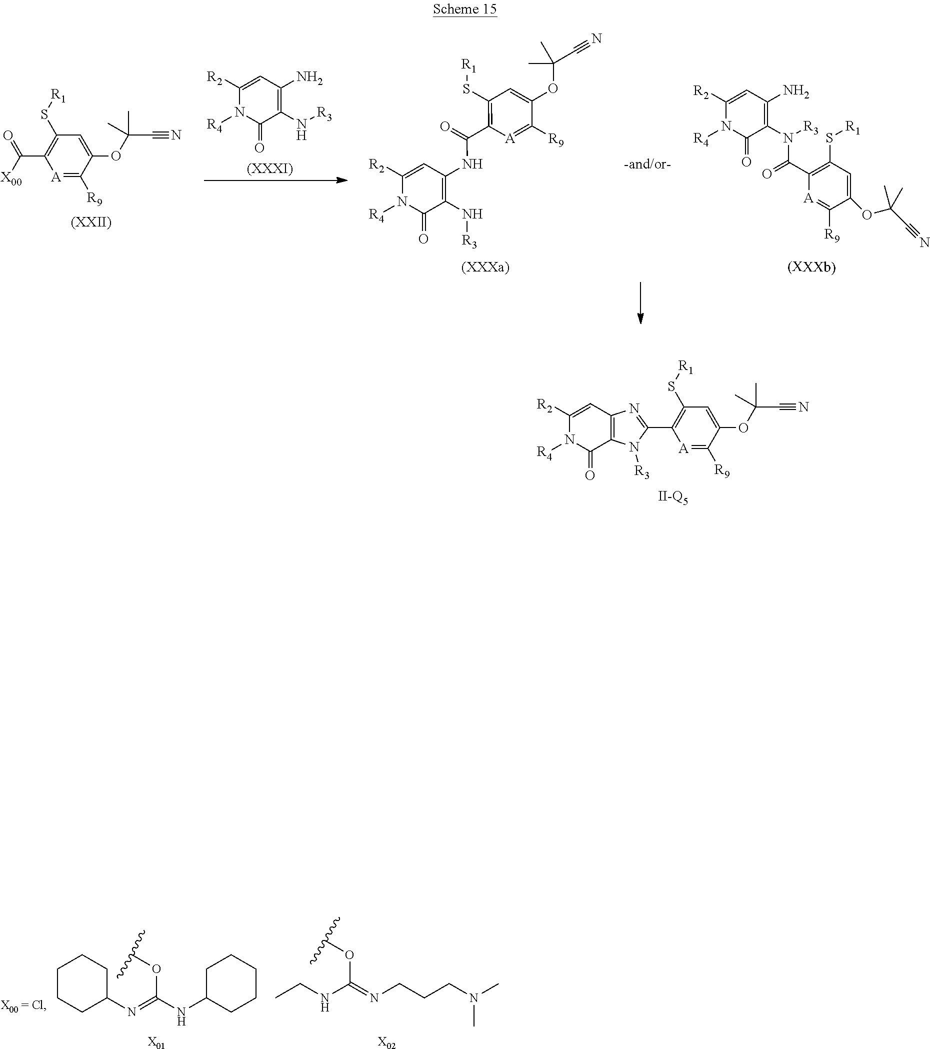

may be prepared (scheme 15) by cyclizing compounds of the formula (XXXa), wherein R₁, R₉, A, R₃, R₄ and R₂ are as defined in formula I, or regioisomers of the formula (XXXb) with identical substituent definitions, or a mixture thereof in any ratio, under conditions already described above (see scheme 11, transformation of compounds (XX) into II-Q₁).

Compounds of the formula (XXXa), wherein R₁, R₉, A, R₃, R₄ and R₂ are as defined in formula I, or regioisomers of the formula (XXXb) with identical substituent definitions, or a mixture thereof in any ratio, may be prepared by treatment of the activated species (XXII) described above with compounds of the formula (XXXI), wherein R₃, R₄ and R₂ are as defined in formula I, under conditions already described above (see scheme 11, transformation of compounds (XXII) and (XI) into compounds (XX)).

Compounds of formula (XXXI), wherein R₃, R₄ and R₂ are as defined in formula I, have been previously described, for example, in WO 2016/023954, WO 2016/142326, and WO 2017/133994.

Alternatively, compounds of the formula II, wherein Q is Q₃, defining compounds of the formula II-Q₃, wherein R₁, R₉, A and R₂ are as defined in formula I,

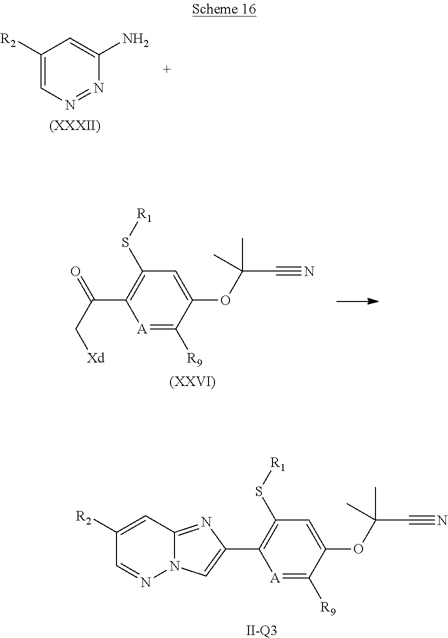

may be prepared (scheme 16) by condensing compounds of the formula (XXVI) described above, wherein R₁, R₉, and A are as defined in formula I, and in which Xd is a leaving group such as, for example, chlorine, bromine or iodine (preferably chlorine or bromine), with compounds of the formula (XXXII), wherein R₂ is as defined in formula I, in an inert solvent, for example ethanol, toluene or acetonitrile, optionally in the presence of a suitable base, such as sodium, potassium or cesium carbonate (or sodium or potassium hydrogen carbonate) at temperatures between 50 and 150° C., optionally under microwave heating conditions. Such processes have been described previously, for example, in WO 2011/074658. Compounds of formula (XXXII), wherein R₂ is as defined in formula I, are either known compounds, commercially available or may be prepared by known methods known to those skilled in the art (see for example WO 2011/074658 and WO 2010/083145).

Alternatively, compounds of the formula II, wherein Q is Q₄, defining compounds of the formula II-Q₄, wherein R₁, R₉, A, G₁, G₂ and R₂ are as defined in formula I,

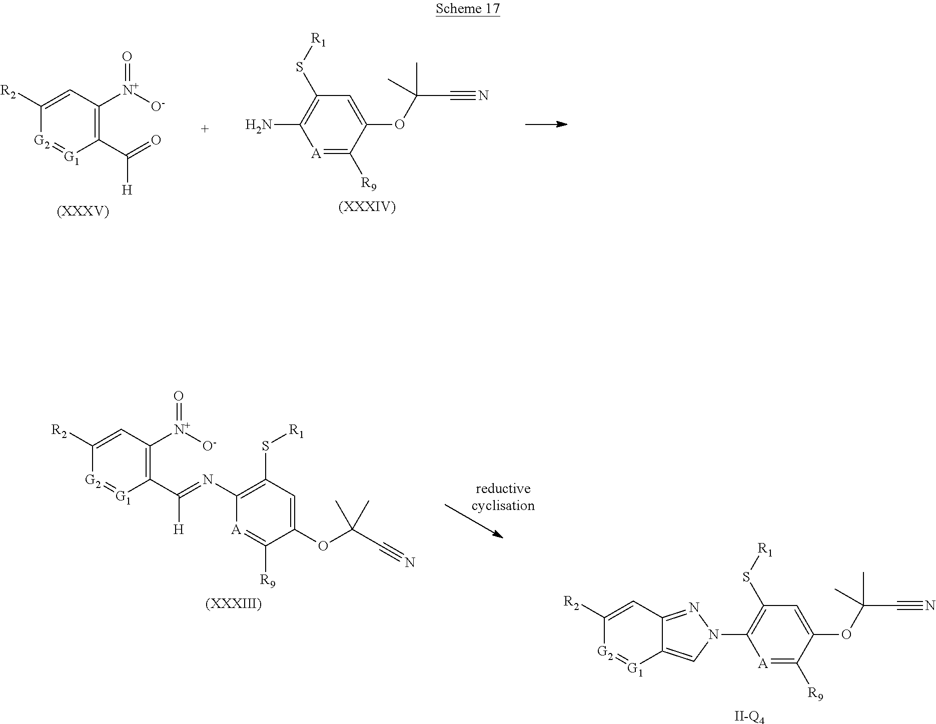

may be prepared (scheme 17) by reductive cyclisation of compounds of the formula (XXXIII), wherein R₁, R₉, A, G₁, G₂ and R₂ are as defined in formula I, in the presence of a reducing agent such as trialkyl phosphite (more specifically, for example, triethyl phosphite), trialkylphosphine or triphenylphosphine. The principle of this reductive cyclisation is analogous to the known Cadogan reaction. Alternatively, this reaction may be conducted in presence of a metal catalyst, for example a molybdenum(VI) catalyst, such as MoO₂Cl₂(dmf)₂ [molybdenyl chloride-bis(dimethylformamide)], or more generally with transition metal complexes, in combination with a reducing agent such as triethylphosphite, triphenylphosphine or CO. Suitable solvents may include use of excess of the reducing agent (such as triethyl phosphite), or for example toluene or xylene, at temperatures between room temperature and 200° C., preferably between 50 and 160° C., optionally under microwave heating conditions. Such reductive cyclisation reaction conditions were described in, for example, WO 2017/134066.

Compounds of the formula (XXXIII), wherein R₁, R₉, A, G₁, G₂ and R₂ are as defined in formula I, may be prepared by reaction between compounds of formula (XXXIV), wherein R₁, R₉, and A are as defined in formula I, and compounds of formula (XXXV), wherein G₁, G₂ and R₂ are as defined in formula I, usually upon heating at temperatures between room temperature and 200° C., preferably between 40 and 160° C., optionally under microwave heating conditions, in suitable solvents that may include, for example, toluene or xylene. The formation of compounds of formula (XXXIII) may require water removal, either by azeotropical distillation, or by means of a drying agent such as for example TiCl₄ or molecular sieves. Such formation of Schiff bases of formula (XXXIII) is known to those skilled in the art, and was described in, for example, WO 2017/134066.

Compounds of formula (XXXV), wherein G₁, G₂ and R₂ are as defined in formula I, are either known compounds, commercially available or may be prepared by known methods known to those skilled in the art.

Compounds of formula (XXXIV), wherein R₁, R₉, and A are as defined in formula I,

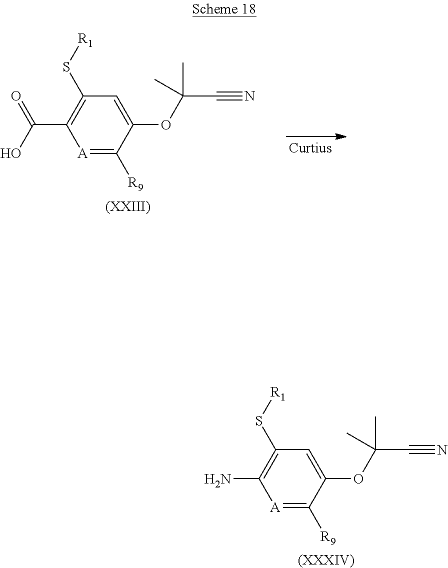

may be prepared (scheme 18) by submitting compounds of formula (XXIII), described above (or their corresponding activated species (XXII) also described above) to Curtius rearrangement/degradation conditions known to those skilled in the art. Such conditions have been described, for example, in WO 2009099086 and Journal of Medicinal Chemistry, 55(22), 9589-9606; 2012.

Alternatively, compounds of the formula II, wherein Q is Q₇ and X₁ is NR₃, defining compounds of the formula II-Q₇-a, wherein R₁, R₉, A and R₃ are as defined in formula I,

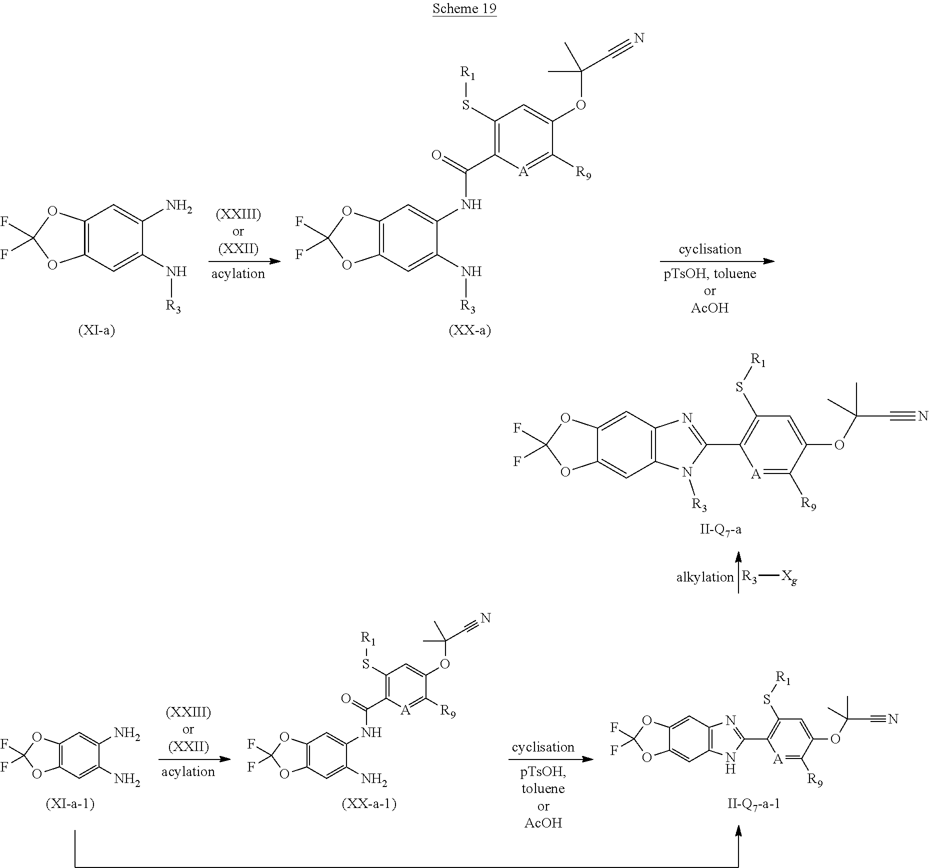

can be prepared (scheme 19) by cyclizing compounds of the formula (XX-a), wherein R₁, R₉, A and R₃ are as defined in formula I, under conditions already described above (see scheme 11, transformation of compounds (XX) into II-Q₁).

Compounds of the formula (XX-a), wherein R₁, R₉, A and R₃ are as defined in formula I, may be prepared by reacting activated species (XXII) described above with compounds of the formula (XI-a), or a salt thereof, wherein R₃ is as defined in formula I, under similar acylation conditions as described above (see text scheme 11).

Alternatively, compounds of the formula (XX-a), wherein R₁, R₉, A and R₃ are as defined in formula I, can also be prepared by reacting compounds of formula (XXIII) described above with compounds of the formula (XI-a), or a salt thereof, wherein R₃ is as defined in formula I, in the presence of an activating agent, such as propanephosphonic acid anhydride (T3P), carbodiimides (such as dicyclohexylcarbodiimide (DCC), diisopropylcarbodiimide (DIC) and 1-ethyl-3-(3-dimethylamino-propyl)carbodiimide (EDC)), optionally in the presence of a suitable base, such as triethylamine, diisopropylethylamine or pyridine, optionally in the presence of an acylation catalyst, such as 4-dimethylamino-pyridine (DMAP), in an appropriate solvent such as dichloromethane, tetrahydrofuran, 2-methyltetrahydrofuran, dioxane, N,N-dimethylformamide, N,N-dimethylacetamide, N-methyl-pyrrolidone, acetonitrile, ethyl acetate, toluene, xylene or chlorobenzene and any mixtures thereof, and at temperatures between 0° C. to approximately 80° C.

Alternatively, compounds of the formula II-Q₇-a, wherein R₁, R₉, A and R₃ are as defined in formula I, may be prepared by an alkylation reaction of compounds of the formula II-Q₇-a-1, wherein R₁, R₉ and A are as defined in formula I, with reagents of the formula R₃—X_{g}, wherein R₃ is as defined in formula I and X_{g} is a leaving group such as, for example, chlorine, bromine or iodine (preferably iodine or bromine), or an aryl-, alkyl- or haloalkylsulfonate such as trifluoromethanesulfonate, in the presence of a base such as, for example, potassium carbonate, cesium carbonate, lithium hexamethyldisilazane or lithium diisopropylamide, in a suitable solvent such as acetonitrile, tetrahydrofuran or N,N-dimethylformamide, at temperatures between −78° C. and 100° C., preferably between −10° C. and 80° C. In the particular situation wherein R₃ is CH₃, methyl iodide, methyl bromide or dimethylsulfate are typical representatives of the alkylating reagent R₃-X_{g}.

Compounds of the formula II-Q₇-a-1, wherein R₁, R₉ and Aare as defined in formula I, may be prepared by cyclization of compounds of the formula (XX-a-1), wherein R₁, R₉ and A are as defined in formula I, under conditions already described above (see scheme 19, transformation of compounds (XX-a) into II-Q₇-a).

Compounds of the formula (XX-a-1), wherein R₁, R₉ and Aare as defined in formula I, may be prepared by reacting activated species (XXII) described above with compounds of the formula (XI-a-1), or a salt thereof, under similar acylation conditions as described above (see scheme 19, transformation of compounds (XI-a) into (XX-a)). Or alternatively, compounds of the formula (XX-a-1), wherein R₁, R₉ and A are as defined in formula I, may also be prepared by reacting compounds of formula (XXIII) described above with compounds of the formula (XI-a-1), or a salt thereof, under similar acylation conditions as described above (see scheme 19, transformation of compounds (XI-a) into (XX-a)).

As a further alternative, compounds of the formula II-Q₇-a-1, wherein R₁, R₉ and Aare as defined in formula I, may be prepared by the direct condensation of compounds of the formula (XI-a-1), or a salt thereof, with compounds of formula (XXII) or (XXIII) described above, under analogous conditions described, for example, in WO20/013147.

Alternatively, compounds of the formula II, wherein Q is Q₇ and X₁ is O, defining compounds of the formula II-Q₇-b, wherein R₁, R₉, and A are as defined in formula I,

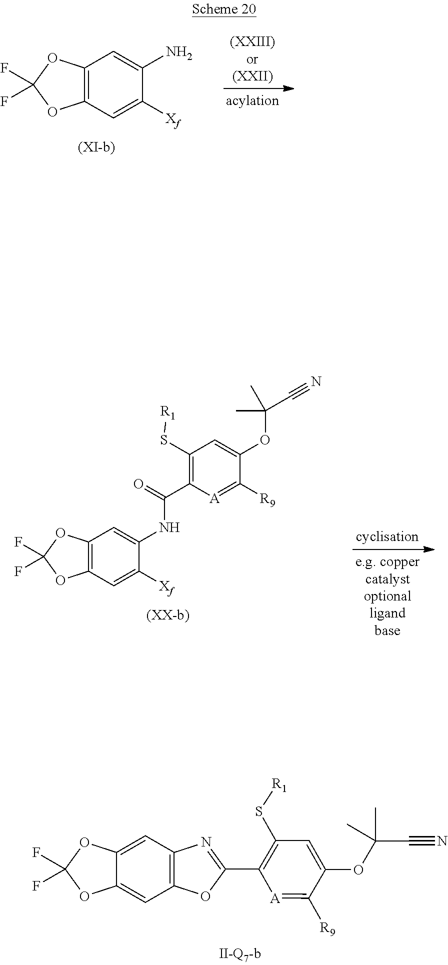

can be prepared (scheme 20) by cyclizing compounds of the formula (XX-b), wherein R₁, R₉, A and R₃ are as defined in formula I, and in which X_{f} is a halogen leaving group, such as, for example, chlorine, bromine or iodine (preferably chlorine or bromine), in the presence of a base, such as sodium carbonate, potassium carbonate or cesium carbonate, or potassium tert-butoxide, in the presence of a metal catalyst, for example a copper catalyst such as copper(I) iodide, optionally in the presence of a ligand, for example a diamine ligands (e.g. N,N′-dimethylethylenediamine or trans-cyclohexyldiamine) or dibenzylideneacetone (dba), or 1,10-phenanthroline, in a solvent such as toluene, N,N-dimethylformamide DMF, N-methyl pyrrolidone NMP, dimethyl sulfoxide DMSO, dioxane, or tetrahydrofuran THF, at temperatures between 30-180° C., optionally under microwave irradiation. Compounds of the formula (XX-b), wherein R₁, R₉, A and R₃ are as defined in formula I, and in which X_{f} is a halogen leaving group, such as, for example, chlorine, bromine or iodine (preferably chlorine or bromine), may be prepared by reacting activated species (XXII) described above with compounds of the formula (XI-b), or a salt thereof, wherein Xf is a halogen leaving group, such as, for example, chlorine, bromine or iodine (preferably chlorine or bromine), under similar acylation conditions as described above (see text scheme 11).

Alternatively, compounds of the formula (XX-b), wherein R₁, R₉, A and R₃ are as defined in formula I, can also be prepared by reacting compounds of formula (XXIII) described above with compounds of the formula (XI-b), or a salt thereof, wherein Xf is a halogen leaving group, such as, for example, chlorine, bromine or iodine (preferably chlorine or bromine), in the presence of an activating agent, such as propanephosphonic acid anhydride (T3P), carbodiimides (such as dicyclohexylcarbodiimide (DCC), diisopropylcarbodiimide (DIC) and 1-ethyl-3-(3-dimethylamino-propyl)carbodiimide (EDC)), optionally in the presence of a suitable base, such as triethylamine, diisopropylethylamine or pyridine, optionally in the presence of an acylation catalyst, such as 4-dimethylamino-pyridine (DMAP), in an appropriate solvent such as dichloromethane, tetrahydrofuran, 2-methyltetrahydrofuran, dioxane, N,N-dimethylformamide, N,N-dimethylacetamide, N-methyl-pyrrolidone, acetonitrile, ethyl acetate, toluene, xylene or chlorobenzene and any mixtures thereof, and at temperatures between 0° C. to approximately 80° C.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The compounds of formula XXXVI

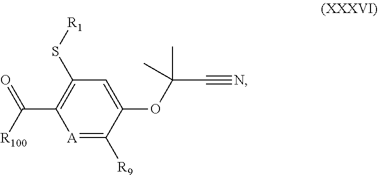

wherein

R₁, R₉ and A are as defined under formula I above, and R₁₀₀ is OH, chloro or C₁-C₄alkoxy, are novel, especially developed for the preparation of the compounds of formula I according to the invention and therefore represent a further object of the invention. The preferences and preferred embodiments of the substituents of the compounds of formula I are also valid for the compounds of formula XXXVI.

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 00/15615.

Compounds wherein R₂ is C₁-C₄haloalkylsulfinyl or C₁-C₄haloalkylsulfonyl may be prepared from the corresponding compounds wherein R₂ is C₁-C₄haloalkylsulfanyl with suitable oxidation methods described, for example, in WO 19/008115.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula I according to the following Tables X, A-1 to A-22 and B-1 to B-4 below can be prepared according to the methods described above. The examples which follow are intended to illustrate the invention and show preferred compounds of formula I.

The Tables A-1 to A-22 below illustrate specific compounds of the invention.

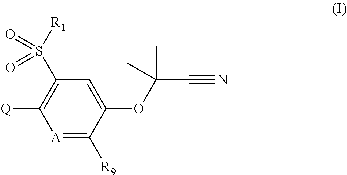

Table A-1 provides 4 compounds A-1.001 to A-1.004 of formula I wherein R₁ is ethyl, and A and R₉ are as defined in Table X, and Q is taken from the group of formula Q₆ as

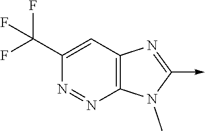

TABLE X
Examples of exotic woodborers of economic importance.

| Index | A | R₉ |
|---|---|---|
| 1 | N | H |
| 2 | N | CH₃ |
| 3 | CH | H |
| 4 | CH | CH₃ |

For example, compound A-1.004 has the following structure:

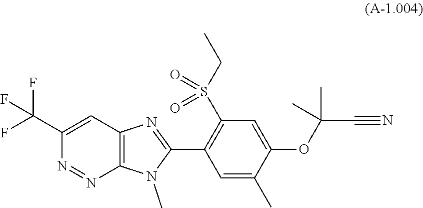 [SMILES: CCS(=O)(=O)c1cc(OC(C)(C)C#N)c(C)cc1-c1nc2cc(C(F)(F)F)nnc2n1C]

Table A-2 provides 4 compounds A-2.001 to A-2.004 of formula I wherein R₁ is ethyl, and A and R₉ are as defined in Table X, and Q is taken from the group of formula Q₆ as

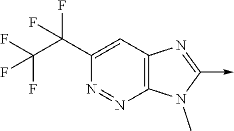

Table A-3 provides 4 compounds A-3.001 to A-3.004 of formula I wherein R₁ is ethyl, and A and R₉ are as defined in Table X, and Q is taken from the group of formula Q₁ as

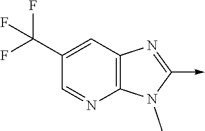

Table A-4 provides 4 compounds A-4.001 to A-4.004 of formula I wherein R₁ is ethyl, and A and R₉ are as defined in Table X, and Q is taken from the group of formula Q₁ as

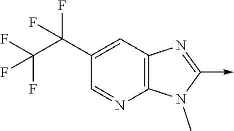

Table A-5 provides 4 compounds A-5.001 to A-5.004 of formula I wherein R₁ is ethyl, and A and R₉ are as defined in Table X, and Q is taken from the group of formula Q₂ as

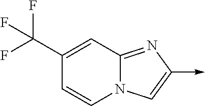

Table A-6 provides 4 compounds A-6.001 to A-6.004 of formula I wherein R₁ is ethyl, and A and R₉ are as defined in Table X, and Q is taken from the group of formula Q₃ as

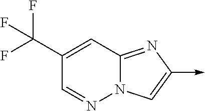

Table A-7 provides 4 compounds A-7.001 to A-7.004 of formula I wherein R₁ is ethyl, and A and R₉ are as defined in Table X, and Q is taken from the group of formula Q₁ as

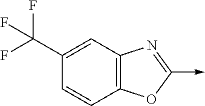

Table A-8 provides 4 compounds A-8.001 to A-8.004 of formula I wherein R₁ is ethyl, and A and R₉ are as defined in Table X, and Q is taken from the group of formula Q₁ as

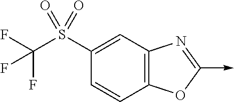

Table A-9 provides 4 compounds A-9.001 to A-9.004 of formula I wherein R₁ is ethyl, and A and R₉ are as defined in Table X, and Q is taken from the group of formula Q₄ as

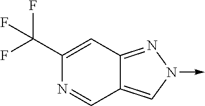

Table A-10 provides 4 compounds A-10.001 to A-10.004 of formula I wherein R₁ is ethyl, and A and R₉ are as defined in Table X, and Q is taken from the group of formula Q₄ as

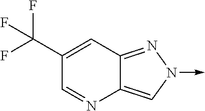

Table A-11 provides 4 compounds A-11.001 to A-11.004 of formula I wherein R₁ is ethyl, and A and R₉ are as defined in Table X, and Q is taken from the group of formula Q₅ as

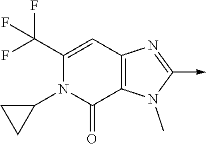

Table A-12 provides 4 compounds A-12.001 to A-12.004 of formula I wherein R₁ is ethyl, and A and R₉ are as defined in Table X, and Q is taken from the group of formula Q₅ as

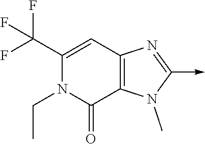

Table A-13 provides 4 compounds A-13.001 to A-13.004 of formula I wherein R₁ is ethyl, and A and R₉ are as defined in Table X, and Q is taken from the group of formula Q₅ as

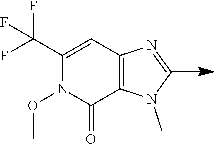 [SMILES: COn1c(C(F)(F)F)cc2ncn(C)c2c1=O]

Table A-14 provides 4 compounds A-14.001 to A-14.004 of formula I wherein R₁ is ethyl, and A and R₉ are as defined in Table X, and Q is taken from the group of formula Q₅ as

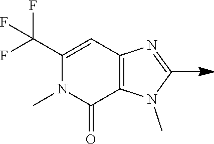 [SMILES: Cn1c(C(F)(F)F)cc2ncn(C)c2c1=O]

Table A-15 provides 4 compounds A-15.001 to A-15.004 of formula I wherein R₁ is ethyl, and A and R₉ are as defined in Table X, and Q is taken from the group of formula Q₁ as

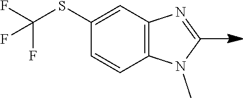

Table A-16 provides 4 compounds A-16.001 to A-16.004 of formula I wherein R₁ is ethyl, and A and R₉ are as defined in Table X, and Q is taken from the group of formula Q₁ as

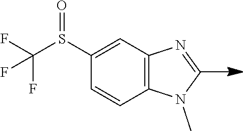 [SMILES: Cn1cnc2cc(S(=O)C(F)(F)F)ccc21]

Table A-17 provides 4 compounds A-17.001 to A-17.004 of formula I wherein R₁ is ethyl, and A and R₉ are as defined in Table X, and Q is taken from the group of formula Q₁ as

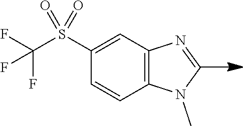 [SMILES: Cn1cnc2cc(S(=O)(=O)C(F)(F)F)ccc21]

Table A-18 provides 4 compounds A-18.001 to A-18.004 of formula I wherein R₁ is ethyl, and A and R₉ are as defined in Table X, and Q is taken from the group of formula Q₁ as

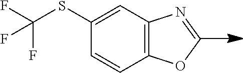 [SMILES: FC(F)(F)Sc1ccc2ocnc2c1]

Table A-19 provides 4 compounds A-19.001 to A-19.004 of formula I wherein R₁ is ethyl, and A and R₉ are as defined in Table X, and Q is taken from the group of formula Q₁ as

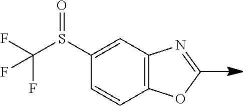 [SMILES: O=S(c1ccc2ocnc2c1)C(F)(F)F]

Table A-20 provides 4 compounds A-20.001 to A-20.004 of formula I wherein R₁ is ethyl, and A and R₉ are as defined in Table X, and Q is taken from the group of formula Q₂ as

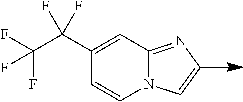 [SMILES: FC(F)(F)C(F)(F)c1ccn2ccnc2c1]

Table A-21 provides 4 compounds A-21.001 to A-21.004 of formula I wherein R₁ is ethyl, and A and R₉ are as defined in Table X, and Q is taken from the group of formula Q₇ as

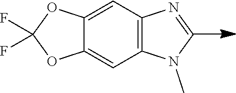

Table A-22 provides 4 compounds A-22.001 to A-22.004 of formula I wherein R₁ is ethyl, and A and R₉ are as defined in Table X, and Q is taken from the group of formula Q₇ as

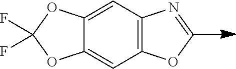 [SMILES: FC1(F)Oc2cc3ncoc3cc2O1]

The Tables B-1 to B-4 below further illustrate specific compounds of the invention.

Table B-1 provides 4 compounds B-1.001 to B-1.004 of formula I wherein R₁ is —CH₂cyclopropyl, and A and R₉ are as defined in Table X, and Q is taken from the group of formula Q₆ as

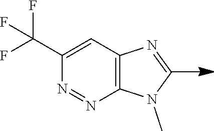

Table B-2 provides 4 compounds B-2.001 to B-2.004 of formula I wherein R₁ is —CH₂cyclopropyl, and A and R₉ are as defined in Table X, and Q is taken from the group of formula Q₁ as

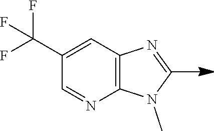

Table B-3 provides 4 compounds B-3.001 to B-3.004 of formula I wherein R₁ is —CH₂cyclopropyl, and A and R₉ are as defined in Table X, and Q is taken from the group of formula Q₂ as

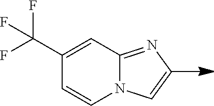 [SMILES: FC(F)(F)c1ccn2ccnc2c1]

Table B-4 provides 4 compounds B-4.001 to B-4.004 of formula I wherein R₁ is —CH₂cyclopropyl, and A and R₉ are as defined in Table X, and Q is taken from the group of formula Q₁ as

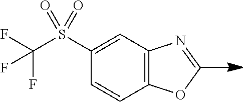 [SMILES: O=S(=O)(c1ccc2ocnc2c1)C(F)(F)F]

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina, nematodes or molluscs. The insecticidal, nematicidal, molluscicidal or acaricidal activity of the active ingredients according to the invention can manifest itself directly, i. e. in mortality or destruction of the pests, which takes place either immediately or only after some time has elapsed, for example during ecdysis, or indirectly, for example in a reduced oviposition and/or hatching rate, anti-feedant effect, and/or growth inhibition.

Compounds of formula (I) according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile, improved physico-chemical properties, or increased biodegradability or environmental profile). In particular, it has been surprisingly found that certain compounds of formula (I) show an advantageous safety profile with respect to non-target organisms, for example, non-target arthropods, in particular pollinators such as honey bees, solitary bees, and bumble bees. Most particularly, Apis mellifera.

In this regard, certain compounds of formula (I) of the invention can be distinguished from known compounds by virtue of greater efficacy at low application rates, which can be verified by the person skilled in the art using experimental procedures similar to or adapted from those outlined in the biological examples, using lower application rates if necessary, for example 50 ppm, 12.5 ppm, 6 ppm, 3 ppm, 1.5 ppm, 0.8 ppm or 0.2 ppm.

Further it has surprisingly found that that compounds of formula (I) of the invention show advantageous physico-chemical properties for application in crop protection, in particular reduced melting point, reduced lipophilicity and increased water solubility. Such properties have been found to be advantageous for plant uptake and systemic distribution, see for example A. Buchholz, S. Trapp, Pest Manag Sci 2016; 72: 929-939) in order to control certain pest species named below.

Putative metabolites of the compounds of the formula I which may be formed in the practice of the invention in conjunction with one or more of the methods, pests, crops and/or targets described below include the amide compounds of formula I-M1 and the acid compounds of formula I-M2, each corresponding to a parent nitrile compound of formula I:

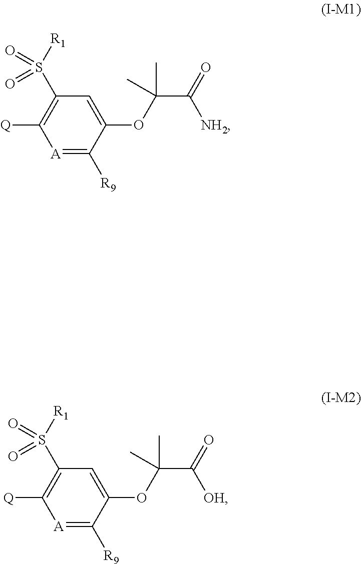

wherein Q, R₁, R₂, R₃, R₄, R₉, X₁, G₁, G₂, and A are as defined under formula I above, or an agrochemically acceptable salt, stereoisomer, enantiomer, tautomer or N-oxide thereof. Among the specific putative metabolites there may be mentioned: (1) an amide compound of formula I-M1 that corresponds to a parent nitrile selected from the group consisting of the compounds described in Tables A-1 to A-22 and B-1 to B-4 and Table P; and (2) an acid compound of formula I-M2 that corresponds to a parent nitrile selected from the group consisting of the compounds described in Tables A-1 to A-22 and B-1 to B-4 and Table P.

Examples of the abovementioned animal pests are:

from the order Acarina, for example,

Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;

from the order Anoplura, for example,

Haematopinus spp., Linognathus spp., Pediculus spp., Pemphigus spp. and Phylloxera spp.;

from the order Coleoptera, for example,

Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;

from the order Diptera, for example,

Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;

from the order Hemiptera, for example,

Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euschistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens;

Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;

from the order Hymenoptera, for example,

Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;

from the order Isoptera, for example,

Coptotermes spp, Corniternes cumulans, Incisitermes spp, Macrotermes spp, Mastotermes spp, Microtermes spp, Reticulitermes spp.; Solenopsis geminate

from the order Lepidoptera, for example,

Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;

from the order Mallophaga, for example,

Damalinea spp. and Trichodectes spp.;

from the order Orthoptera, for example,

Blatta spp., Blattella spp., Gryllotalpa spp., Leucophaea maderae, Locusta spp., Neocurtilla hexadactyla, Periplaneta spp., Scapteriscus spp, and Schistocerca spp.;

from the order Psocoptera, for example,

Liposcelis spp.;

from the order Siphonaptera, for example,

Ceratophyllus spp., Ctenocephalides spp. and Xenopsylla cheopis;

from the order Thysanoptera, for example,

Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;

from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp orjute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1 Fa2, Cry2Ab, Cry3A, Cry3Bb1 or Cry9C, or vegetative insecticidal proteins (Vip), e.g. Vip1, Vip2, Vip3 or Vip3A; or

insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603 x MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern hophornbeam, |
| | | Dogwood, Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests:

beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively).

Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance.

Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium | — | 6% | 10% |
| diisobutylnaphthalenesulfonate | | | |
| phenol polyethylene glycol | — | 2% | — |
| ether (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry | | | |
|---|---|---|---|
| seed treatment | a) | b) | c) |
| active ingredients | 25% | 50% | 75% |
| light mineral oil | 5% | 5% | 5% |
| highly dispersed | 5% | 5% | — |
| silicic acid | | | |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol | 3% |
| ether (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol | 3% |
| (mol. wt. 200) | |
| Kaolin | 89% |

The finely ground combination is uniformly applied, in a mixer, to the kaolin moistened with polyethylene glycol. Non-dusty coated granules are obtained in this manner.

Suspension Concentrate

| | |
|---|---|
| active ingredients | 40% |
| propylene glycol | 10% |
| nonylphenol polyethylene glycol | 6% |
| ether (15 mol of ethylene oxide) | |
| Sodium lignosulfonate | 10% |
| carboxymethylcellulose | 1% |
| silicone oil (in the form of a | 1% |
| 75% emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with | 2% |
| 10-20 moles EO | |
| 1,2-benzisothiazolin-3-one | 0.5% |
| (in the form of a 20% | |
| solution in water) | |
| monoazo-pigment calcium salt | 5% |
| Silicone oil (in the form of a | 0.2% |
| 75% emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

LCMS and GCMS Methods:

Method 1:

Spectra were recorded on a Mass Spectrometer from Waters (ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B, 100% A; 1.2-1.5 min 100% B; Flow (ml/min) 0.85.

Method 2:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH; gradient: 0 min 0% B, 100% A; 2.7-3.0 min 100% B; Flow (ml/min) 0.85.

Method 3:

Spectra were recorded on a Mass Spectrometer from Agilent Technologies (6410 Triple Quadruple Mass Spectrometer) equipped with an electrospray source (Polarity: Positive and Negative Polarity Switch, Capillary: 4.00 kV, Fragmentor: 100.00 V, Gas Temperature: 350° C., Gas Flow: 11 L/min, Nebulizer Gas: 45 psi, Mass range: 110-1000 Da, DAD Wavelength range: 210-400 nm). Column: KINETEX EVO C18, length 50 mm, diameter 4.6 mm, particle size 2.6 μm. Column oven temperature 40° C. Solvent gradient: A=Water with 0.1% formic acid:Acetonitrile (95:5 v/v). B=Acetonitrile with 0.1% formic acid. Gradient=0 min 90% A, 10% B; 0.9-1.8 min 0% A, 100% B, 2.2-2.5 min 90% A, 10% B. Flow rate 1.8 mL/min.

Method 4:

Spectra were recorded on a Mass Spectrometer from Waters (Acquity SDS Mass Spectrometer) equipped with an electrospray source (Polarity: Positive and Negative Polarity Switch, Capillary: 3.00 kV, Cone Voltage: 41.00 V, Source temperature: 150° C., Desolvation Gas Flow: 1000 L/Hr, Desolvation temperature: 500° C., Gas Flow @Cone: 50 L/hr, Mass range: 110-800 Da, PDA wavelength range: 210-400 nm. Column: Acquity UPLC HSS T3 C18, length 30 mm, diameter 2.1 mm, particle size 1.8 μm. Column oven temperature 40° C. Solvent gradient: A=Water with 0.1% formic acid:Acetonitrile (95:5 v/v). B=Acetonitrile with 0.05% formic acid. Gradient=0 min 90% A, 10% B; 0.2 min 50% A, 50% B; 0.7-1.3 min 0% A, 100% B; 1.4-1.6 min 90% A, 10% B. Flow rate 0.8 mL/min.

Method 5:

Spectra were recorded on a Mass Spectrometer from Waters (SQ detector 2 single quadrupole mass spectrometer) equipped with an electrospray source (Polarity: positive or negative ions, Capillary: 2.50 kV, Cone voltage: 41 V, Extractor: 3.00 V, Source Temperature: 150° C., Desolvation Temperature: 500° C., Cone Gas Flow: 50 L/Hr, Desolvation Gas Flow: 1000 L/Hr, Mass range: 100 to 600 Da) and an Acquity UPLC from Waters: Quaternary pump, heated column compartment and diode-array detector. Column used Waters UPLC HSS T3, 1.8 μm, 30×2.1 mm. Column oven temperature 40° C. DAD Wavelength range (nm): 200 to 350. Solvent Gradient: A=water+5% Acetonitrile+0.05% HCOOH, B=Acetonitrile+0.05% HCOOH. Gradient=0 min 90% A, 10% B; 0.2 min 50% A, 50% B; 0.7-1.3 min 0% A, 100% B; 1.4-1.6 min 90% A, 10% B. Flow rate 0.6 mL/min.

### Preparation of Examples of Compounds of Formula (I)

### Example P1: Preparation of 2-[[5-(cyclopropylmethylsulfonyl)-6-[7-methyl-3-(trifluoromethyl)imidazo[4,5-c]pyridazin-6-yl]-3-pyridyl]oxy]-2-methyl-propanenitrile (Compound P1)

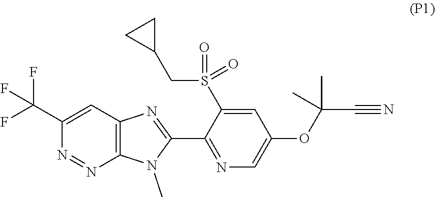 [SMILES: Cn1c(-c2ncc(OC(C)(C)C#N)cc2S(=O)(=O)CC2CC2)nc2cc(C(F)(F)F)nnc21]

### Step 1: Preparation of 2-[[5-(cyclopropylmethylsulfanyl)-6-[7-methyl-3-(trifluoromethyl)imidazo[4,5-c]pyridazin-6-yl]-3-pyridyl]oxy]-2-methyl-propanamide (Compound I8)

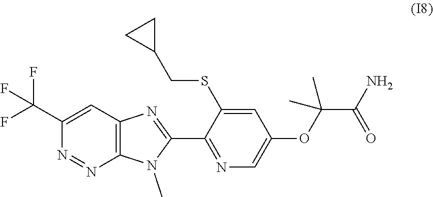 [SMILES: Cn1c(-c2ncc(OC(C)(C)C(N)=O)cc2SCC2CC2)nc2cc(C(F)(F)F)nnc21]

5-(Cyclopropylmethylsulfanyl)-6-[7-methyl-3-(trifluoromethyl) imidazo[4,5-c]pyridazin-6-yl]pyridin-3-ol (compound I7 prepared in analogy to step 1 of EXAMPLE P2) was treated under the same conditions described in step 2 of EXAMPLE P2 to give the desired compound.

LCMS (method 5): m/z 467 [M+H]⁺; retention time: 1.13 min.

### Step 2: Preparation of 2-[[5-(cyclopropylmethylsulfanyl)-6-[7-methyl-3-(trifluoromethyl)imidazo[4,5-c]pyridazin-6-yl]-3-pyridyl]oxy]-2-methyl-propanenitrile (Compound I9)

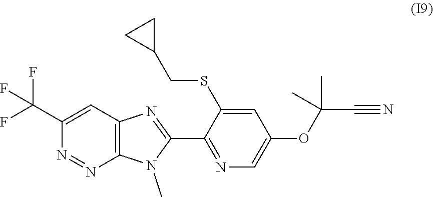 [SMILES: Cn1c(-c2ncc(OC(C)(C)C#N)cc2SCC2CC2)nc2cc(C(F)(F)F)nnc21]

2-[[5-(Cyclopropylmethylsulfanyl)-6-[7-methyl-3-(trifluoromethyl)imidazo[4,5-c]pyridazin-6-yl]-3-pyridyl]oxy]-2-methyl-propanamide (compound I8 prepared as described above) was treated under the same conditions described in step 3 of EXAMPLE P2 to give the desired compound.

LCMS (method 5): m/z 449 [M+H]⁺; retention time: 1.21 min.

### Step 3: Preparation of 2-[[5-(cyclopropylmethylsulfonyl)-6-[7-methyl-3-(trifluoromethyl)imidazo[4,5-c]pyridazin-6-yl]-3-pyridyl]oxy]-2-methyl-propanenitrile (Compound P1)

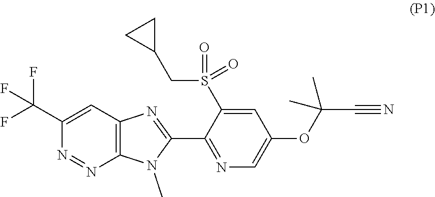 [SMILES: Cn1c(-c2ncc(OC(C)(C)C#N)cc2S(=O)(=O)CC2CC2)nc2cc(C(F)(F)F)nnc21]

2-[[5-(Cyclopropylmethylsulfanyl)-6-[7-methyl-3-(trifluoromethyl)imidazo[4,5-c]pyridazin-6-yl]-3-pyridyl]oxy]-2-methyl-propanenitrile (compound I9 prepared as described above) was treated under the same conditions described in step 4 of EXAMPLE P2 to give the desired compound.

LCMS (method 5): m/z 481 [M+H]⁺; retention time: 1.17 min.

### Example P2: Preparation of 2-[[5-ethylsulfonyl-6-[7-methyl-3-(trifluoromethyl)imidazo[4,5-c]pyridazin-6-yl]-3-pyridyl]oxy]-2-methyl-propanenitrile (Compound P2)

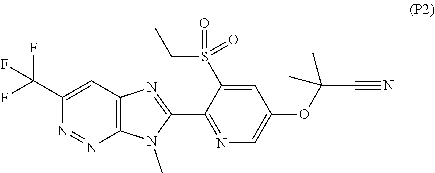 [SMILES: CCS(=O)(=O)c1cc(OC(C)(C)C#N)cnc1-c1nc2cc(C(F)(F)F)nnc2n1C]

### Step 1: Preparation of 5-ethylsulfanyl-6-[7-methyl-3-(trifluoromethyl)imidazo[4,5-c]pyridazin-6-yl]pyridin-3-ol (Compound I1)

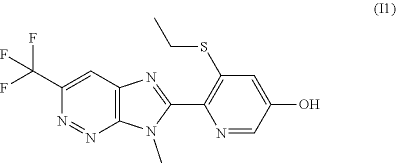 [SMILES: CCSc1cc(O)cnc1-c1nc2cc(C(F)(F)F)nnc2n1C]

Cesium carbonate (19.5 g, 59.8 mmol, 2.50 equiv.) and (E)-benzaldehyde oxime (3.4 mL, 31.1 mmol, 1.30 equiv.) were added to a solution of 6-(5-bromo-3-ethylsulfanyl-2-pyridyl)-7-methyl-3-(trifluoromethyl)imidazo[4,5-c]pyridazine (prepared according to WO 2016059145) (10.0 g, 23.9 mmol) in acetonitrile (240 mL). The resulting suspension was stirred at 50° C. for 42 hours. After cooling down to room temperature, the reaction mixture was concentrated under reduced pressure, the crude residue was partioned between ethyl acetate and water, and the pH of the aqueous phase was adjusted to 1-2 by addition of a 1N hydrochloric acid solution. The aqueous phase was extracted twice with ethyl acetate, the combined organic phases were dried over sodium sulfate, filtered and concentrated. Purification of the crude material by flash chromatography over silica gel (0-10% methanol in dichloromethane) afforded the desired product as a yellow solid (6.90 g, 19.0 mmol).

¹H NMR (400 MHz, dimethylsulfoxide-d6) δ ppm 1.25 (t, J=7.34 Hz, 3H) 2.99 (q, J=7.34 Hz, 2H) 4.13 (s, 3H) 7.38 (d, J=2.20 Hz, 1H) 8.17 (d, J=2.20 Hz, 1H) 8.55 (s, 1H) 10.94 (s, 1H).

### Step 2: Preparation of 2-[[5-ethylsulfanyl-6-[7-methyl-3-(trifluoromethyl)imidazo[4,5-c]pyridazin-6-yl]-3-pyridyl]oxy]-2-methyl-propanamide (Compound I2)

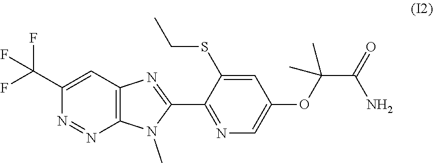 [SMILES: CCSc1cc(OC(C)(C)C(N)=O)cnc1-c1nc2cc(C(F)(F)F)nnc2n1C]

Cesium carbonate (303 mg, 0.93 mmol, 1.10 equiv.) was added to a solution of 5-ethylsulfanyl-6-[7-methyl-3-(trifluoromethyl)imidazo[4,5-c]pyridazin-6-yl]pyridin-3-ol (compound I1 prepared as described above) (300 mg, 0.84 mmol) in acetonitrile (8.4 mL). The resulting suspension was stirred for 5 min before adding 2-bromo-2-methyl-propanamide (294 mg, 1.77 mmol, 2.10 equiv.), and the reaction mixture was heated and stirred overnight at 70° C. After cooling down to room temperature, the reaction mixture was concentrated under reduced pressure, the crude residue was partioned between ethyl acetate and water, and the pH of the aqueous phase was adjusted to 1 by addition of a 1N hydrochloric acid solution. The aqueous phase was extracted three times with ethyl acetate and once with dichloromethane, the combined organic phases were dried over sodium sulfate, filtered and concentrated. Purification of the crude material by flash chromatography over silica gel (0-10% methanol in dichloromethane) afforded the desired product as a yellow solid (156 mg, 0.56 mmol).

¹H NMR (400 MHz, dimethylsulfoxide-d6) δ ppm 1.26 (t, J=7.34 Hz, 3H) 1.60 (s, 6H) 2.96 (q, J=7.34 Hz, 2H) 4.15 (s, 3H) 7.30 (s broad, 1H) 7.41 (d, J=2.20 Hz, 1H) 7.49 (m, 1H) 8.23 (d, J=2.20 Hz, 1H) 8.69 (s, 1H).

### Step 3: Preparation of 2-[[5-ethylsulfanyl-6-[7-methyl-3-(trifluoromethyl)imidazo[4,5-c]pyridazin-6-yl]-3-pyridyl]oxy]-2-methyl-propanenitrile (Compound I3)

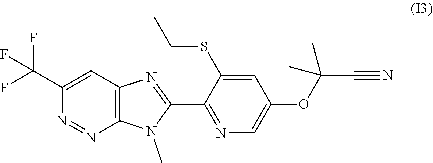 [SMILES: CCSc1cc(OC(C)(C)C#N)cnc1-c1nc2cc(C(F)(F)F)nnc2n1C]

Trifluoroacetic anhydride (182 μL, 1.30 mmol, 3.00 equiv.) was added at 0° C. to a solution of 2-[[5-ethylsulfanyl-6-[7-methyl-3-(trifluoromethyl)imidazo[4,5-c]pyridazin-6-yl]-3-pyridyl]oxy]-2-methyl-propanamide (compound I2 prepared as described above) (317 mg, 0.43 mmol) in dichloromethane (4.30 mL) with triethylamine (243 μL, 1.73 mmol, 4.00 equiv.). After stirring overnight at room temperature, another addition of trifluoroacetic anhydride (182 μL, 1.30 mmol, 3.00 equiv.) and triethylamine (243 μL, 1.73 mmol, 4.00 equiv.) was done and the reaction mixture stirred further at room temperature for 2 hours. The reaction mixture was carefully quenched by adding methanol followed by a saturated sodium hydrogenocarbonate solution. The aqueous phase was extracted twice with dichloromethane, the combined organic layers were dried over sodium sulfate, filtered and concentrated. The crude material was purified by flash chromatography over silica gel (0-10% methanol in dichloromethane) to give the desired product as a yellow oil (156 mg, 0.37 mmol).

¹H NMR (400 MHz, chloroform-d) δ ppm 1.42 (t, J=7.34 Hz, 3H) 1.88 (s, 6H) 3.03 (q, J=7.34 Hz, 2H) 4.31 (s, 3H) 7.72 (d, J=2.57 Hz, 1H) 8.26 (s, 1H) 8.39 (d, J=2.57 Hz, 1H).

### Step 4: Preparation of 2-[[5-ethylsulfonyl-6-[7-methyl-3-(trifluoromethyl)imidazo[4,5-c]pyridazin-6-yl]-3-pyridyl]oxy]-2-methyl-propanenitrile (Compound P2)

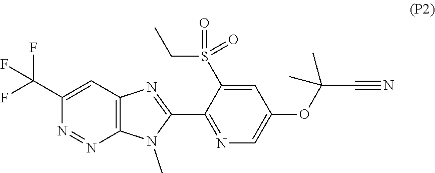 [SMILES: CCS(=O)(=O)c1cc(OC(C)(C)C#N)cnc1-c1nc2cc(C(F)(F)F)nnc2n1C]

To a solution of 2-[[5-ethylsulfanyl-6-[7-methyl-3-(trifluoromethyl)imidazo[4,5-c]pyridazin-6-yl]-3-pyridyl]oxy]-2-methyl-propanenitrile (compound I3 prepared as described above) (156 mg, 0.37 mmol) in dichloromethane (3.12 mL) at 0° C. was added 3-chlorobenzenecarboperoxoic acid (191.2 mg, 0.776 mmol) and the mixture stirred at 0° C. for 30 minutes, then at room temperature overnight. The reaction mixture was quenched with aqueous solutions of sodium hydroxide (1N, 5 mL) and sodium thiosulfate (5 mL). The aqueous layer was extracted 3 times with dichloromethane, the combined organic layers washed twice with 1N aqueous sodium hydroxide, brine, dried over sodium sulfate, filtered and evaporated in vacuo. The crude material was triturated in cyclohexane, the formed precipitate filtered and dried to afford the desired product. Alternatively, the crude material may be purified by flash chromatography over silica gel.

LCMS (method 1): m/z 455 [M+H]⁺; retention time: 0.98 min.

### Example P3: Preparation of 2-[[5-ethylsulfonyl-6-[3-methyl-6-(trifluoromethyl)imidazo[4,5-b]pyridin-2-yl]-3-pyridyl]oxy]-2-methyl-propanenitrile (Compound P3)

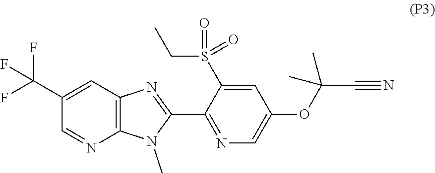 [SMILES: CCS(=O)(=O)c1cc(OC(C)(C)C#N)cnc1-c1nc2cc(C(F)(F)F)cnc2n1C]

### Step 1: Preparation of 5-ethylsulfanyl-6-[3-methyl-6-(trifluoromethyl)imidazo[4,5-b]pyridin-2-yl]pyridin-3-ol (Compound I4)

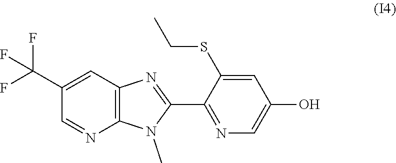 [SMILES: CCSc1cc(O)cnc1-c1nc2cc(C(F)(F)F)cnc2n1C]

Cesium carbonate (12.9 g, 39.5 mmol, 2.20 equiv.) and (E)-benzaldehyde oxime (2.55 mL, 23.4 mmol, 1.30 equiv.) were added to a solution of 2-(5-bromo-3-ethylsulfanyl-2-pyridyl)-3-methyl-6-(trifluoromethyl)imidazo[4,5-b]pyridine (CAS 1421955-74-9) (7.50 g, 18.0 mmol) in N,N-dimethylformamide (36 mL). The resulting suspension was stirred at 80° C. overnight. After cooling down to room temperature, the reaction mixture was diluted with dichloromethane (500 mL), the organic phase was washed with water (3*200 mL) and the pH of the aqueous phase was adjusted to 1-2 by addition of a 1N hydrochloric acid solution. The aqueous phase was extracted with dichloromethane (5*300 mL), the combined organic phases were dried over sodium sulfate, filtered and concentrated. Purification of the crude material by flash chromatography over silica gel (ethyl acetate in cyclohexane) afforded the desired product (5.80 g, 16.4 mmol).

LCMS (method 1): m/z 355 [M+H]⁺; retention time: 0.94 min.

### Step 2: Preparation of 2-[[5-ethylsulfanyl-6-[3-methyl-6-(trifluoromethyl)imidazo[4,5-b]pyridin-2-yl]-3-pyridyl]oxy]acetonitrile (Compound I5)

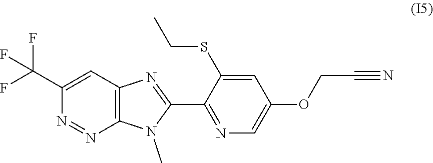 [SMILES: CCSc1cc(OCC#N)cnc1-c1nc2cc(C(F)(F)F)nnc2n1C]

Potassium carbonate (1.21 g, 8.47 mmol, 1.50 equiv.) followed by bromoacetonitrile (608 μL, 8.47 mmol, 1.50 equiv.) were added at room temperature to a solution of 5-ethylsulfanyl-6-[3-methyl-6-(trifluoromethyl)imidazo[4,5-b]pyridin-2-yl]pyridin-3-ol (compound I4 prepared as described above) (2.00 g, 5.64 mmol) in N,N-dimethylformamide (40 mL) under argon. After stirring for 5 hours, the reaction mixture was poured over water (300 mL), and the aqueous phase was extracted twice with ethyl acetate (300 mL). The combined organic phases were washed with water (3*200 mL), dried over sodium sulfate, filtered and concentrated. The crude material was purified by chromatography over silica gel (ethyl acetate in cyclohexane) to give the desired compound as a yellow solid (2.08 g, 5.28 mmol).

LCMS (method 1): m/z 394 [M+H]⁺; retention time: 1.01 min.

### Step 3: Preparation of 2-[[5-ethylsulfanyl-6-[3-methyl-6-(trifluoromethyl)imidazo[4,5-b]pyridin-2-yl]-3-pyridyl]oxy]-2-methyl-propanenitrile (Compound I6)

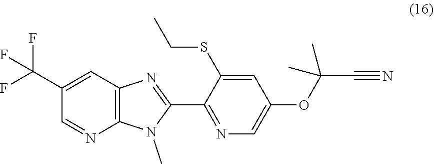 [SMILES: CCSc1cc(OC(C)(C)C#N)cnc1-c1nc2cc(C(F)(F)F)cnc2n1C]

A 1M lithium hexamethyldisilazane solution in tetrahydrofuran (15.8 mL, 15.8 mmol, 3.00 equiv.) was added dropwise via a dropping funnel to a solution of 2-[[5-ethylsulfanyl-6-[3-methyl-6-(trifluoromethyl)imidazo[4,5-b]pyridin-2-yl]-3-pyridyl]oxy]acetonitrile (compound I5 prepared as described above) (2.07 g, 5.26 mmol) and methyl iodide (1.31 mL, 21.0 mmol, 4.00 equiv.) in tetrahydrofuran (32 mL) cooled at 0° C. After complete addition, the reaction mixture was stirred for 1 hour with the ice bath then warmed up to room temperature and stirred overnight. The reaction mixture was quenched by pouring over a saturated sodium hydrogenocarbonate aqueous solution at 0° C. (50 mL). The aqueous phase was extracted with ethyl acetate (2*50 mL). The combined organic phases were dried over sodium sulfate, filtered and evaporated. The crude material was purified by flash chromatography over silica gel (ethyl acetate in cyclohexane) to afford the desired compound (700 mg, 1.66 mmol).

LCMS (method 1): m/z 422 [M+H]⁺; retention time: 1.11 min.

### Step 4: Preparation of 2-[[5-ethylsulfonyl-6-[3-methyl-6-(trifluoromethyl)imidazo[4,5-b]pyridin-2-yl]-3-pyridyl]oxy]-2-methyl-propanenitrile (Compound P3)

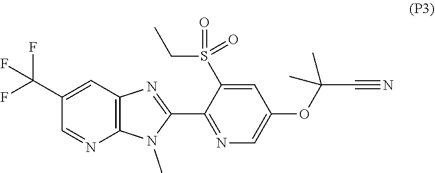 [SMILES: CCS(=O)(=O)c1cc(OC(C)(C)C#N)cnc1-c1nc2cc(C(F)(F)F)cnc2n1C]

2-[[5-Ethylsulfanyl-6-[3-methyl-6-(trifluoromethyl)imidazo[4,5-b]pyridin-2-yl]-3-pyridyl]oxy]-2-methyl-propanenitrile (compound I6 prepared as described above) was treated under the same conditions described in step 4 of EXAMPLE P2 to give the desired compound.

LCMS (method 1): m/z 454 [M+H]⁺; retention time: 1.05 min.

### Example P5: Preparation of 2-[[5-ethylsulfonyl-6-[5-methoxy-3-methyl-4-oxo-6-(trifluoromethyl)imidazo[4,5-c]pyridin-2-yl]-3-pyridyl]oxy]-2-methyl-propanenitrile (Compound P5)

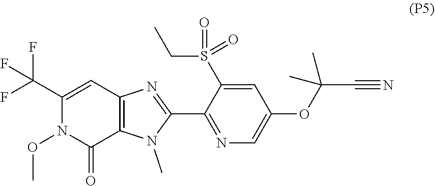 [SMILES: CCS(=O)(=O)c1cc(OC(C)(C)C#N)cnc1-c1nc2cc(C(F)(F)F)n(OC)c(=O)c2n1C]

2-[[5-Ethylsulfanyl-6-[5-methoxy-3-methyl-4-oxo-6-(trifluoromethyl)imidazo[4,5-c]pyridin-2-yl]-3-pyridyl]oxy]-2-methyl-propanenitrile (compound I10) was treated under the same conditions described in step 4 of EXAMPLE P2 to give the desired compound.

LCMS (method 5): m/z 500 [M+H]⁺; retention time: 1.02 min.

### Example P4: Preparation of 2-[[5-ethylsulfonyl-2-methyl-6-[3-methyl-6-(trifluoromethyl)imidazo[4,5-b]pyridin-2-yl]-3-pyridyl]oxy]-2-methyl-propanenitrile (Compound P4)

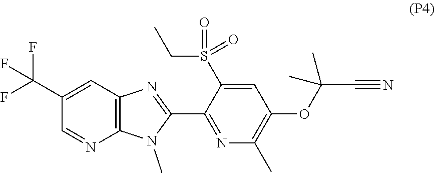 [SMILES: CCS(=O)(=O)c1cc(OC(C)(C)C#N)c(C)nc1-c1nc2cc(C(F)(F)F)cnc2n1C]

### Step 1: Preparation of 5-ethylsulfanyl-2-iodo-6-[3-methyl-6-(trifluoromethyl)imidazo[4,5-b]pyridin-2-yl]pyridin-3-ol (Compound I13)

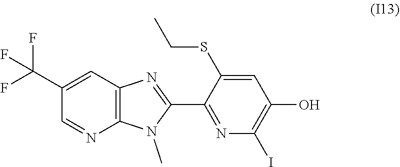 [SMILES: CCSc1cc(O)c(I)nc1-c1nc2cc(C(F)(F)F)cnc2n1C]

Molecular iodine (8.69 g, 34.2 mmol) was added in portions to a mixture of 5-ethylsulfanyl-6-[3-methyl-6-(trifluoromethyl)imidazo[4,5-b]pyridin-2-yl]pyridin-3-ol (compound I4 prepared as described in step 1 of example P3) (10.1 g, 28.5 mmol) and sodium carbonate (6.34 g, 59.8 mmol) in water (85.5 mL) and acetonitrile (85.5 mL) at room temperature under argon. After stirring for 3 hours, the reaction mixture was quenched with a 10% w/w sodium thiosulfate aqueous solution and then extracted with ethyl acetate. The combined organic phases were washed with brine, dried over sodium sulfate, filtered and concentrated to afford the desired product. This material was used as such in the next step.

LCMS (method 1): m/z 481 [M+H]⁺; retention time: 1.06 min.

### Step 2: Preparation of 5-ethylsulfanyl-2-methyl-6-[3-methyl-6-(trifluoromethyl)imidazo[4,5-b]pyridin-2-yl]pyridin-3-ol (Compound I14)

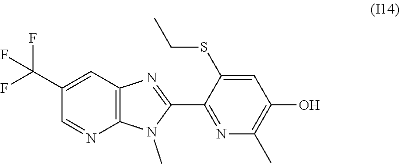 [SMILES: CCSc1cc(O)c(C)nc1-c1nc2cc(C(F)(F)F)cnc2n1C]

Trimethylboroxine (10.4 mL, 73.49 mmol) was added to a mixture of 5-ethylsulfanyl-2-iodo-6-[3-methyl-6-(trifluoromethyl)imidazo[4,5-b]pyridin-2-yl]pyridin-3-ol (compound I13 prepared as described above) (14.12 g, 29.39 mmol), potassium carbonate (12.83 g, 88.18 mmol) and [1,1′-bis(diphenyl-phosphino)ferrocene]palladium(II) dichloride dichloromethane complex (6.05 g, 7.42 mmol) in 1,4-dioxane (147 mL) at room temperature under argon. The reaction mixture was heated to 100° C. and stirred for 3 hours. After cooling to room temperature, the crude mixture was filtered over a pad of celite and the residue washed with ethyl acetate. The filtrate was concentrated under vacuum to give the crude product, which was purified by flash chromatography over silica gel (ethyl acetate in cyclohexane) to afford the desired product.

LCMS (method 1): m/z 369 [M+H]⁺; retention time: 0.96 min.

### Step 3: Preparation of 2-[[5-ethylsulfanyl-2-methyl-6-[3-methyl-6-(trifluoromethyl)imidazo[4,5-b]pyridin-2-yl]-3-pyridyl]oxy]acetonitrile (Compound I15)

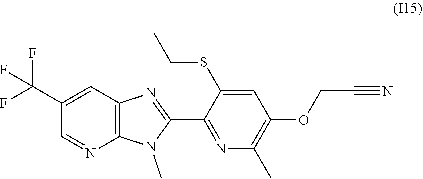 [SMILES: CCSc1cc(OCC#N)c(C)nc1-c1nc2cc(C(F)(F)F)cnc2n1C]

5-Ethylsulfanyl-2-methyl-6-[3-methyl-6-(trifluoromethyl)imidazo[4,5-b]pyridin-2-yl]pyridin-3-ol (compound I14 prepared as described above) was treated under the same conditions described in step 2 of EXAMPLE P3 to give the desired compound.

LCMS (method 1): m/z 408 [M+H]⁺; retention time: 1.05 min.

### Step 4: Preparation of 2-[[5-ethylsulfanyl-2-methyl-6-[3-methyl-6-(trifluoromethyl)imidazo[4,5-b]pyridin-2-yl]-3-pyridyl]oxy]-2-methyl-propanenitrile (Compound I16)

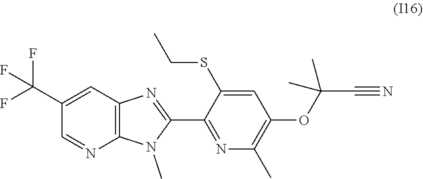 [SMILES: CCSc1cc(OC(C)(C)C#N)c(C)nc1-c1nc2cc(C(F)(F)F)cnc2n1C]

2-[[5-ethylsulfanyl-2-methyl-6-[3-methyl-6-(trifluoromethyl)imidazo[4,5-b]pyridin-2-yl]-3-pyridyl]oxy]acetonitrile (compound I15 prepared as described above) was treated under the same conditions described in step 3 of EXAMPLE P3 to give the desired compound.

LCMS (method 1): m/z 436 [M+H]⁺; retention time: 1.16 min.

### Step 5: Preparation of 2-[[5-ethylsulfonyl-2-methyl-6-[3-methyl-6-(trifluoromethyl)imidazo[4,5-b]pyridin-2-yl]-3-pyridyl]oxy]-2-methyl-propanenitrile (Compound P4)

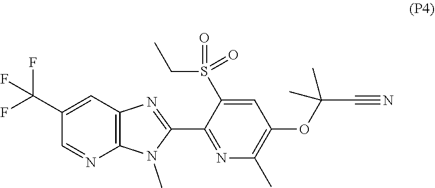 [SMILES: CCS(=O)(=O)c1cc(OC(C)(C)C#N)c(C)nc1-c1nc2cc(C(F)(F)F)cnc2n1C]

2-[[5-ethylsulfanyl-2-methyl-6-[3-methyl-6-(trifluoromethyl)imidazo[4,5-b]pyridin-2-yl]-3-pyridyl]oxy]-2-methyl-propanenitrile (compound I16 prepared as described above) was treated under the same conditions described in step 4 of EXAMPLE P2 to give the desired compound.

LCMS (method 1): m/z 468 [M+H]⁺; retention time: 1.07 min.

### Example P7: Preparation of 2-[[6-[5-cyclopropyl-3-methyl-4-oxo-6-(trifluoromethyl)imidazo[4,5-c]pyridin-2-yl]-5-ethylsulfonyl-3-pyridyl]oxy]-2-methyl-propanenitrile (Compound P7)

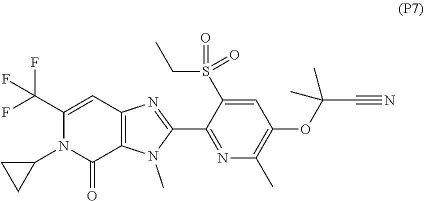 [SMILES: CCS(=O)(=O)c1cc(OC(C)(C)C#N)c(C)nc1-c1nc2cc(C(F)(F)F)n(C3CC3)c(=O)c2n1C]

### Step 1: Preparation of 5-cyclopropyl-2-(3-ethylsulfanyl-5-hydroxy-2-pyridyl)-3-methyl-6-(trifluoromethyl)imidazo[4,5-c]pyridin-4-one (Compound I17)

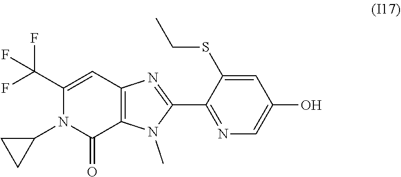 [SMILES: CCSc1cc(O)cnc1-c1nc2cc(C(F)(F)F)n(C3CC3)c(=O)c2n1C]

Cesium carbonate (2.75 g, 8.43 mmol, 3.00 equiv.) and (E)-benzaldehyde oxime (614 μL, 5.62 mmol, 2.00 equiv.) were added to a solution of 2-(5-bromo-3-ethylsulfanyl-2-pyridyl)-5-cyclopropyl-3-methyl-6-(trifluoromethyl)imidazo[4,5-c]pyridin-4-one (prepared as described in WO 2017089190) (1.33 g, 2.81 mmol) in N,N-dimethylformamide (12 mL). The resulting suspension was stirred at 45° C. overnight. After cooling down to room temperature, the reaction mixture was diluted with water and the pH of the aqueous phase was adjusted to 1 by addition of a 2N hydrochloric acid solution. The aqueous phase was extracted with ethyl acetate, the combined organic phases were dried over sodium sulfate, filtered and concentrated. Purification of the crude material by flash chromatography over silica gel (ethyl acetate in cyclohexane) afforded the desired product as a white solid (1.00 g, 2.44 mmol).

LCMS (method 1): m/z 355 [M+H]⁺; retention time: 0.94 min.

¹H NMR (400 MHz, chloroform-d) δ ppm 1.06 (brs, 2H) 1.18-1.37 (m, 5H) 2.75 (q, J=7.38 Hz, 2H) 3.07-3.16 (m, 1H) 4.04 (s, 3H) 7.06 (d, J=2.45 Hz, 1H) 7.28 (m, 1H) 7.98 (d, J=2.45 Hz, 1H).

### Step 2: Preparation of 2-[[6-[5-cyclopropyl-3-methyl-4-oxo-6-(trifluoromethyl)imidazo[4,5-c]pyridin-2-yl]-5-ethylsulfanyl-3-pyridyl]oxy]acetonitrile (Compound I18)

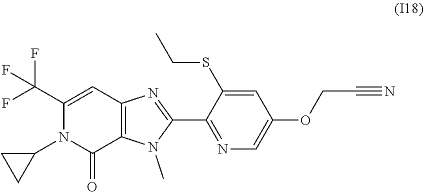 [SMILES: CCSc1cc(OCC#N)cnc1-c1nc2cc(C(F)(F)F)n(C3CC3)c(=O)c2n1C]

Potassium carbonate (404 mg, 2.92 mmol, 1.50 equiv.) followed by bromoacetonitrile after 10 min stirring (177 μL, 2.53 mmol, 1.30 equiv.) were added at 0° C. to a solution of 5-cyclopropyl-2-(3-ethylsulfanyl-5-hydroxy-2-pyridyl)-3-methyl-6-(trifluoromethyl)imidazo[4,5-c]pyridin-4-one (compound I17 prepared as described above) (800 mg, 1.95 mmol) in N,N-dimethylformamide (8.0 mL) under argon. After stirring for 2 hours at room temperature, the reaction mixture was poured over iced water, and the aqueous phase was extracted twice with ethyl acetate. The combined organic phases were washed with water, dried over sodium sulfate, filtered and concentrated. The crude material used directly without further purification.

LCMS (method 3): m/z 450 [M+H]⁺; retention time: 1.48 min.

### Step 3: Preparation of 2-[[6-[5-cyclopropyl-3-methyl-4-oxo-6-(trifluoromethyl)imidazo[4,5-c]pyridin-2-yl]-5-ethylsulfanyl-3-pyridyl]oxy]-2-methyl-propanenitrile (Compound I19)

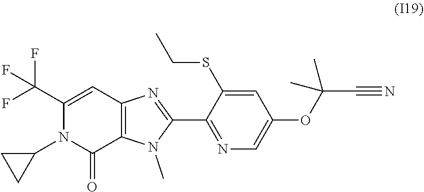 [SMILES: CCSc1cc(OC(C)(C)C#N)cnc1-c1nc2cc(C(F)(F)F)n(C3CC3)c(=O)c2n1C]

A 2M lithium hexamethyldisilazane solution in tetrahydrofuran (2.50 mL, 5.00 mmol, 3.00 equiv.) was added dropwise to a solution of 2-[[6-[5-cyclopropyl-3-methyl-4-oxo-6-(trifluoromethyl)imidazo[4,5-c]pyridin-2-yl]-5-ethylsulfanyl-3-pyridyl]oxy]acetonitrile (compound I18 prepared as described above) (750 mg, 1.67 mmol) and methyl iodide (418 μL, 6.68 mmol, 4.00 equiv.) in tetrahydrofuran (20 mL) cooled at 0° C. The reaction mixture was stirred for 2 hours with the ice bath and then quenched by pouring over a saturated sodium hydrogenocarbonate aqueous solution. The aqueous phase was extracted with ethyl acetate. The combined organic phases were washed with brine, dried over sodium sulfate, filtered and evaporated. The crude material was purified by flash chromatography over silica gel (ethyl acetate in cyclohexane) to afford the desired compound (700 mg, 1.66 mmol).

LCMS (method 3): m/z 478 [M+H]⁺; retention time: 1.54 min.

### Step 4: Preparation of 2-[[6-[5-cyclopropyl-3-methyl-4-oxo-6-(trifluoromethyl)imidazo[4,5-c]pyridin-2-yl]-5-ethylsulfonyl-3-pyridyl]oxy]-2-methyl-propanenitrile (Compound P7)

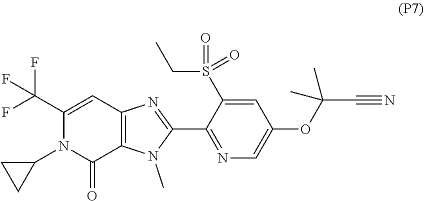 [SMILES: CCS(=O)(=O)c1cc(OC(C)(C)C#N)cnc1-c1nc2cc(C(F)(F)F)n(C3CC3)c(=O)c2n1C]

2-[[6-[5-Cyclopropyl-3-methyl-4-oxo-6-(trifluoromethyl) imidazo[4,5-c]pyridin-2-yl]-5-ethylsulfanyl-3-pyridyl]oxy]-2-methyl-propanenitrile (compound I19 prepared as described above) was treated under the same conditions described in step 4 of EXAMPLE P2 to give the desired compound.

LCMS (method 3): m/z 510 [M+H]⁺; retention time: 1.46 min.

### Example P6: Preparation of 2-[[6-[5-ethyl-3-methyl-4-oxo-6-(trifluoromethyl)imidazo[4,5-c]pyridin-2-yl]-5-ethylsulfonyl-3-pyridyl]oxy]-2-methyl-propanenitrile (Compound P6)

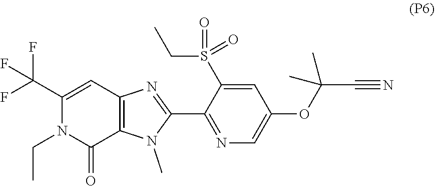 [SMILES: CCn1c(C(F)(F)F)cc2nc(-c3ncc(OC(C)(C)C#N)cc3S(=O)(=O)CC)n(C)c2c1=O]

### Step 1: Preparation of 5-ethyl-2-(3-ethylsulfanyl-5-hydroxy-2-pyridyl)-3-methyl-6-(trifluoromethyl)imidazo[4,5-c]pyridin-4-one (Compound I20)

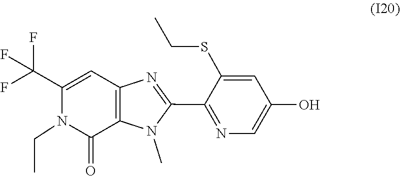 [SMILES: CCSc1cc(O)cnc1-c1nc2cc(C(F)(F)F)n(CC)c(=O)c2n1C]

2-(5-bromo-3-ethylsulfanyl-2-pyridyl)-5-ethyl-3-methyl-6-(trifluoromethyl)imidazo[4,5-c]pyridin-4-one (prepared as described in WO 2017084879) was treated under the same conditions described in step 1 of EXAMPLE P7 to give the desired compound.

LCMS (method 3): m/z 399 [M+H]⁺; retention time: 1.38 min.

¹H NMR (400 MHz, chloroform-d) δ ppm 7.99 (m, 1H) 7.29 (m, 1H) 7.06 (m, 1H) 4.26 (q, J=6.89 Hz, 2H) 4.08 (s, 3H) 2.75 (q, J=7.46 Hz, 2H) 1.42-1.37 (m, 3H) 1.18-1.23 (m, 3H).

### Step 2: Preparation of 2-[[6-[5-ethyl-3-methyl-4-oxo-6-(trifluoromethyl)imidazo[4,5-c]pyridin-2-yl]-5-ethylsulfanyl-3-pyridyl]oxy]acetonitrile (Compound I21)

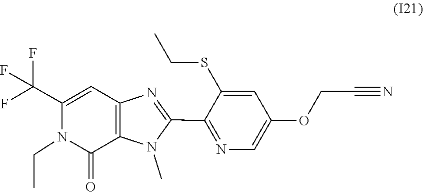 [SMILES: CCSc1cc(OCC#N)cnc1-c1nc2cc(C(F)(F)F)n(CC)c(=O)c2n1C]

5-ethyl-2-(3-ethylsulfanyl-5-hydroxy-2-pyridyl)-3-methyl-6-(trifluoromethyl)imidazo[4,5-c]pyridin-4-one (compound I20 prepared as described above) was treated under the same conditions as described in step 2 of EXAMPLE P7 to give the desired compound.

LCMS (method 4): m/z 438 [M+H]⁺; retention time: 1.01 min.

¹H NMR (400 MHz, chloroform-d) δ ppm 8.25 (m, 1H) 7.33 (m, 1H) 7.31 (m, 1H) 4.93 (m, 2H) 4.28 (m, 2H) 4.20 (m, 3H) 2.95 (m, 2H) 1.41-1.34 (m, 6H).

### Step 3: Preparation of 2-[[6-[5-ethyl-3-methyl-4-oxo-6-(trifluoromethyl)imidazo[4,5-c]pyridin-2-yl]-5-ethylsulfanyl-3-pyridyl]oxy]-2-methyl-propanenitrile (Compound I22)

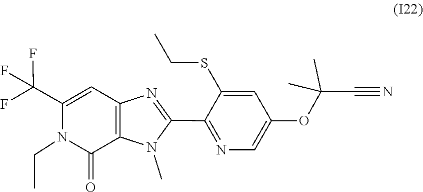 [SMILES: CCSc1cc(OC(C)(C)C#N)cnc1-c1nc2cc(C(F)(F)F)n(CC)c(=O)c2n1C]

2-[[6-[5-ethyl-3-methyl-4-oxo-6-(trifluoromethyl)imidazo[4,5-c]pyridin-2-yl]-5-ethylsulfanyl-3-pyridyl]oxy]acetonitrile (compound I21 prepared as described above) was treated under the same conditions as described in step 3 of EXAMPLE P7 to give the desired compound.

LCMS (method 4): m/z 466 [M+H]⁺; retention time: 1.10 min.

¹H NMR (400 MHz, chloroform-d) δ ppm 8.31 (m, 1H) 7.65 (m, 1H) 7.32 (m, 1H) 4.25 (m, 6H) 2.96 (m, 2H) 1.81-1.84 (m, 6H).

### Step 4: Preparation of 2-[[6-[5-ethyl-3-methyl-4-oxo-6-(trifluoromethyl)imidazo[4,5-c]pyridin-2-yl]-5-ethylsulfonyl-3-pyridyl]oxy]-2-methyl-propanenitrile (Compound P6)

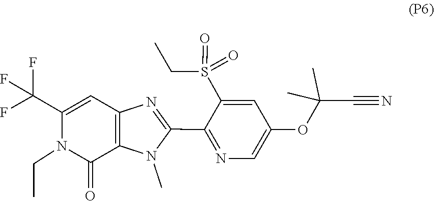 [SMILES: CCn1c(C(F)(F)F)cc2nc(-c3ncc(OC(C)(C)C#N)cc3S(=O)(=O)CC)n(C)c2c1=O]

2-[[6-[5-ethyl-3-methyl-4-oxo-6-(trifluoromethyl)imidazo[4,5-c]pyridin-2-yl]-5-ethylsulfanyl-3-pyridyl]oxy]-2-methyl-propanenitrile (compound I22 prepared as described above) was treated under the same conditions as described in step 4 of EXAMPLE P2 to give the desired compound.

LCMS (method 5): m/z 498 [M+H]⁺; retention time: 1.05 min.

### Example P8: Preparation of 2-[[5-ethylsulfonyl-6-[7-(trifluoromethyl)imidazo[1,2-a]pyridin-2-yl]-3-pyridyl]oxy]-2-methyl-propanenitrile (Compound P8)

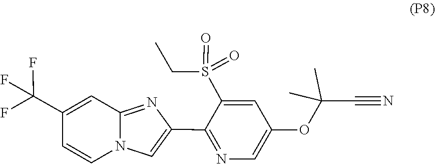 [SMILES: CCS(=O)(=O)c1cc(OC(C)(C)C#N)cnc1-c1cn2ccc(C(F)(F)F)cc2n1]

### Step 1: Preparation of 1-(3-ethylsulfanyl-5-hydroxy-2-pyridyl)ethanone (Compound I23)

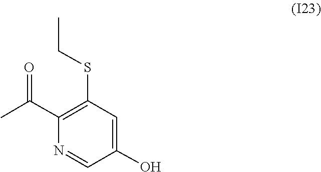

Cesium carbonate (6.65 g, 20.40 mmol, 2.20 equiv.) and (E)-benzaldehyde oxime (1.32 mL, 12.1 mmol, 1.30 equiv.) were added to a solution of 1-(5-chloro-3-ethylsulfanyl-2-pyridyl)ethanone (prepared as described in WO 2016071214) (2.00 g, 9.27 mmol) in N,N-dimethylformamide (18 mL). The resulting suspension was stirred at room temperature overnight. The reaction mixture was diluted with water and the pH of the aqueous phase was adjusted to 1 by addition of a 1N hydrochloric acid solution. The aqueous phase was extracted with ethyl acetate, the combined organic phases were dried over sodium sulfate, filtered and concentrated. Purification of the crude material by flash chromatography over silica gel (ethyl acetate in cyclohexane) afforded the desired product as a white solid (1.47 g, 2.44 mmol).

¹H NMR (400 MHz, dimethylsulfoxide-d6) δ ppm 1.28 (t, J=7.34 Hz, 3H) 2.86 (q, J=7.34 Hz, 2H) 3.33 (s, 3H) 7.15 (d, J=2.20 Hz, 1H) 7.98 (d, J=2.20 Hz, 1H) 10.94 (s br, 1H).

### Step 2: Preparation of 2-[(6-acetyl-5-ethylsulfanyl-3-pyridyl)oxy]-2-methyl-propanamide (Compound I24)

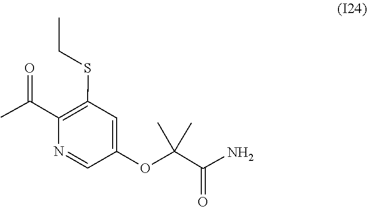 [SMILES: CCSc1cc(OC(C)(C)C(N)=O)cnc1C(C)=O]

Cesium carbonate (9.2 g, 28 mmol, 1.5 equiv.) was added to a solution of 1-(3-ethylsulfanyl-5-hydroxy-2-pyridyl)ethanone (compound I23 prepared as described above) (3.7 g, 19 mmol) in acetonitrile (94 mL). The resulting suspension was stirred for 5 min before adding 2-bromo-2-methyl-propanamide (5.0 g, 30 mmol, 1.6 equiv.), and the reaction mixture was heated and stirred overnight at room temperature. After cooling down to room temperature, the reaction mixture was poured over water, and the aqueous phase was extracted three times with ethyl acetate. The combined organic phases were dried over sodium sulfate, filtered and concentrated. The crude material was used without further purification in the next step.

¹H NMR (400 MHz, dimethylsulfoxide-d6) δ ppm 1.28 (t, J=7.34 Hz, 3H) 1.56 (s, 6H) 1.85 (s, 3H) 2.83 (q, J=7.34 Hz, 2H) 7.15 (d, J=2.20 Hz, 1H) 7.33 (s, 1H) 7.45 (s, 1H) 8.04 (d, J=2.20 Hz, 1H).

### Step 3: Preparation of 2-[(6-acetyl-5-ethylsulfanyl-3-pyridyl)oxy]-2-methyl-propanenitrile (Compound I25)

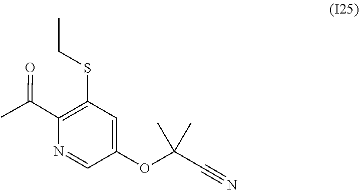 [SMILES: CCSc1cc(OC(C)(C)C#N)cnc1C(C)=O]

Trifluoroacetic anhydride (6.27 mL, 44.6 mmol, 3.00 equiv.) was added at 0° C. to a solution of 2-[(6-acetyl-5-ethylsulfanyl-3-pyridyl)oxy]-2-methyl-propanamide (compound I24 prepared as described above) (6.0 g, 14.9 mmol) in dichloromethane (149 mL) with triethylamine (8.38 mL, 59.5 mmol, 4.00 equiv.). After stirring at room temperature for 2 hours, the reaction mixture was carefully quenched by adding methanol followed by a saturated sodium hydrogenocarbonate solution. The aqueous phase was extracted twice with dichloromethane, the combined organic layers were dried over sodium sulfate, filtered and concentrated. The crude material was purified by flash chromatography over silica gel (0-100% ethyl acetate in cyclohexane) to give the desired product as a yellow oil (3.69 g).

¹H NMR (400 MHz, chloroform-d) δ ppm 1.44 (t, J=7.34 Hz, 3H) 1.83 (s, 6H) 2.71 (s, 3H) 2.93 (q, J=7.34 Hz, 2H) 7.57 (d, J=2.20 Hz, 1H) 8.22 (d, J=2.20 Hz, 1H).

### Step 4: Preparation of 2-[[6-(2-bromoacetyl)-5-ethylsulfanyl-3-pyridyl]oxy]-2-methyl-propanenitrile (Compound I26)

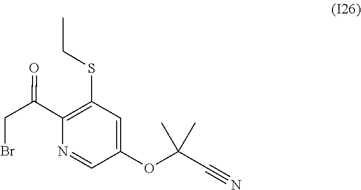 [SMILES: CCSc1cc(OC(C)(C)C#N)cnc1C(=O)CBr]

Trimethyl(phenyl)ammonium tribromide (1.43 g, 3.78 mmol) was added to a 0° C. cooled solution of 2-[(6-acetyl-5-ethylsulfanyl-3-pyridyl)oxy]-2-methyl-propanenitrile (compound I25 prepared as described above) (1.00 g, 3.78 mmol) in tetrahydrofuran (14.4 mL, freshly opened bottle). The resulting orange suspension was stirred at room temperature for 42 hours, before quenching the reaction with water.

The aqueous phase was extracted three times with ethyl acetate, the combined organic phases were washed with brine, dried over sodium sulfate, filtered and concentrated. The crude yellow oil was triturated in cold cyclohexane (15 mL) containing some dichloromethane (1.0 mL) to obtain a precipitate, which was filtered and washed with cyclohexane, yielding the desired compound as a yellow solid (812 mg. The filtrate was purified by flash chromatography over silica gel (ethyl acetate in cyclohexane) to give a second, less pure, portion of desired compound as a yellow oil (500 mg).

¹H NMR (400 MHz, chloroform-d) δ ppm 1.45 (t, J=7.34 Hz, 3H) 1.85 (s, 6H) 2.96 (q, J=7.34 Hz, 2H) 4.82 (s, 2H) 7.59 (d, J=2.57 z, 1H) 8.21 (d, J=2.57 Hz, 1H).

### Step 5: Preparation of 2-[[5-ethylsulfanyl-6-[7-(trifluoromethyl)imidazo[1,2-a]pyridin-2-yl]-3-pyridyl]oxy]-2-methyl-propanenitrile (Compound I28)

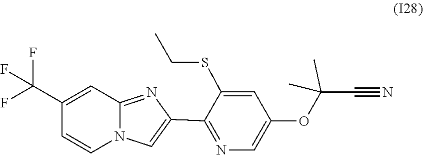 [SMILES: CCSc1cc(OC(C)(C)C#N)cnc1-c1cn2ccc(C(F)(F)F)cc2n1]

A suspension of 2-[[6-(2-bromoacetyl)-5-ethylsulfanyl-3-pyridyl]oxy]-2-methyl-propanenitrile (compound I26 prepared as described above) (100 mg, 0.20 mmol) and 4-(trifluoromethyl)pyridin-2-amine (commercially available) (35 mg, 0.21 mmol) in acetonitrile (1.5 mL) was heated at 70° C. and stirred overnight. Magnesium oxide (8 mg, 0.20 mmol) was added to the reaction mixture and heating was continued for 3 hours to reach completion of the reaction. After cooling down to room temperature, the mixture was poured over water, and the aqueous phase was extracted twice with ethyl acetate. The combined organic phases were washed with brine, dried over sodium sulfate, filtered and concentrated. The crude material was purified partially by flash chromatography over silica gel (ethyl acetate in cyclohexane) to afford the desired product (60 mg) as a yellow oil.

¹H NMR (400 MHz, chloroform-d) δ ppm 1.44 (t, J=7.34 Hz, 3H) 1.81 (s, 6H) 3.04 (q, J=7.34 Hz, 2H) 7.02 (dd, J1=7.34; J2=1.65 Hz, 1H) 7.65 (d, J=2.57 Hz, 1H) 8.06 (s, 1H) 8.29 (d, J=7.34 Hz, 1H) 8.32 (d, J=2.57 Hz, 1H) 8.37 (d, J=1.65 Hz, 1H).

### Step 6: Preparation of 2-[[5-ethylsulfonyl-6-[7-(trifluoromethyl)imidazo[1,2-a]pyridin-2-yl]-3-pyridyl]oxy]-2-methyl-propanenitrile (Compound P8)

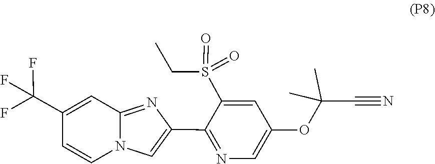 [SMILES: CCS(=O)(=O)c1cc(OC(C)(C)C#N)cnc1-c1cn2ccc(C(F)(F)F)cc2n1]

2-[[5-Ethylsulfanyl-6-[7-(trifluoromethyl)imidazo[1,2-a]pyridin-2-yl]-3-pyridyl]oxy]-2-methyl-propanenitrile (compound I28 prepared as described above) was treated under the same conditions as described in step 4 of EXAMPLE P2 to give the desired compound.

LCMS (method 1): m/z 439 [M+H]⁺; retention time: 0.98 min.

### Example P9: Preparation of 2-[[5-ethylsulfonyl-6-[7-(trifluoromethyl)imidazo[1,2-b]pyridazin-2-yl]-3-pyridyl]oxy]-2-methyl-propanenitrile (Compound P9)

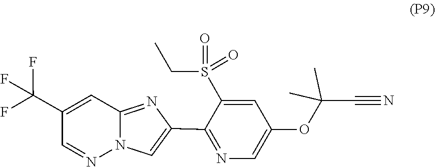 [SMILES: CCS(=O)(=O)c1cc(OC(C)(C)C#N)cnc1-c1cn2ncc(C(F)(F)F)cc2n1]

### Step 1: Preparation of 2-[[5-ethylsulfanyl-6-[7-(trifluoromethyl)imidazo[1,2-b]pyridazin-2-yl]-3-pyridyl]oxy]-2-methyl-propanenitrile (Compound I11)

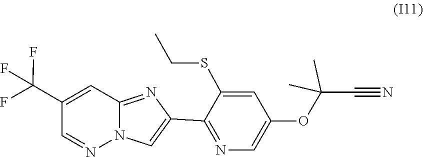 [SMILES: CCSc1cc(OC(C)(C)C#N)cnc1-c1cn2ncc(C(F)(F)F)cc2n1]

2-[[6-(2-Bromoacetyl)-5-ethylsulfanyl-3-pyridyl]oxy]-2-methyl-propanenitrile (compound I26 prepared as described above) and 5-(trifluoromethyl)pyridazin-3-amine (CAS 1211591-88-6) were treated under analogous conditions as described in step 5 of EXAMPLE P8 to give the desired compound.

LCMS (method 1): m/z 408 [M+H]⁺; retention time: 1.09 min.

### Step 2: Preparation of 2-[[5-ethylsulfonyl-6-[7-(trifluoromethyl)imidazo[1,2-b]pyridazin-2-yl]-3-pyridyl]oxy]-2-methyl-propanenitrile (Compound P9)

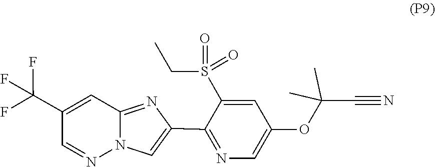 [SMILES: CCS(=O)(=O)c1cc(OC(C)(C)C#N)cnc1-c1cn2ncc(C(F)(F)F)cc2n1]

2-[[5-Ethylsulfanyl-6-[7-(trifluoromethyl)imidazo[1,2-b]pyridazin-2-yl]-3-pyridyl]oxy]-2-methyl-propanenitrile (compound I11 prepared as described above) was treated under the same conditions as described in step 4 of EXAMPLE P2 to give the desired compound.

LCMS (method 1): m/z 440 [M+H]⁺; retention time: 0.99 min.

### Example P14: Preparation of 2-[[5-ethylsulfonyl-6-[1-methyl-5-(trifluoromethylsulfonyl)benzimidazol-2-yl]-3-pyridyl]oxy]-2-methyl-propanenitrile (Compound P14)

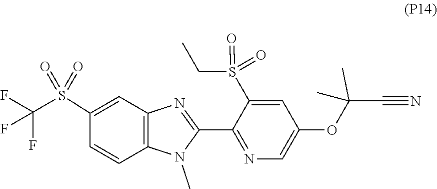 [SMILES: CCS(=O)(=O)c1cc(OC(C)(C)C#N)cnc1-c1nc2cc(S(=O)(=O)C(F)(F)F)ccc2n1C]

### Step 1: Preparation of N-[2-amino-4-(trifluoromethylsulfanyl)phenyl]-5-(1-cyano-1-methyl-ethoxy)-3-ethylsulfanyl-N-methyl-pyridine-2-carboxamide (Compound I41)

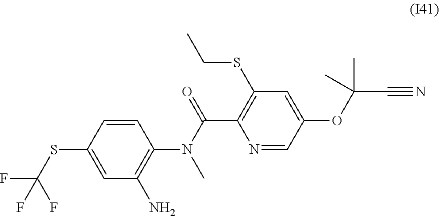 [SMILES: CCSc1cc(OC(C)(C)C#N)cnc1C(=O)N(C)c1ccc(SC(F)(F)F)cc1N]

To a solution of N1-methyl-4-(trifluoromethylsulfanyl)benzene-1,2-diamine (WO2012/086848) (400 mg, 1.80 mmol, 1.05 eq.) and triethylamine (3.0 equiv) in tetrahydrofuran (15 mL) at 0° C. was added a solution of 5-(1-cyano-1-methyl-ethoxy)-3-ethylsulfanyl-pyridine-2-carbonyl chloride (compound I32 prepared as described below) (1.0 eq.) in tetrahydrofuran (15 mL) dropwise. The reaction mixture was stirred at room temperature for 3 hours, then evaporated in vacuo. The residue was diluted with water (50 mL) and extracted with ethyl acetate (3×50 mL). The combined organic layers were washed with brine (20 mL), dried over sodium sulfate, filtered and concentrated under reduced pressure to afford the crude desired product. This material was used as such in the next step.

LCMS (method 3): m/z 471 [M+H]⁺; retention time: 1.64 min.

### Step 2: Preparation of 2-[[5-ethylsulfanyl-6-[1-methyl-5-(trifluoromethylsulfanyl)benzimidazol-2-yl]-3-pyridyl]oxy]-2-methyl-propanenitrile (Compound I40)

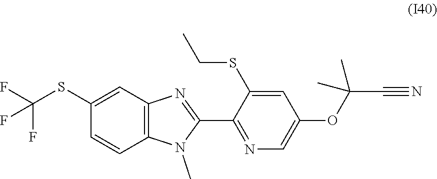 [SMILES: CCSc1cc(OC(C)(C)C#N)cnc1-c1nc2cc(SC(F)(F)F)ccc2n1C]

A solution of N-[2-amino-4-(trifluoromethylsulfanyl)phenyl]-5-(1-cyano-1-methyl-ethoxy)-3-ethylsulfanyl-N-methyl-pyridine-2-carboxamide (compound I41 prepared as described above) (800 mg, 1.70 mmol) in glacial acetic acid (12 mL) was heated at 150° C. for 2 hours. The reaction mixture was concentrated under reduced pressure, the residue quenched with water (50 mL) and extracted with ethyl acetate (3×50 mL). The combined organic layers were washed with brine (20 mL), dried over sodium sulfate, filtered and concentrated in vacuo. The residue was purified by column chromatography (combiflash) on silica gel (30% ethyl acetate-cyclohexane) to afford the desired product as an off-white solid.

LCMS (method 5): m/z 453 [M+H]⁺; retention time: 1.12 min.

### Step 3: Preparation of 2-[[5-ethylsulfonyl-6-[1-methyl-5-(trifluoromethylsulfanyl)benzimidazol-2-yl]-3-pyridyl]oxy]-2-methyl-propanenitrile (Compound P13)

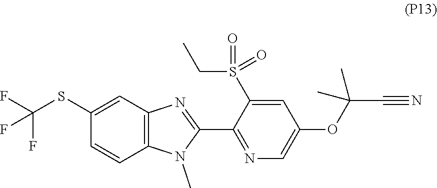 [SMILES: CCS(=O)(=O)c1cc(OC(C)(C)C#N)cnc1-c1nc2cc(SC(F)(F)F)ccc2n1C]

2-[[5-Ethylsulfanyl-6-[1-methyl-5-(trifluoromethylsulfanyl)benzimidazol-2-yl]-3-pyridyl]oxy]-2-methyl-propanenitrile (compound I40 prepared as described above) was treated with 2.2 eq. of oxidant 3-chlorobenzenecarboperoxoic acid under analogous conditions as described in step 4 of EXAMPLE P2 to give the desired compound after stirring for 2 hours at room temperature. The crude product obtained after extractive workup was purified by column chromatography (combiflash) on silica gel (40% ethyl acetate in cyclohexane).

LCMS (method 5): m/z 485 [M+H]⁺; retention time: 1.12 min.

### Step 4: Preparation of 2-[[5-ethylsulfonyl-6-[1-methyl-5-(trifluoromethylsulfonyl)benzimidazol-2-yl]-3-pyridyl]oxy]-2-methyl-propanenitrile (Compound P14)

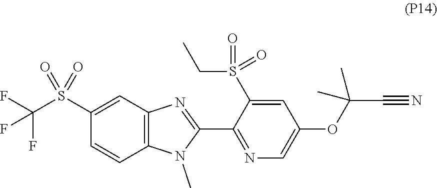 [SMILES: CCS(=O)(=O)c1cc(OC(C)(C)C#N)cnc1-c1nc2cc(S(=O)(=O)C(F)(F)F)ccc2n1C]

2-[[5-Ethylsulfanyl-6-[1-methyl-5-(trifluoromethylsulfanyl)benzimidazol-2-yl]-3-pyridyl]oxy]-2-methyl-propanenitrile (compound I40 prepared as described above) was treated with 4.5 eq. of oxidant 3-chlorobenzenecarboperoxoic acid under analogous conditions as described in step 4 of EXAMPLE P2 to give the desired compound after stirring overnight at room temperature. The crude product obtained after extractive workup was purified by column chromatography (combiflash) on silica gel (40% ethyl acetate in cyclohexane).

LCMS (method 5): m/z 517 [M+H]⁺; retention time: 1.02 min.

### Example P15: Preparation of 2-[[6-(2,2-difluoro-7-methyl-[1,3]dioxolo[4,5-f]benzimidazol-6-yl)-5-ethylsulfonyl-3-pyridyl]oxy]-2-methyl-propanenitrile (Compound P15)

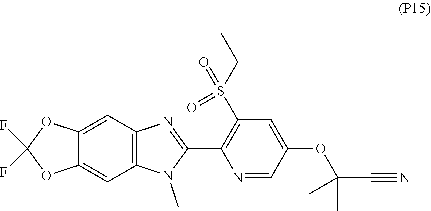 [SMILES: CCS(=O)(=O)c1cc(OC(C)(C)C#N)cnc1-c1nc2cc3c(cc2n1C)OC(F)(F)O3]

### Step 1: Preparation of 5-(1-cyano-1-methyl-ethoxy)-N-[2,2-difluoro-6-(methylamino)-1,3-benzodioxol-5-yl]-3-ethylsulfanyl-pyridine-2-carboxamide (Compound I42)

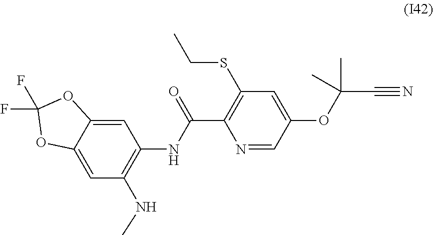 [SMILES: CCSc1cc(OC(C)(C)C#N)cnc1C(=O)Nc1cc2c(cc1NC)OC(F)(F)O2]

To a solution of 5-(1-cyano-1-methyl-ethoxy)-3-ethylsulfanyl-pyridine-2-carboxylic acid (compound I36 prepared as described below) (350 mg, 1.31 mmol)) in ethyl acetate (5.25 mL) under nitrogen at 0° C. were added 2,2-difluoro-N5-methyl-1,3-benzodioxole-5,6-diamine hydrochloric salt (408 mg, 1.71 mmol), N,N-diisopropyl-ethylamine (0.689 mL, 4.02 mmol), and a 50% solution of T3P [propanephosphonic acid anhydride] in methyl-tetrahydrofuran (1.61 mL, 2.63 mmol) dropwise. The mixture was stirred at 0° C. for 2 hours, then diluted with aqueous sodium hydrogen carbonate. The product was extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product.

LCMS (method 1): m/z 451 [M+H]⁺; retention time: 1.17 min.

### Step 2: Preparation of 2-[[6-(2,2-difluoro-7-methyl-[1,3]dioxolo[4,5-f]benzimidazol-6-yl)-5-ethylsulfanyl-3-pyridyl]oxy]-2-methyl-propanenitrile (Compound I43)

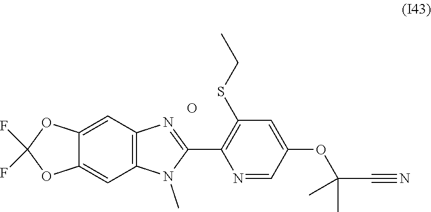

A solution of 5-(1-cyano-1-methyl-ethoxy)-N-[2,2-difluoro-6-(methylamino)-1,3-benzodioxol-5-yl]-3-ethylsulfanyl-pyridine-2-carboxamide (compound I42 prepared as described above) (245 mg, 0.54 mmol) was refluxed in glacial acetic acid (2.2 mL) for one hour. The mixture was concentrated in vacuo and the residue diluted with ethyl acetate and aqueous sodium hydrogen carbonate. The product was extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient ethyl acetate in cyclohexane) to afford the desired product.

LCMS (method 1): m/z 433 [M+H]⁺; retention time: 1.11 min.

### Step 3: Preparation of 2-[[6-(2,2-difluoro-7-methyl-[1,3]dioxolo[4,5-f]benzimidazol-6-yl)-5-ethylsulfonyl-3-pyridyl]oxy]-2-methyl-propanenitrile (Compound P15)

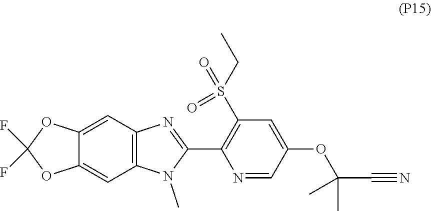 [SMILES: CCS(=O)(=O)c1cc(OC(C)(C)C#N)cnc1-c1nc2cc3c(cc2n1C)OC(F)(F)O3]

2-[[6-(2,2-difluoro-7-methyl-[1,3]dioxolo[4,5-f]benzimidazol-6-yl)-5-ethylsulfanyl-3-pyridyl]oxy]-2-methyl-propanenitrile (compound I43 prepared as described above) in ethyl acetate was treated with 2.3 eq. of oxidant 3-chlorobenzenecarboperoxoic acid under analogous conditions as described in step 4 of EXAMPLE P2 to give the desired compound after stirring for 4 hours at room temperature. The crude product obtained after extractive workup was purified by column chromatography (combiflash) on silica gel (10-45% ethyl acetate in cyclohexane).

LCMS (method 1): m/z 465 [M+H]⁺; retention time: 1.05 min. ¹H NMR (400 MHz, chloroform-d) δ ppm 1.39 (t, 3H), 1.91 (s, 6H), 3.77 (s, 3H), 3.87 (q, 2H), 7.13 (s, 1H), 7.44 (s, 1H), 8.30 (d, 1H), 8.83 (d, 1H).

### Example P16: Preparation of 2-[[6-(2,2-difluoro-[1,3]dioxolo[4,5-f][1,3]benzoxazol-6-yl)-5-ethylsulfonyl-3-pyridyl]oxy]-2-methyl-propanenitrile (Compound P16)

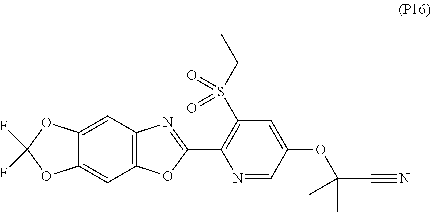 [SMILES: CCS(=O)(=O)c1cc(OC(C)(C)C#N)cnc1-c1nc2cc3c(cc2o1)OC(F)(F)O3]

### Step 1: Preparation of N-(6-bromo-2,2-difluoro-1,3-benzodioxol-5-yl)-5-(1-cyano-1-methyl-ethoxy)-3-ethylsulfanyl-pyridine-2-carboxamide (Compound I44)

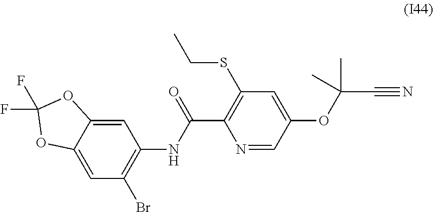 [SMILES: CCSc1cc(OC(C)(C)C#N)cnc1C(=O)Nc1cc2c(cc1Br)OC(F)(F)O2]

To a solution of 5-(1-cyano-1-methyl-ethoxy)-3-ethylsulfanyl-pyridine-2-carboxylic acid (compound I36 prepared as described below) (250 mg, 0.94 mmol)) in ethyl acetate (3.75 mL) under nitrogen at 0° C. were added 6-bromo-2,2-difluoro-1,3-benzodioxol-5-amine (CAS 887267-84-7) (241 mg, 0.94 mmol), triethylamine (0.196 mL, 1.41 mmol), and a 50% solution of T3P [propanephosphonic acid anhydride] in methyl-tetrahydrofuran (0.747 mL, 1.22 mmol) dropwise. The mixture was stirred at room temperature for 16 hours, then diluted with aqueous sodium hydrogen carbonate. The product was extracted twice with ethyl acetate, the combined organic layers washed with an aqueous saturated solution of sodium hydrogen carbonate, dried over magnesium sulfate, filtered and concentrated in vacuo. The residue was purified by Combiflash (gradient t-butyl methyl ether in cyclohexane) to afford the desired product.

LCMS (method 1): m/z 500/502 [M+H]⁺; retention time: 1.34 min.

### Step 2: Preparation of 2-[[6-(2,2-difluoro-[1,3]dioxolo[4,5-f][1,3]benzoxazol-6-yl)-5-ethylsulfanyl-3-pyridyl]oxy]-2-methyl-propanenitrile (Compound I45)

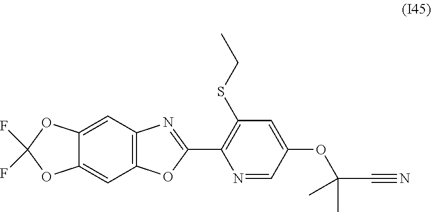 [SMILES: CCSc1cc(OC(C)(C)C#N)cnc1-c1nc2cc3c(cc2o1)OC(F)(F)O3]

A microwave vial was charged with N-(6-bromo-2,2-difluoro-1,3-benzodioxol-5-yl)-5-(1-cyano-1-methyl-ethoxy)-3-ethylsulfanyl-pyridine-2-carboxamide (compound I44 prepared as described above) (131 mg, 0.26 mmol), potassium carbonate (47 mg, 0.34 mmol), copper(I) iodide (10 mg, 0.052 mmol), N,N′-dimethylethylenediamine (5.7 μL, 0.052 mmol) and toluene (1.3 mL). The mixture was flushed with argon, then heated in the microwave at 150° C. for 4 hours. Additional copper(I) iodide (10 mg) was added and heating continued at 150° C. for 3 hours. The reaction mixture was filtrated over Hyflo and the residue washed with ethyl acetate and water. The layers of the filtrate were separated, the aqueous phase extracted twice with ethyl acetate, the combined organic layers washed with brine, dried over magnesium sulfate, filtered and concentrated in vacuo. The residue was purified by Combiflash (gradient ethyl acetate in cyclohexane) to afford the desired product.

LCMS (method 1): m/z 420 [M+H]⁺; retention time: 1.20 min.

### Step 3: Preparation of 2-[[6-(2,2-difluoro-[1,3]dioxolo[4,5-f][1,3]benzoxazol-6-yl)-5-ethylsulfonyl-3-pyridyl]oxy]-2-methyl-propanenitrile (Compound P16)

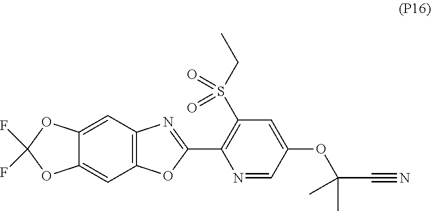 [SMILES: CCS(=O)(=O)c1cc(OC(C)(C)C#N)cnc1-c1nc2cc3c(cc2o1)OC(F)(F)O3]

2-[[6-(2,2-difluoro-[1,3]dioxolo[4,5-f][1,3]benzoxazol-6-yl)-5-ethylsulfanyl-3-pyridyl]oxy]-2-methyl-propanenitrile (compound I45 prepared as described above) in ethyl acetate was treated with 2.2 eq. of oxidant 3-chlorobenzenecarboperoxoic acid under analogous conditions as described in step 4 of EXAMPLE P2 to give the desired compound after stirring for 16 hours at room temperature. The crude product obtained after extractive workup was purified by column chromatography (combiflash) on silica gel (0-45% ethyl acetate in cyclohexane).

LCMS (method 1): m/z 452 [M+H]⁺; retention time: 1.09 mi. ¹H NMR (400 MHz, chloroform-d) δ ppm 1.46 (t, 3H), 1.91 (s, 6H), 4.05 (q, 2H), 7.43 (1, 1H), 7.51 (s, 1H), 8.37 (d, 1H), 8.88 (d, 1H).

TABLE P
Examples of compounds of formula (I)

| | | | LCMS | | | |
|---|---|---|---|---|---|---|
| | | | | [M + H]⁺ | | |
| | | | | (meas- | | Mp |
| No. | IUPAC name | Structures | Rₜ (min) | ured) | Method | (° C.) |
| P1 | 2-[[5-(cyclopropylmethyl- sulfonyl)-6-[7-methyl-3- (trifluoromethyl)imidazo[4,5- c]pyridazin-6-yl]-3-pyridyl] oxy]-2-methyl-propanenitrile | 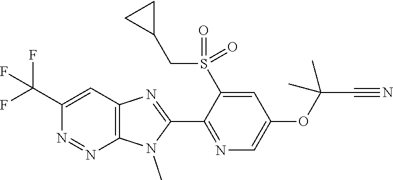 [SMILES: Cn1c(-c2ncc(OC(C)(C)C#N)cc2S(=O)(=O)CC2CC2)nc2cc(C(F)(F)F)nnc21] | 1.17 | 481 | 5 | 142- 144 |
| P2 | 2-[[5-ethylsulfonyl-6-[7- methyl-3-(trifluoromethyl) imidazo[4,5-c]pyridazin-6- yl]-3-pyridyl]oxy]-2- methyl-propanenitrile | 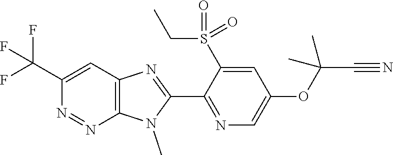 [SMILES: CCS(=O)(=O)c1cc(OC(C)(C)C#N)cnc1-c1nc2cc(C(F)(F)F)nnc2n1C] | 0.98 | 455 | 1 | 139- 140 |
| P3 | 2-[[5-ethylsulfonyl-6-[3- methyl-6-(trifluoromethyl) imidazo[4,5-b]pyridin-2-yl]- 3-pyridyl]oxy]-2-methyl- propanenitrile | 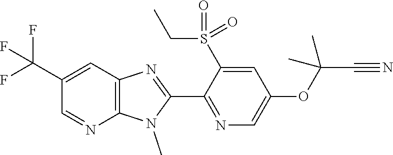 [SMILES: CCS(=O)(=O)c1cc(OC(C)(C)C#N)cnc1-c1nc2cc(C(F)(F)F)cnc2n1C] | 1.05 | 454 | 1 | 139- 140 |
| P4 | 2-[[5-ethylsulfonyl-2-methyl- 6-[3-methyl-6-(trifluoromethyl) imidazo[4,5-b]pyridin-2-yl]-3- pyridyl]oxy]-2-methyl- propanenitrile | 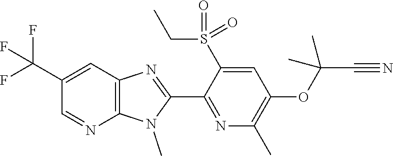 [SMILES: CCS(=O)(=O)c1cc(OC(C)(C)C#N)c(C)nc1-c1nc2cc(C(F)(F)F)cnc2n1C] | 1.07 | 468 | 1 | 175- 177 |
| P5 | 2-[[5-ethylsulfonyl-6-[5- methoxy-3-methyl-4-oxo- 6-(trifluoromethyl)imidazo [4,5-c]pyridin-2-yl]-3- pyridyl]oxy]-2-methyl- propanenitrile | 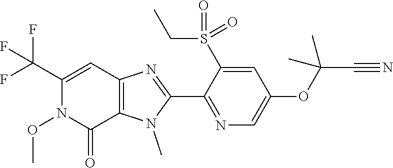 [SMILES: CCS(=O)(=O)c1cc(OC(C)(C)C#N)cnc1-c1nc2cc(C(F)(F)F)n(OC)c(=O)c2n1C] | 1.02 | 500 | 5 | 188- 190 |
| P6 | 2-[[6-[5-ethyl-3-methyl-4- oxo-6-(trifluoromethyl) imidazo[4,5-c]pyridin-2-yl]- 5-ethylsulfonyl-3- pyridyl]oxy]-2-methyl- propanenitrile | 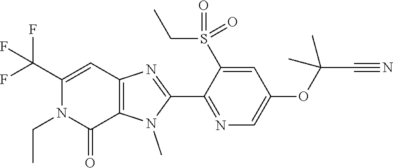 [SMILES: CCn1c(C(F)(F)F)cc2nc(-c3ncc(OC(C)(C)C#N)cc3S(=O)(=O)CC)n(C)c2c1=O] | 1.05 | 498 | 5 | 162- 164 |
| P7 | 2-[[6-[5-cyclopropyl-3- methyl-4-oxo-6-(trifluoro- methyl)imidazo[4,5- c]pyridin-2-yl]-5-ethyl- sulfonyl-3-pyridyl]oxy]- 2-methyl-propanenitrile | 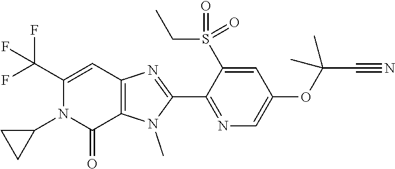 [SMILES: CCS(=O)(=O)c1cc(OC(C)(C)C#N)cnc1-c1nc2cc(C(F)(F)F)n(C3CC3)c(=O)c2n1C] | 1.46 | 510 | 3 | 216- 218 |
| P8 | 2-[[5-ethylsulfonyl-6-[7- (trifluoromethyl)imidazo[1,2- a]pyridin-2-yl]-3-pyridyl] oxy]-2-methyl-propanenitrile | 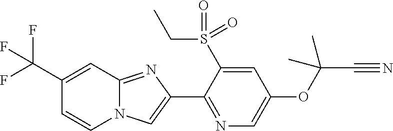 [SMILES: CCS(=O)(=O)c1cc(OC(C)(C)C#N)cnc1-c1cn2ccc(C(F)(F)F)cc2n1] | 0.98 | 439 | 1 | 108- 110 |
| P9 | 2-[[5-ethylsulfonyl-6-[7- (trifluoromethyl)imidazo [1,2-b]pyridazin-2-yl]-3- pyridyl]oxy]-2-methyl- propanenitrile | 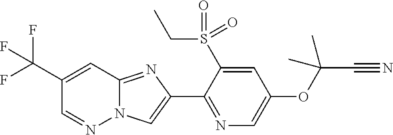 [SMILES: CCS(=O)(=O)c1cc(OC(C)(C)C#N)cnc1-c1cn2ncc(C(F)(F)F)cc2n1] | 0.99 | 440 | 1 | 165- 166 |
| P10 | 2-[3-ethylsulfonyl-4-[6- (trifluoromethyl)pyrazolo [4,3-c]pyridin-2-yl]phenoxy]- 2-methyl-propanenitrile | 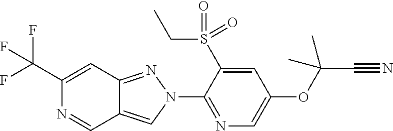 [SMILES: CCS(=O)(=O)c1cc(OC(C)(C)C#N)cnc1-n1cc2cnc(C(F)(F)F)cc2n1] | 1.02 | 439 | 5 | 172- 174 |
| P11 | 2-[[5-ethylsulfonyl-2- methyl-6-[7-methyl-3- (trifluoromethyl)imidazo [4,5-c]pyridazin-6-yl]-3- pyridyl]oxy]-2-methyl- propanenitrile | 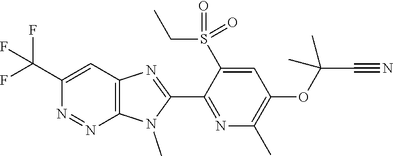 [SMILES: CCS(=O)(=O)c1cc(OC(C)(C)C#N)c(C)nc1-c1nc2cc(C(F)(F)F)nnc2n1C] | 1.00 | 469 | 1 | 151- 153 |
| P12 | 2-[3-ethylsulfonyl-4-[7- methyl-3-(trifluoromethyl) imidazo[4,5-c]pyridazin-6- yl]phenoxy]-2-methyl- propanenitrile | 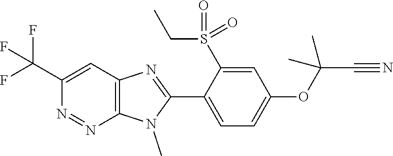 [SMILES: CCS(=O)(=O)c1cc(OC(C)(C)C#N)ccc1-c1nc2cc(C(F)(F)F)nnc2n1C] | 0.98 | 454 | 4 | 196- 198 |
| P13 | 2-[[5-ethylsulfonyl-6-[1- methyl-5-(trifluoromethyl- sulfanyl)benzimidazol-2- yl]-3-pyridyl]oxy]-2- methyl-propanenitrile | 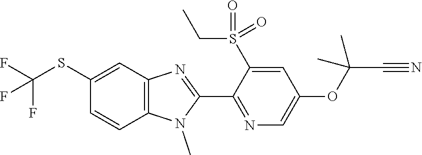 [SMILES: CCS(=O)(=O)c1cc(OC(C)(C)C#N)cnc1-c1nc2cc(SC(F)(F)F)ccc2n1C] | 1.12 | 485 | 5 | 123- 125 |
| P14 | 2-[[5-ethylsulfonyl-6-[1- methyl-5-(trifluoromethyl- sulfonyl)benzimidazol-2- yl]-3-pyridyl]oxy]-2-methyl- propanenitrile | 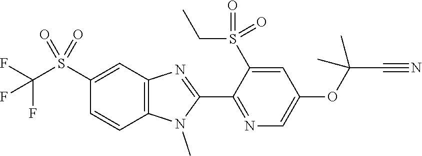 [SMILES: CCS(=O)(=O)c1cc(OC(C)(C)C#N)cnc1-c1nc2cc(S(=O)(=O)C(F)(F)F)ccc2n1C] | 1.02 | 517 | 5 | 168- 170 |
| P15 | 2-[[6-(2,2-difluoro-7-methyl- [1,3]dioxolo[4,5-f]benz- imidazol-6-yl)-5-ethyl- sulfonyl- 3-pyridyl]oxy]-2- methyl-propanenitrile | 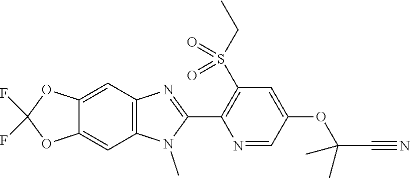 [SMILES: CCS(=O)(=O)c1cc(OC(C)(C)C#N)cnc1-c1nc2cc3c(cc2n1C)OC(F)(F)O3] | 1.05 | 465 | 1 | 155- 158 |
| P16 | 2-[[6-(2,2-difluoro-[1,3] dioxolo[4,5-f][1,3]benz- oxazol-6-yl)-5-ethylsulfonyl- 3-pyridyl]oxy]-2- methyl-propanenitrile | 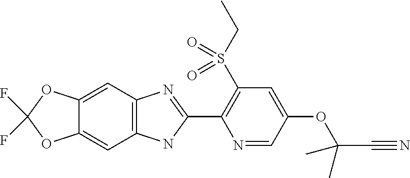 [SMILES: CCS(=O)(=O)c1cc(OC(C)(C)C#N)cnc1-c1nc2cc3c(cc2[nH]1)OC(F)(F)O3] | 1.09 | 452 | 1 | — |

TABLE I
Examples of intermediate compounds of formula (II), (III), (V), (Va), (Vb), (Vc), (VII), (XXII),
(XXIII), (XXIV), (XXV-c), (XXV-a), (XXVI), (XXVIII), (XXIX-a) and (XXIX-c)

| | | | LCMS | | | | |
|---|---|---|---|---|---|---|---|
| | | | | [M + H]⁺ | Meth- | Mp | ¹H |
| No. | IUPAC name | Structures | Rₜ (min) | (measured) | od | (° C.) | NMR |
| I1 | 5-ethylsulfanyl-6-[7- methyl-3-(trifluoro- methyl)imidazo[4,5-c] pyridazin-6- yl]pyridin-3-ol | 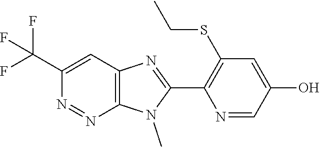 [SMILES: CCSc1cc(O)cnc1-c1nc2cc(C(F)(F)F)nnc2n1C] | 0.86 | 356 | 1 | 216- 218 | 1) |
| I2 | 2-[[5-ethylsulfanyl-6- [7-methyl-3- (trifluoromethyl) imidazo[4,5-c]pyridazin- 6-yl]-3-pyridyl]oxy]-2- methyl-propanamide | 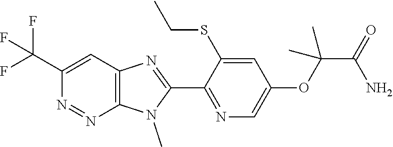 [SMILES: CCSc1cc(OC(C)(C)C(N)=O)cnc1-c1nc2cc(C(F)(F)F)nnc2n1C] | | | | 163- 165 | |
| I3 | 2-[[5-ethylsulfanyl-6- [7-methyl-3- (trifluoromethyl) imidazo[4,5-c]pyridazin- 6-yl]-3-pyridyl]oxy]-2- methyl-propanenitrile | 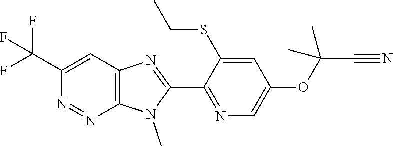 [SMILES: CCSc1cc(OC(C)(C)C#N)cnc1-c1nc2cc(C(F)(F)F)nnc2n1C] | 1.04 | 423 | 1 | | 2) |
| I4 | 5-ethylsulfanyl-6-[3- methyl-6- (trifluoromethyl) imidazo[4,5-b]pyridin- 2-yl]pyridin-3-ol | 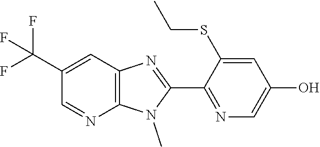 [SMILES: CCSc1cc(O)cnc1-c1nc2cc(C(F)(F)F)cnc2n1C] | 0.94 | 355 | 1 | 194- 195 | |
| I5 | 2-[[5-ethylsulfanyl-6- [3-methyl-6- (trifluoromethyl) imidazo[4,5-b]pyridin- 2-yl]-3-pyridyl] oxy]acetonitrile | 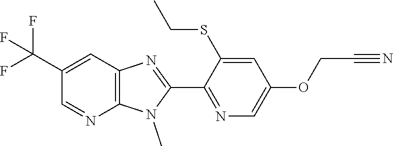 [SMILES: CCSc1cc(OCC#N)cnc1-c1nc2cc(C(F)(F)F)cnc2n1C] | 1.01 | 394 | 1 | | |
| I6 | 2-[[5-ethylsulfanyl-6- [3-methyl-6-(trifluoro- methyl)imidazo[4,5-b] pyridin-2-yl]- 3-pyridyl]oxy]-2- methyl-propanenitrile | 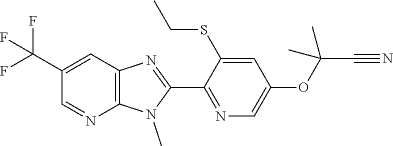 [SMILES: CCSc1cc(OC(C)(C)C#N)cnc1-c1nc2cc(C(F)(F)F)cnc2n1C] | 1.11 | 422 | 1 | | |
| I7 | 5-(cyclopropyl- methylsulfanyl)-6-[7- methyl-3- (trifluoromethyl) imidazo[4,5-c]pyridazin- 6-yl]pyridin-3-ol | 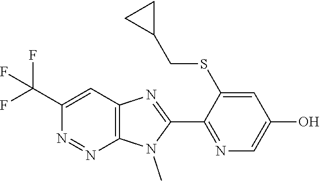 [SMILES: Cn1c(-c2ncc(O)cc2SCC2CC2)nc2cc(C(F)(F)F)nnc21] | 1.40 | 382 | 3 | | |
| I8 | 2-[[5-(cyclopropyl- methylsulfanyl)-6-[7- methyl-3- (trifluoromethyl) imidazo[4,5-c]pyridazin- 6-yl]-3-pyridyl]oxy]-2- methyl-propanamide | 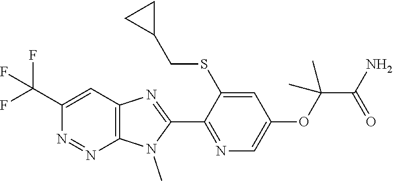 [SMILES: Cn1c(-c2ncc(OC(C)(C)C(N)=O)cc2SCC2CC2)nc2cc(C(F)(F)F)nnc21] | 1.13 | 467 | 5 | | |
| I9 | 2-[[5-(cyclopropyl- methylsulfanyl)-6-[7- methyl-3- (trifluoromethyl) imidazo[4,5-c]pyridazin- 6-yl]-3-pyridyl]oxy]-2- methyl-propanenitrile | 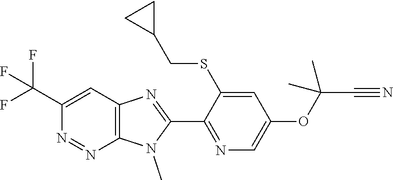 [SMILES: Cn1c(-c2ncc(OC(C)(C)C#N)cc2SCC2CC2)nc2cc(C(F)(F)F)nnc21] | 1.21 | 449 | 5 | | |
| I10 | 2-[[5-ethylsulfanyl-6- [5-methoxy-3- methyl-4-oxo-6- (trifluoromethyl) imidazo[4,5-c]pyridin- 2-yl]-3-pyridyl]oxy]-2- methyl-propanenitrile | 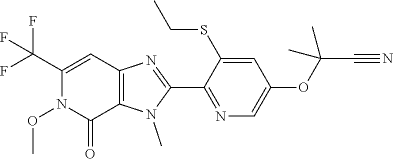 [SMILES: CCSc1cc(OC(C)(C)C#N)cnc1-c1nc2cc(C(F)(F)F)n(OC)c(=O)c2n1C] | 1.07 | 468 | 4 | | |
| I11 | 2-[[5-ethylsulfanyl-6- [7-(trifluoromethyl) imidazo[1,2-b]pyridazin- 2-yl]-3-pyridyl]oxy]-2- methyl-propanenitrile | 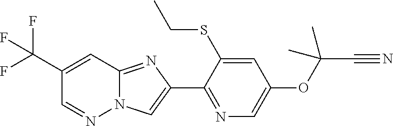 [SMILES: CCSc1cc(OC(C)(C)C#N)cnc1-c1cn2ncc(C(F)(F)F)cc2n1] | 1.09 | 408 | 1 | | |
| I12 | 3-ethylsulfanyl-4-[7- methyl-3- (trifluoromethyl) imidazo[4,5-c]pyridazin- 6-yl]phenol | 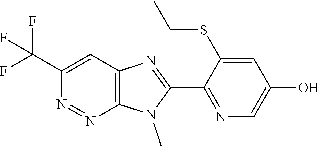 [SMILES: CCSc1cc(O)cnc1-c1nc2cc(C(F)(F)F)nnc2n1C] | 1.34 | 355 | 3 | | |
| I13 | 5-ethylsulfanyl-2- iodo-6-[3-methyl-6- (trifluoromethyl) imidazo[4,5-b]pyridin- 2-yl]pyridin-3-ol | 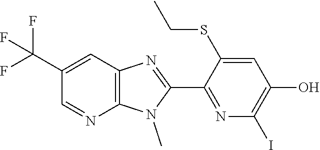 [SMILES: CCSc1cc(O)c(I)nc1-c1nc2cc(C(F)(F)F)cnc2n1C] | 1.06 | 480 | 1 | | |
| I14 | 5-ethylsulfanyl-2- methyl-6-[3-methyl-6- (trifluoromethyl) imidazo[4,5-b]pyridin- 2-yl]pyridin-3-ol | 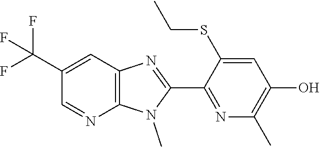 [SMILES: CCSc1cc(O)c(C)nc1-c1nc2cc(C(F)(F)F)cnc2n1C] | 0.96 | 369 | 1 | | |
| I15 | 2-[[5-ethylsulfanyl-2- methyl-6-[3-methyl-6- (trifluoromethyl) imidazo[4,5-b]pyridin- 2-yl]-3-pyridyl]oxy] acetonitrile | 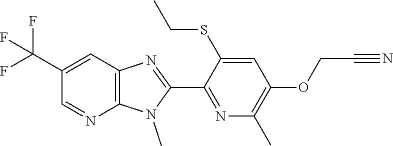 [SMILES: CCSc1cc(OCC#N)c(C)nc1-c1nc2cc(C(F)(F)F)cnc2n1C] | 1.05 | 408 | 1 | | |
| I16 | 2-[[5-ethylsulfanyl-2- methyl-6-[3-methyl-6- (trifluoromethyl) imidazo[4,5-b]pyridin- 2-yl]-3-pyridyl]oxy]-2- methyl-propanenitrile | 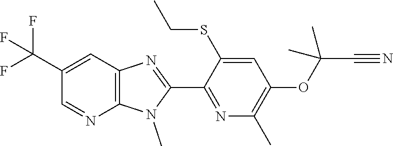 [SMILES: CCSc1cc(OC(C)(C)C#N)c(C)nc1-c1nc2cc(C(F)(F)F)cnc2n1C] | 1.16 | 436 | 1 | | |
| I17 | 5-cyclopropyl-2-(3- ethylsulfanyl-5- hydroxy-2-pyridyl)-3- methyl-6- (trifluoromethyl) imidazo[4,5-c]pyridin- 4-one | 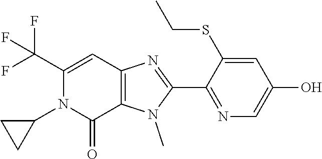 [SMILES: CCSc1cc(O)cnc1-c1nc2cc(C(F)(F)F)n(C3CC3)c(=O)c2n1C] | 1.36 | 411 | 3 | | 3) |
| I18 | 2-[[6-[5-cyclopropyl- 3-methyl-4-oxo-6- (trifluoromethyl) imidazo[4,5-c]pyridin- 2-yl]-5-ethylsulfanyl-3- pyridyl]oxy]acetonitrile | 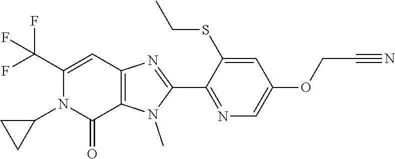 [SMILES: CCSc1cc(OCC#N)cnc1-c1nc2cc(C(F)(F)F)n(C3CC3)c(=O)c2n1C] | 1.48 | 450 | 3 | | |
| I19 | 2-[[6-[5-cyclopropyl- 3-methyl-4-oxo-6- (trifluoromethyl) imidazo[4,5-c]pyridin- 2-yl]-5-ethylsulfanyl-3- pyridyl]oxy]-2- methyl-propanenitrile | 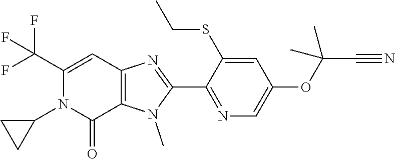 [SMILES: CCSc1cc(OC(C)(C)C#N)cnc1-c1nc2cc(C(F)(F)F)n(C3CC3)c(=O)c2n1C] | 1.54 | 478 | 3 | | |
| I20 | 5-ethyl-2-(3- ethylsulfanyl-5- hydroxy-2-pyridyl)-3- methyl-6- (trifluoromethyl) imidazo[4,5-c]pyridin- 4-one | 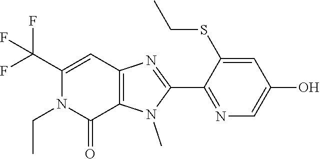 [SMILES: CCSc1cc(O)cnc1-c1nc2cc(C(F)(F)F)n(CC)c(=O)c2n1C] | 1.38 | 399 | 3 | | |
| I21 | 2-[[6-[5-ethyl-3- methyl-4-oxo-6- (trifluoromethyl) imidazo[4,5-c]pyridin- 2-yl]-5-ethylsulfanyl-3- pyridyl]oxy]acetonitrile | 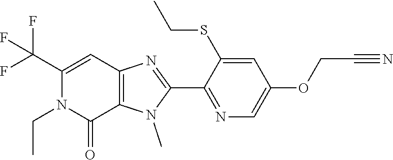 [SMILES: CCSc1cc(OCC#N)cnc1-c1nc2cc(C(F)(F)F)n(CC)c(=O)c2n1C] | 1.01 | 438 | 4 | | |
| I22 | 2-[[6-[5-ethyl-3- methyl-4-oxo-6- (trifluoromethyl) imidazo[4,5-c]pyridin- 2-yl]-5-ethylsulfanyl-3- pyridyl]oxy]-2-methyl- propanenitrile | 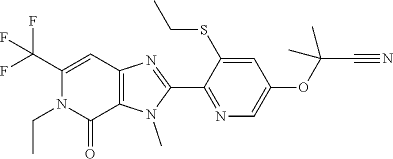 [SMILES: CCSc1cc(OC(C)(C)C#N)cnc1-c1nc2cc(C(F)(F)F)n(CC)c(=O)c2n1C] | 1.10 | 466 | 4 | | |
| I23 | 1-(3-ethylsulfanyl-5- hydroxy-2- pyridyl)ethanone | 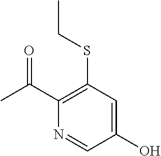 | | | | | 4) |
| I24 | 2-[(6-acetyl-5- ethylsulfanyl-3- pyridyl)oxy]-2- methyl-propanamide | 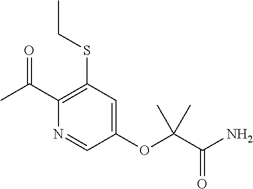 [SMILES: CCSc1cc(OC(C)(C)C(N)=O)cnc1C(C)=O] | | | | | 5) |
| I25 | 2-[(6-acetyl-5- ethylsulfanyl-3- pyridyl)oxy]-2- methyl-propanenitrile | 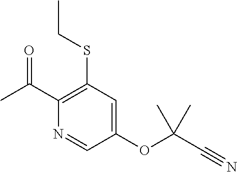 [SMILES: CCSc1cc(OC(C)(C)C#N)cnc1C(C)=O] | | | | | 6) |
| I26 | 2-[[6-(2- bromoacetyl)-5- ethylsulfanyl-3- pyridyl]oxy]-2- methyl-propanenitrile | 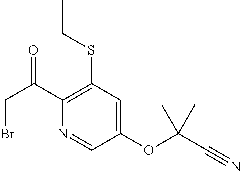 [SMILES: CCSc1cc(OC(C)(C)C#N)cnc1C(=O)CBr] | | | | | 7) |
| I27 | 2-[3-ethylsulfanyl-4- [7-methyl-3- (trifluoromethyl) imidazo[4,5-c]pyridazin- 6-yl]phenoxy]-2- methyl-propanamide | 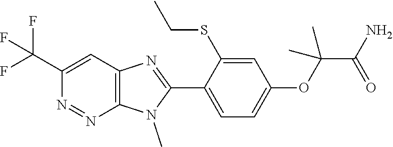 [SMILES: CCSc1cc(OC(C)(C)C(N)=O)ccc1-c1nc2cc(C(F)(F)F)nnc2n1C] | 0.95 | 440 | 4 | 142- 144 | |
| I28 | 2-[[5-ethylsulfanyl-6- [7-(trifluoromethyl) imidazo[1,2-a]pyridin-2- yl]-3-pyridyl]oxy]-2- methyl-propanenitrile | 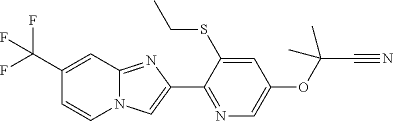 [SMILES: CCSc1cc(OC(C)(C)C#N)cnc1-c1cn2ccc(C(F)(F)F)cc2n1] | 1.02 | 407 | 1 | | 8) |
| I29 | 2-[3-ethylsulfanyl-4- [7-methyl-3- (trifluoromethyl) imidazo[4,5-c]pyridazin- 6-yl]phenoxy]-2- methyl-propanenitrile | 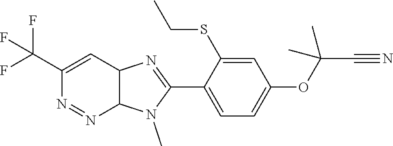 [SMILES: CCSc1cc(OC(C)(C)C#N)ccc1C1=NC2C=C(C(F)(F)F)N=NC2N1C] | 1.07 | 422 | 4 | 126- 128 | |
| I30 | 5-ethylsulfanyl-2- iodo-6-[7-methyl-3- (trifluoromethyl) imidazo[4,5-c]pyridazin- 6-yl]pyridin-3-ol | 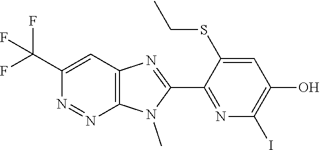 [SMILES: CCSc1cc(O)c(I)nc1-c1nc2cc(C(F)(F)F)nnc2n1C] | 0.98 | 482 | 1 | | |
| I31 | 2-[[5-ethylsulfanyl-6- [3-methyl-6- (trifluoromethyl) imidazo[4,5-b]pyridin-2- yl]-3-pyridyl]oxy]-2- methyl-propanamide | 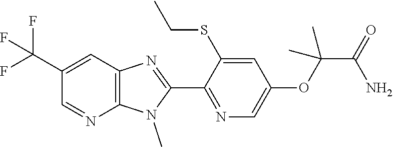 [SMILES: CCSc1cc(OC(C)(C)C(N)=O)cnc1-c1nc2cc(C(F)(F)F)cnc2n1C] | 0.96 | 440 | 1 | 228- 230 | |
| I32 | 5-(1-cyano-1-methyl- ethoxy)-3- ethylsulfanyl- pyridine-2-carbonyl chloride | 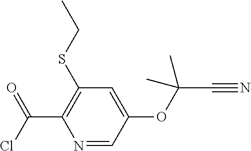 [SMILES: CCSc1cc(OC(C)(C)C#N)cnc1C(=O)Cl] | 0.83 | 294 | 1 | | |
| I33 | methyl 3- ethylsulfanyl-5- hydroxy-pyridine-2- carboxylate | 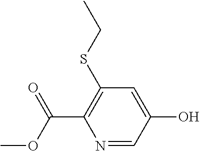 | 0.68 | 214 | 1 | | |
| I34 | methyl 5-(2-amino- 1,1-dimethyl-2-oxo- ethoxy)-3- ethylsulfanyl- pyridine-2- carboxylate | 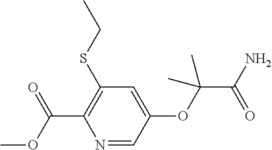 [SMILES: CCSc1cc(OC(C)(C)C(N)=O)cnc1C(=O)OC] | 0.71 | 299 | 1 | | |
| I35 | methyl 5-(1-cyano-1- methyl-ethoxy)-3- ethylsulfanyl- pyridine-2- carboxylate | 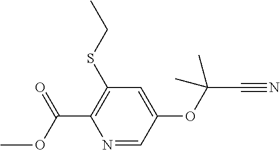 [SMILES: CCSc1cc(OC(C)(C)C#N)cnc1C(=O)OC] | 0.90 | 281 | 1 | | |
| I36 | 5-(1-cyano-1-methyl- ethoxy)-3- ethylsulfanyl- pyridine-2-carboxylic acid | 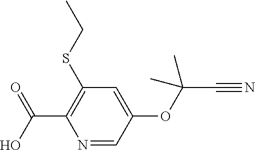 [SMILES: CCSc1cc(OC(C)(C)C#N)cnc1C(=O)O] | 0.82 | 267 | 4 | | |
| I37 | 5-ethylsulfanyl-2- methyl-6-[7-methyl- 3-(tri fluoromethyl) imidazo[4,5- c]pyridazin-6- yl]pyridin-3-ol | 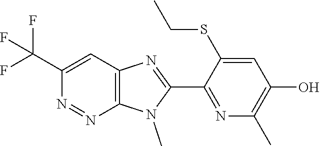 [SMILES: CCSc1cc(O)c(C)nc1-c1nc2cc(C(F)(F)F)nnc2n1C] | 0.91 | 370 | 1 | | |
| I38 | 2-[[5-ethylsulfanyl-2- methyl-6-[7-methyl- 3-(trifluoromethyl) imidazo[4,5- c]pyridazin-6-yl]-3- pyridyl]oxy]-2- methyl-propanamide | 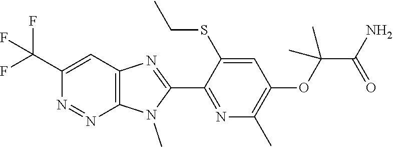 [SMILES: CCSc1cc(OC(C)(C)C(N)=O)c(C)nc1-c1nc2cc(C(F)(F)F)nnc2n1C] | 0.92 | 455 | 1 | | |
| I39 | 2-[[5-ethylsulfanyl-2- methyl-6-[7-methyl- 3-(trifluoromethyl) imidazo[4,5- c]pyridazin-6-yl]-3- pyridyl]oxy]-2- methyl-propanenitrile | 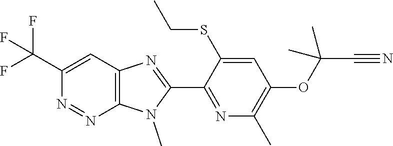 [SMILES: CCSc1cc(OC(C)(C)C#N)c(C)nc1-c1nc2cc(C(F)(F)F)nnc2n1C] | 1.09 | 437 | 1 | | |
| I40 | 2-[[5-ethylsulfanyl-6- [1-methyl-5-(trifluoro- methylsulfanyl) benzimidazol-2- yl]-3-pyridyl]oxy]-2- methyl-propanenitrile | 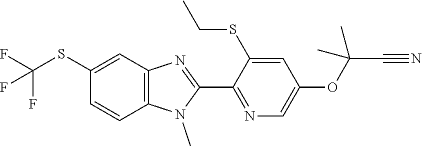 [SMILES: CCSc1cc(OC(C)(C)C#N)cnc1-c1nc2cc(SC(F)(F)F)ccc2n1C] | 1.12 | 453 | 5 | | |
| I41 | N-[2-amino-4- (trifluoromethyl- sulfanyl)phenyl]-5-(1- cyano-1-methyl- ethoxy)-3- ethylsulfanyl-N- methyl-pyridine-2- carboxamide | 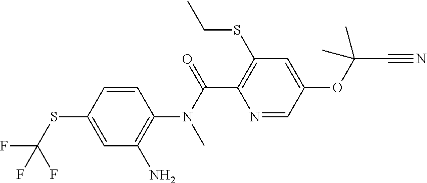 [SMILES: CCSc1cc(OC(C)(C)C#N)cnc1C(=O)N(C)c1ccc(SC(F)(F)F)cc1N] | 1.64 | 471 | 3 | | |
| I42 | 5-(1-cyano-1-methyl- ethoxy)-N-[2,2- difluoro-6- (methylamino)-1,3- benzodioxol-5-yl]-3- ethylsulfanyl- pyridine-2- carboxamide | 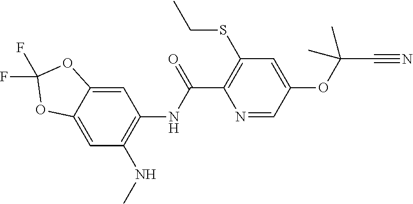 [SMILES: CCSc1cc(OC(C)(C)C#N)cnc1C(=O)Nc1cc2c(cc1NC)OC(F)(F)O2] | 1.17 | 451 | 1 | | |
| I43 | 2-[[6-(2,2-difluoro-7- methyl- [1,3]dioxolo[4,5- f]benzimidazol-6-yl)- 5-ethylsulfanyl-3- pyridyl]oxy]-2- methyl-propanenitrile | 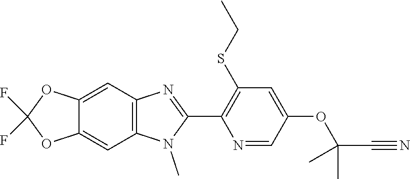 [SMILES: CCSc1cc(OC(C)(C)C#N)cnc1-c1nc2cc3c(cc2n1C)OC(F)(F)O3] | 1.11 | 433 | 1 | | |
| I44 | N-(6-bromo-2,2- difluoro-1,3- benzodioxol-5-yl)-5- (1-cyano-1-methyl- ethoxy)-3- ethylsulfanyl- pyridine-2- carboxamide | 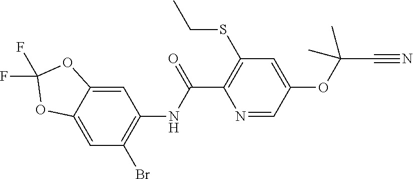 [SMILES: CCSc1cc(OC(C)(C)C#N)cnc1C(=O)Nc1cc2c(cc1Br)OC(F)(F)O2] | 1.34 | 500/502 | 1 | | |
| I45 | 2-[[6-(2,2-difluoro- [1,3]dioxolo[4,5- f][1,3]benzoxazol-6- yl)-5-ethylsulfanyl-3- pyridyl]oxy]-2- methyl-propanenitrile | 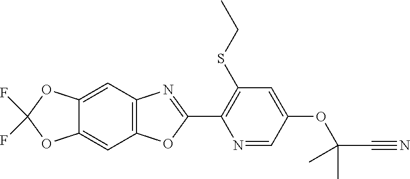 [SMILES: CCSc1cc(OC(C)(C)C#N)cnc1-c1nc2cc3c(cc2o1)OC(F)(F)O3] | 1.20 | 420 | 1 | | |

1) ¹H NMR (400 MHz, dimethylsulfoxide-d6) δ ppm 1.25 (t, J = 7.34 Hz, 3H) 2.99 (q, J = 7.34 Hz, 2H) 4.13 (s, 3H) 7.38 (d, J = 2.20 Hz, 1H) 8.17 (d, J = 2.20 Hz, 1H) 8.55 (s, 1H) 10.94 (s, 1H)
2) ¹H NMR (400 MHz, chloroform-d) δ ppm 1.42 (t, J = 7.34 Hz, 3H) 1.88 (s, 6H) 3.03 (q, J = 7.34 Hz, 2H) 4.31 (s, 3H) 7.72 (d, J = 2.57 Hz, 1H) 8.26 (s, 1H) 8.39 (d, J = 2.57 Hz, 1H)
3) ¹H NMR (400 MHz, chloroform-d) δ ppm 1.06 (brs, 2H) 1.18-1.37 (m, 5H) 2.75 (q, J = 7.38 Hz, 2H) 3.07-3.16 (m, 1H) 4.04 (s, 3H) 7.06 (d, J = 2.45 Hz, 1H) 7.28 (m, 1H) 7.98 (d, J = 2.45 Hz, 1H)
4) ¹H NMR (400 MHz, dimethylsulfoxide-d6) δ ppm 1.28 (t, J = 7.34 Hz, 3H) 2.86 (q, J = 7.34 Hz, 2H) 3.33 (s, 3H) 7.15 (d, J = 2.20 Hz, 1H) 7.98 (d, J = 2.20 Hz, 1H) 10.94 (s br, 1H)
5) ¹H NMR (400 MHz, dimethylsulfoxide-d6) δ ppm 1.28 (t, J = 7.34 Hz, 3H) 1.56 (s, 6H) 1.85 (s, 3H) 2.83 (q, J = 7.34 Hz, 2H) 7.15 (d, J = 2.20 Hz, 1H) 7.33 (s, 1H) 7.45 (s, 1H) 8.04 (d, J = 2.20 Hz, 1H)
6) ¹H NMR (400 MHz, chloroform-d) δ ppm 1.44 (t, J = 7.34 Hz, 3H) 1.83 (s, 6H) 2.71 (s, 3H) 2.93 (q, J = 7.34 Hz, 2H) 7.57 (d, J = 2.20 Hz, 1H) 8.22 (d, J = 2.20 Hz, 1H)
7) ¹H NMR (400 MHz, chloroform-d) δ ppm 1.45 (t, J = 7.34 Hz, 3H) 1.85 (s, 6H) 2.96 (q, J = 7.34 Hz, 2H) 4.82 (s, 2H) 7.59 (d, J = 2.57z, 1H) 8.21 (d, J = 2.57 Hz, 1H)
8) ¹H NMR (400 MHz, chloroform-d) δ ppm 1.44 (t, J = 7.34 Hz, 3H) 1.81 (s, 6H) 3.04 (q, J = 7.34 Hz, 2H) 7.02 (dd, J1= 7.34;J2= 1 .65 Hz, 1H) 7.65 (d, J = 2.57 Hz, 1H) 8.06 (s, 1H) 8.29 (d, J = 7.34 Hz, 1H) 8.32 (d J = 2.57 Hz, 1H) 8.37 (d, J = 1.65 Hz, 1H)

### Example I32: Preparation of 5-(1-cyano-1-methyl-ethoxy)-3-ethylsulfanyl-pyridine-2-carbonyl chloride (Compound I32)

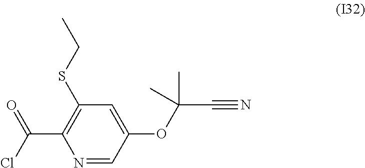 [SMILES: CCSc1cc(OC(C)(C)C#N)cnc1C(=O)Cl]

### Step 1: Preparation of methyl 3-ethylsulfanyl-5-hydroxy-pyridine-2-carboxylate (Compound I33)

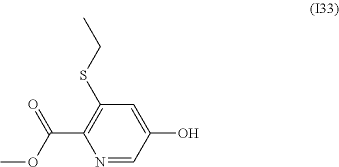

To a solution of methyl 5-bromo-3-ethylsulfanyl-pyridine-2-carboxylate (prepared as described in WO 2016/026848) (10.0 g, 36.21 mmol) in acetonitrile (72 ml) were added cesium carbonate (25.96 g, 79.67 mmol) and (E)-benzaldehyde oxime (5.7 g, 47.08 mmol), and the suspension was heated to 80° C. overnight. The solvent was evaporated in vacuo and the residue dissolved with ethyl acetate and water. The separated aqueous layer was acidified with 1M aqueous hydrochloric acid and extracted with ethyl acetate (3×) and once with dichloromethane. The combined organic phases were dried over sodium sulfate, filtered and concentrated. The residue was purified by flash chromatography on silica gel (gradient 0-10% methanol in dichloromethane) to afford methyl 3-ethylsulfanyl-5-hydroxy-pyridine-2-carboxylate (compound I33). LCMS (method 1): m/z 214 [M+H]⁺; retention time: 0.68 min.

### Step 2: Preparation of methyl 5-(2-amino-1,1-dimethyl-2-oxo-ethoxy)-3-ethylsulfanyl-pyridine-2-carboxylate (Compound I34)

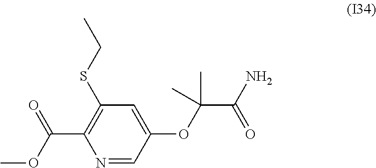 [SMILES: CCSc1cc(OC(C)(C)C(N)=O)cnc1C(=O)OC]

To a solution of methyl 3-ethylsulfanyl-5-hydroxy-pyridine-2-carboxylate (compound I33) (2.5 g, 11.72 mmol) in acetonitrile (59 ml) were added cesium carbonate (5.7 g, 17.49 mmol), and 2-bromo-2-methyl-propanamide (3.1 g, 18.67 mmol) after 5 minutes. The reaction mixture was stirred at room temperature overnight, poured into water and ethyl acetate. The separated aqueous layer was extracted with ethyl acetate (3×), the combined organic layers dried over sodium sulfate, filtered and evaporated to afford crude methyl 5-(2-amino-1,1-dimethyl-2-oxo-ethoxy)-3-ethylsulfanyl-pyridine-2-carboxylate (compound I34). This material was used without further purification into the next step.

LCMS (method 1): m/z 299 [M+H]⁺; retention time: 0.71 min.

### Step 3: Preparation of methyl 5-(1-cyano-1-methyl-ethoxy)-3-ethylsulfanyl-pyridine-2-carboxylate (Compound I35)

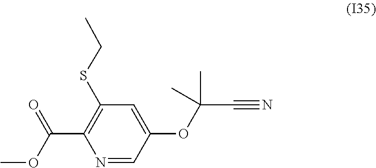 [SMILES: CCSc1cc(OC(C)(C)C#N)cnc1C(=O)OC]

To a mixture of crude methyl 5-(2-amino-1,1-dimethyl-2-oxo-ethoxy)-3-ethylsulfanyl-pyridine-2-carboxylate (compound I34 prepared above) (4.18 g, 14.0 mmol) and triethylamine (5.73 g, 7.89 ml, 56.0 mmol) in dichloromethane (140 ml) at 0° C. was added trifluoroacetic anhydride (8.92 g, 5.90 ml, 42.0 mmol) dropwise. The resulting suspension was stirred at room temperature for two hours. The reaction mixture was carefully quenched with methanol, then with an aqueous sodium hydrogen carbonate solution. The aqueous layer was extracted twice with dichloromethane, the combined organic layers dried over sodium sulfate, filtered and evaporated. The residue was purified by combiflash (0-45% gradient ethyl acetate in cyclohexane) to afford methyl 5-(1-cyano-1-methyl-ethoxy)-3-ethylsulfanyl-pyridine-2-carboxylate (compound I35). LCMS (method 1): m/z 281 [M+H]⁺; retention time: 0.90 min. ¹H NMR (400 MHz, CDCl₃) δ ppm 1.43 (t, J=7.40 Hz, 3H), 1.80 (s, 6H), 2.95 (q, J=7.40 Hz, 2H), 3.99 (s, 3H), 7.58 (d, J=2.32 Hz, 1H), 8.22 (d, J=2.32 Hz, 1H).

### Step 4: Preparation of 5-(1-cyano-1-methyl-ethoxy)-3-ethylsulfanyl-pyridine-2-carboxylic acid (Compound I36)

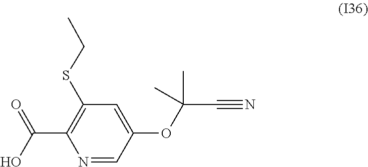 [SMILES: CCSc1cc(OC(C)(C)C#N)cnc1C(=O)O]

To a solution of methyl 5-(1-cyano-1-methyl-ethoxy)-3-ethylsulfanyl-pyridine-2-carboxylate (compound I35) (6.0 g, 21.41 mmol) in tetrahydrofuran (60 ml) were added lithium hydroxide hydrate (1.8 g, 42.81 mmol) and water (10 ml). The reaction mixture was stirred at room temperature until complete (TLC monitoring), then concentrated under reduced pressure. The residue was diluted with water (100 ml), acidified with a 2N aqueous hydrochloric acid solution and the aqueous phase extracted with ethyl acetate (3×100 ml). The combined organic layers were dried over sodium sulfate, filtered and concentrated in vacuo. The crude product was washed twice with n-pentane (50 ml), filtered and evaporated to dryness to give 5-(1-cyano-1-methyl-ethoxy)-3-ethylsulfanyl-pyridine-2-carboxylic acid (compound I36) as a solid. LCMS (method 4): m/z 267 [M+H]⁺ and m/z 265 [M−H]⁻; retention time: 0.82 min. ¹H NMR (400 MHz, DMSO-d6) δ ppm 1.27 (t, J=7.21 Hz, 3H), 1.78 (s, 6H), 2.97 (q, J=7.21 Hz, 2H), 7.58 (d, J=2.32 Hz, 1H), 8.24 (d, J=2.32 Hz, 1H).

### Step 5: Preparation of 5-(1-cyano-1-methyl-ethoxy)-3-ethylsulfanyl-pyridine-2-carbonyl chloride (Compound I32)

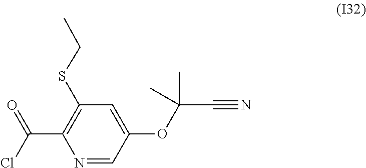 [SMILES: CCSc1cc(OC(C)(C)C#N)cnc1C(=O)Cl]

To a solution of 5-(1-cyano-1-methyl-ethoxy)-3-ethylsulfanyl-pyridine-2-carboxylic acid (compound I36) (771 mg, 2.90 mmol) and N,N-dimethylformamide (one drop) in tetrahydrofuran (19 ml) at 0-5° C. was added oxalyl chloride (0.328 ml, 3.76 mmol) and the mixture was stirred at room temperature for 2 hours. The solution was concentrated under reduced pressure, diluted twice with tetrahydrofuran and evaporated to dryness. LCMS data of an aliquot quenched with dimethylamine consistent with 5-(1-cyano-1-methyl-ethoxy)-3-ethylsulfanyl-N,N-dimethyl-pyridine-2-carboxamide (C₁₄H₁₉N₃O₂S, 293.38):

LCMS (method 1): m/z 294 [M+H]⁺; retention time: 0.83 min.

The following mixtures of the compounds of formula I with active ingredients are preferred (the abbreviation “TX” means “one compound selected from the group consisting of the compounds described in Tables A-1 through A-22, Tables B-1 through B-4 and Table P of the present invention”): an adjuvant selected from the group of substances consisting of petroleum oils (alternative name) (628)+TX;

an insect control active substance selected from Abamectin+TX, Acequinocyl+TX, Acetamiprid+TX, Acetoprole+TX, Acrinathrin+TX, Acynonapyr+TX, Afidopyropen+TX, Afoxolaner+TX, Alanycarb+TX, Allethrin+TX, Alpha-Cypermethrin+TX, Alphamethrin+TX, Amidoflumet+TX, Aminocarb+TX, Azocyclotin+TX, Bensultap+TX, Benzoximate+TX, Benzpyrimoxan+TX, Betacyfluthrin+TX, Beta-cypermethrin+TX, Bifenazate+TX, Bifenthrin+TX, Binapacryl+TX, Bioallethrin+TX, Bioallethrin S)-cyclopentylisomer+TX, Bioresmethrin+TX, Bistrifluron+TX, Broflanilide+TX, Brofluthrinate+TX, Bromophos-ethyl+TX, Buprofezine+TX, Butocarboxim+TX, Cadusafos+TX, Carbaryl+TX, Carbosulfan+TX, Cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2246757-58-2 (or 2249718-27-0)+TX, CAS number: 907187-07-9+TX, Chlorantraniliprole+TX, Chlordane+TX, Chlorfenapyr+TX, Chloroprallethrin+TX, Chromafenozide+TX, Clenpirin+TX, Cloethocarb+TX, Clothianidin+TX, 2-chlorophenyl N-methylcarbamate (CPMC)+TX, Cyanofenphos+TX, Cyantraniliprole+TX, Cyclaniliprole+TX, Cyclobutrifluram+TX, Cycloprothrin+TX, Cycloxaprid+TX, Cycloxaprid+TX, Cyenopyrafen+TX, Cyetpyrafen+TX, Cyflumetofen+TX, Cyfluthrin+TX, Cyhalodiamide+TX, Cyhalothrin+TX, Cypermethrin+TX, Cyphenothrin+TX, Cyproflanilide+TX, Cyromazine+TX, Deltamethrin+TX, Diafenthiuron+TX, Dialifos+TX, Dibrom+TX, Dicloromezotiaz+TX, Diflovidazine+TX, Diflubenzuron+TX, dimpropyridaz+TX, Dinactin+TX, Dinocap+TX, Dinotefuran+TX, Dioxabenzofos+TX, Emamectin (or Emamectin Benzoate)+TX, Empenthrin+TX, Epsilon-momfluorothrin+TX, Epsilon-metofluthrin+TX, Esfenvalerate+TX, Ethion+TX, Ethiprole+TX, Etofenprox+TX, Etoxazole+TX, Famphur+TX, Fenazaquin+TX, Fenfluthrin+TX, Fenitrothion+TX, Fenobucarb+TX, Fenothiocarb+TX, Fenoxycarb+TX, Fenpropathrin+TX, Fenpyroxymate+TX, Fensulfothion+TX, Fenthion+TX, Fentinacetate+TX, Fenvalerate+TX, Fipronil+TX, Flometoquin+TX, Flonicamid+TX, Fluacrypyrim+TX, Fluazaindolizine+TX, Fluazuron+TX, Flubendiamide+TX, Flubenzimine+TX, Flucitrinate+TX, Flucycloxuron+TX, Flucythrinate+TX, Fluensulfone+TX, Flufenerim+TX, Flufenprox+TX, Flufiprole+TX, Fluhexafon+TX, Flumethrin+TX, Fluopyram+TX, Flupentiofenox+TX, Flupyradifurone+TX, Flupyrimin+TX, Fluralaner+TX, Fluvalinate+TX, Fluxametamide+TX, Fosthiazate+TX, Gamma-Cyhalothrin+TX, Gossyplure™+TX, Guadipyr+TX, Halofenozide+TX, Halofenozide+TX, Halfenprox+TX, Heptafluthrin+TX, Hexythiazox+TX, Hydramethylnon+TX, Imicyafos+TX, Imidacloprid+TX, Imiprothrin+TX, Indoxacarb+TX, Iodomethane+TX, Iprodione+TX, Isocycloseram+TX, Isothioate+TX, Ivermectin+TX, Kappa-bifenthrin+TX, Kappa-tefluthrin+TX, Lambda-Cyhalothrin+TX, Lepimectin+TX, Lufenuron+TX, Metaflumizone+TX, Metaldehyde+TX, Metam+TX, Methomyl+TX, Methoxyfenozide+TX, Metofluthrin+TX, Metolcarb+TX, Mexacarbate+TX, Milbemectin+TX, Momfluorothrin+TX, Niclosamide+TX, Nicofluprole+TX; Nitenpyram+TX, Nithiazine+TX, Omethoate+TX, Oxamyl+TX, Oxazosulfyl+TX, Parathion-ethyl+TX, Permethrin+TX, Phenothrin+TX, Phosphocarb+TX, Piperonylbutoxide+TX, Pirimicarb+TX, Pirimiphos-ethyl+TX, Pirimiphos-methyl+TX, Polyhedrosis virus+TX, Prallethrin+TX, Profenofos+TX, Profenofos+TX, Profluthrin+TX, Propargite+TX, Propetamphos+TX, Propoxur+TX, Prothiophos+TX, Protrifenbute+TX, Pyflubumide+TX, Pymetrozine+TX, Pyraclofos+TX, Pyrafluprole+TX, Pyridaben+TX, Pyridalyl+TX, Pyrifluquinazon+TX, Pyrimidifen+TX, Pyriminostrobin+TX, Pyriprole+TX, Pyriproxyfen+TX, Resmethrin+TX, Sarolaner+TX, Selamectin+TX, Silafluofen+TX, Spinetoram+TX, Spinosad+TX, Spirodiclofen+TX, Spiromesifen+TX, Spiropidion+TX, Spirotetramat+TX, Sulfoxaflor+TX, Tebufenozide+TX, Tebufenpyrad+TX, Tebupirimiphos+TX, Tefluthrin+TX, Temephos+TX, Tetrachlorantraniliprole+TX, Tetradiphon+TX, Tetramethrin+TX, Tetramethylfluthrin+TX, Tetranactin+TX, Tetraniliprole+TX, Theta-cypermethrin+TX, Thiacloprid+TX, Thiamethoxam+TX, Thiocyclam+TX, Thiodicarb+TX, Thiofanox+TX, Thiometon+TX, Thiosultap+TX, Tioxazafen+TX, Tolfenpyrad+TX, Toxaphene+TX, Tralomethrin+TX, Transfluthrin+TX, Triazamate+TX, Triazophos+TX, Trichlorfon+TX, Trichloronate+TX, Trichlorphon+TX, Triflumezopyrim+TX, Tyclopyrazoflor+TX, Zeta-Cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, Azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.;

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;

an anthelmintic selected from the group of substances consisting of abamectin (1)+TX, crufomate (1011)+TX, Cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;

an avicide selected from the group of substances consisting of chloralose (127)+TX, endrin (1122)+TX, fenthion (346)+TX, pyridin-4-amine (IUPAC name) (23) and strychnine (745)+TX;

a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;

a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;

a soil sterilant selected from the group of substances consisting of iodomethane (IUPAC name) (542) and methyl bromide (537)+TX;

a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX;

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, Cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;

a nitrification inhibitor selected from the group of substances consisting of potassium ethylxanthate [CCN] and nitrapyrin (580)+TX;

a plant activator selected from the group of substances consisting of acibenzolar (6)+TX, acibenzolar-S-methyl (6)+TX, probenazole (658) and Reynoutria sachalinensis extract (alternative name) (720)+TX;

a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;

a virucide selected from the group of substances consisting of imanin (alternative name) [CCN] and ribavirin (alternative name) [CCN]+TX;

a wound protectant selected from the group of substances consisting of mercuric oxide (512)+TX, octhilinone (590) and thiophanate-methyl (802)+TX;

a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, -metalaxyl-+TX, Rmetalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1, 1-dimethylethyl)-α-[4′-(trifluoromethoxy) [1, 1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179);

3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®);

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate;

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline I®)+TX, Orius laevigatus (Thripor-L®+TX, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator;

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX; and

a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables A-1 through A-22, Tables B-1 through B-4 and Table P with active ingredients described above comprises a compound selected from Tables A-1 through A-22, Tables B-1 through B-4 and Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables A-1 through A-22, Tables B-1 through B-4 and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables A-1 through A-22, Tables B-1 through B-4 and Table P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I including those selected from Tables A-1 through A-22, Tables B-1 through B-4 and Table P. Further, it is hereby made available, a composition comprising a plant propagation material treated with a compound of formula I including those selected from Tables A-1 through A-22, Tables B-1 through B-4 and Table P.

Seed treatment comprises all suitable seed treatment techniques known in the art, such as seed dressing, seed coating, seed dusting, seed soaking and seed pelleting. The seed treatment application of the compound formula I (including those selected from Tables A-1 through A-22, Tables B-1 through B-4 and Table P) can be carried out by any known methods, such as spraying or by dusting the seeds before sowing or during the sowing/planting of the seeds.

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

### Example B1: Activity Against Bemisia tabaci (Cotton White Fly)

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P1, P2, P3, P4, P5, P6, P9, P10, P11, P12, P13, P14, P15.

### Example B2: Activity Against Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P1, P2, P3, P4, P5, P6, P7, P8, P9, P10, P11, P12, P13, P14, P15, P16.

### Example B3: Activity Against Euschistus heros (Neotropical Brown Stink Bug)

Soybean leaf on agar in 24-well microtiter plates were sprayed with aqueous test solutions prepared from 10′000 ppm DMSO stock solutions. After drying the leaf were infested with N-2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P3, P4, P5, P6, P7, P8, P10, P11, P12, P13, P14, P15, P16.

### Example B4: Activity Against Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm: P3, P6, P7, P8, P9, P10, P12, P13, P14.

### Example B5: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm: P1, P3, P13, P14, P15, P16.

### Example B6: Activity Against Myzus persicae (Green Peach Aphid)

Sunflower leaf discs were placed on agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P1, P2, P3, P4, P5, P6, P8, P9, P10, P11, P12, P13, P14, P15, P16.

### Example B7: Activity Against Myzus persicae (Green Peach Aphid)

Roots of pea seedlings infested with an aphid population of mixed ages were placed directly in the aqueous test solutions prepared from 10′000 DMSO stock solutions. The samples were assessed for mortality 6 days after placing seedlings in test solutions.

The following compounds resulted in at least 80% mortality at a test rate of 24 ppm: P2, P5, P11.

### Example B8: Activity Against Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: P2, P4, P5, P6, P7, P8, P9, P10, P11, P12.

### Example B9: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P1, P2, P3, P4, P5, P6, P7, P8, P9, P10, P11, P12, P13, P14, P15, P16.

### Example B10: Activity Against Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P1, P4, P5, P8, P10.

### Example B11: Activity Against Nilaparvata lugens (Brown Plant Hopper) Larvicide, Feeding/Contact

Rice plants were treated with the diluted test solutions in a spray chamber. After drying plants were infested with ˜20 N3 nymphs. 7 days after the treatment samples were assessed for mortality and growth regulation.

The following compounds resulted in at least 80% mortality at an application rate of 50 ppm: P1, P2, P9, P10, P11.

### Example B12: Activity Against Nilaparvata lugens (Brown Plant Hopper) Larvicide, Systemic into Water

Rice plants cultivated in a nutritive solution were treated with the diluted test solutions into nourishing cultivation system. 1 day after application plants were infested with ˜20 N3 nymphs. 7 days after infestation samples were assessed for mortality and growth regulation.

The following compounds resulted in at least 80% mortality at an application rate of 12.5 ppm: P1, P2, P4, P5, P9, P10, P11.

### Example B13: Activity Against Heterodera schachtii Juvenile Mobility In Vitro Profiling in 96 Well Plate

Test solutions are prepared from 10′000 ppm DMSO stock solutions with a TECAN robot to achieve 20 μL of 500, 100, 50, 25, 12.5 and 6.25 ppm. For each concentration three replicates are produced. Per well, 80 μL nematode solution is added containing 100 to 150 freshly harvested second stage juveniles of Heterodera schachtii. The plates are covered and stored at room temperature in the dark and incubated for 48 h. Mobility of the exposed juveniles in a treated well is measured using an imaging tool and compared to an average of 12 untreated replicates.

The following compounds achieved at least 60% control at 100 ppm after 48 h: P1, P4, P6, P8, P9, P10.

### Example B14: Activity Against Melodoigyne incognita Juvenile Mobility In Vitro Profiling in 96 Well Plate

Test solutions are prepared from 10′000 ppm DMSO stock solutions with a TECAN robot to achieve 20 μL of 1000, 200, 100, 50, 25 and 12.5 ppm. For each concentration three replicates are produced. Per well, 80 μL nematode solution is added containing 100 to 150 freshly harvested second stage juveniles of Melodoigyne incognita. The plates are covered and stored at room temperature in the dark and incubated for 48 h. Mobility of the exposed juveniles in a treated well is measured using an imaging tool and compared to an average of 12 untreated replicates

The following compounds achieved at least 60% control at 200 ppm after 48 h: P4, P8.

### Example B15: Activity Against Carpocapsa (Cydia) pomonella (Codling Moth), Larvicide, Feeding/Contact

Diet cubes coated with paraffin were sprayed with diluted test solutions in an application chamber. After drying off the treated cubes (10 replicates) were infested with 1 L1 larvae. Samples were incubated at 26-27° C. and checked 14 days after infestation for mortality and growth inhibition.

The following compounds gave an effect of at least 80% in at least one of the two categories (mortality or growth inhibition) at an application rate of 12.5 ppm: P4, P5, P11, P15, P16.

1. A compound of formula (I) 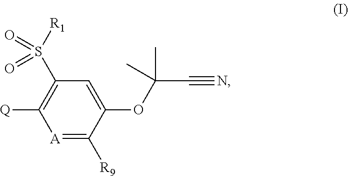
wherein
A is CH or N;
R₁ is C₁-C₄ alkyl or C₃-C₆cycloalkyl-C₁-C₄alkyl;
R₉ is hydrogen or C₁-C₄alkyl;
Q is a radical selected from the group consisting of formula Q₁ to Q₇ 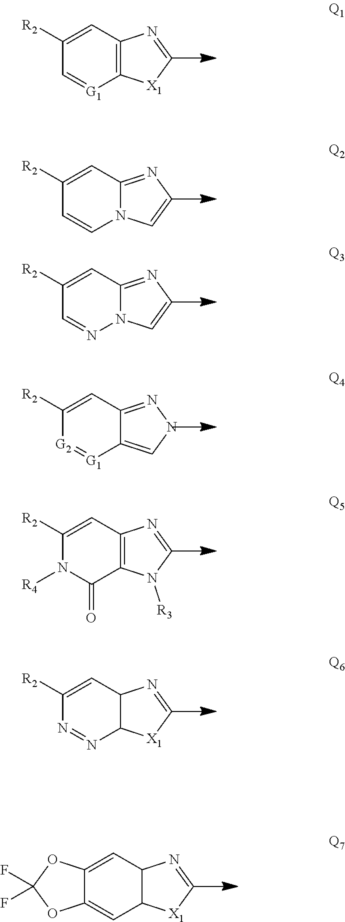
wherein the arrow denotes the point of attachment to the ring incorporating the radical A;
and wherein
X₁ is O, S or NR₃;
R₃ is C₁-C₄alkyl;
R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;
G₁ and G₂ are, independently from each other, N or CH;
R₄ is C₁-C₄alkyl, C₁-C₄haloalkyl, C₃-C₆cycloalkyl or C₁-C₄alkoxy; or
an agrochemically acceptable salt, stereoisomer, enantiomer, tautomer or N-oxide of a compound of formula I.

2. A compound of formula I according to claim 1, represented by the compounds of formula I-1 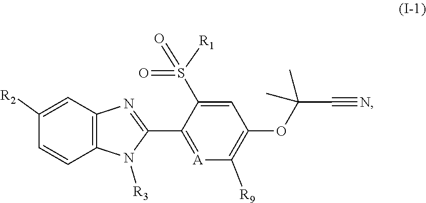
wherein R₁, R₂, R₃, R₉, and A are as defined under formula I in claim 1; or an agrochemically acceptable salt, stereoisomer, enantiomer, tautomer or N-oxide of formula I-1.

3. A compound of formula I according to claim 1, represented by the compounds of formula I-2 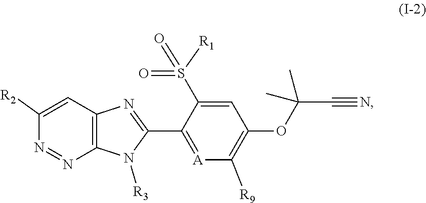
wherein R₁, R₂, R₃, R₉, and A are as defined under formula I in claim 1; or an agrochemically acceptable salt, stereoisomer, enantiomer, tautomer or N-oxide of formula I-2.

4. A compound of formula I according to claim 1, represented by the compounds of formula I-3 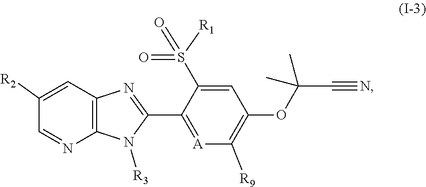
wherein R₁, R₂, R₃, R₉, and A are as defined under formula I in claim 1; or an agrochemically acceptable salt, stereoisomer, enantiomer, tautomer or N-oxide of formula I-3.

5. A compound of formula I according to claim 1, represented by the compounds of formula I-4 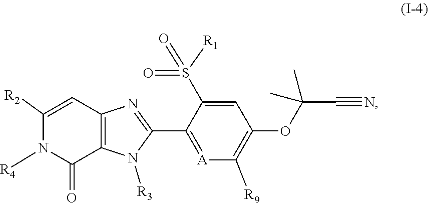
wherein R₁, R₂, R₃, R₄, R₉, and A are as defined under formula I in claim 1; or an agrochemically acceptable salt, stereoisomer, enantiomer, tautomer or N-oxide of formula I-4.

6. A compound according to claim 1 wherein R₄ is ethyl, methoxy or cyclopropyl.

7. A compound according to claim 1 wherein R₃ is methyl.

8. A compound of formula I according to claim 1, represented by the compounds of formula I-5 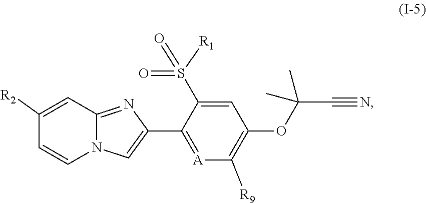
wherein R₁, R₂, R₉, and A are as defined under formula I in claim 1; or an agrochemically acceptable salt, stereoisomer, enantiomer, tautomer or N-oxide of formula I-5.

9. A compound of formula I according to claim 1, represented by the compounds of formula I-6 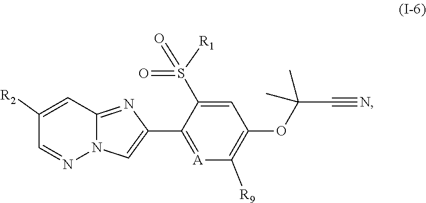
wherein R₁, R₂, R₉, and A are as defined under formula I in claim 1; or an agrochemically acceptable salt, stereoisomer, enantiomer, tautomer or N-oxide of formula I-6.

10. A compound of formula I according to claim 1, represented by the compounds of formula I-7 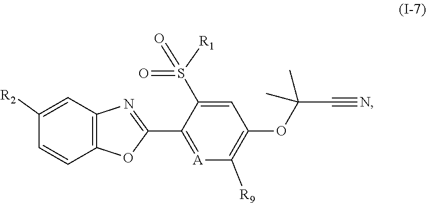
wherein R₁, R₂, R₉, and A are as defined under formula I in claim 1; or an agrochemically acceptable salt, stereoisomer, enantiomer, tautomer or N-oxide of formula I-7.

11. A compound of formula I according to claim 1, represented by the compounds of formula I-8 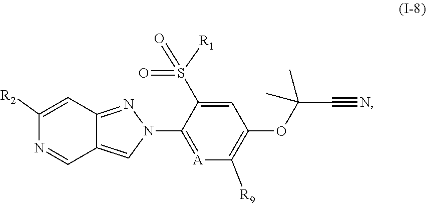
wherein R₁, R₂, R₉, and A are as defined under formula I in claim 1; or an agrochemically acceptable salt, stereoisomer, enantiomer, tautomer or N-oxide of formula I-8.

12. A compound of formula I according to claim 1, represented by the compounds of formula I-9 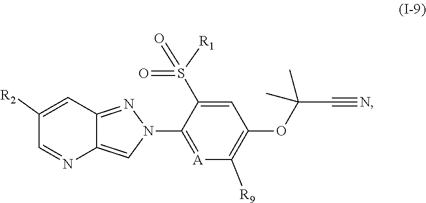
wherein R₁, R₂, R₉, and A are as defined under formula I in claim 1; or an agrochemically acceptable salt, stereoisomer, enantiomer, tautomer or N-oxide of formula I-9.

13. A compound according to claim 1 wherein R₂ is trifluoromethyl, pentafluoroethyl, trifluoromethylsulfanyl, trifluoromethylsulfinyl or trifluoromethylsulfonyl; preferably is R₂ is trifluoromethyl, trifluoromethylsulfonyl or trifluoromethylsulfanyl.

14. A compound of formula I according to claim 1, represented by the compounds of formula I-10 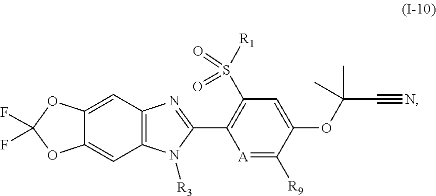
wherein R₁, R₃, R₉, and A are as defined under formula I in claim 1; or
an agrochemically acceptable salt, stereoisomer, enantiomer, tautomer or N-oxide of formula I-10.

15. A compound of formula I according to claim 1, represented by the compounds of formula I-11 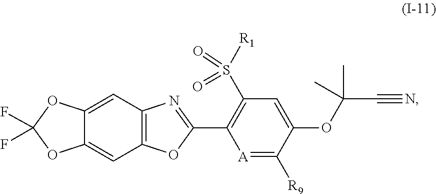
wherein R₁, R₉, and A are as defined under formula I above; or an agrochemically acceptable salt, stereoisomer, enantiomer, tautomer or N-oxide of formula I-11.

16. A compound according to claim 1 wherein R₁ is ethyl or —CH₂cyclopropyl; preferably R₁ is ethyl.

17. A compound according to a claim 1 wherein R₉ is hydrogen, methyl or ethyl; preferably; R₉ is hydrogen.

18. A compound of formula I according to claim 1 selected from the group consisting of:
2-[[5-(cyclopropylmethylsulfonyl)-6-[7-methyl-3-(trifluoromethyl)imidazo[4,5-c]pyridazin-6-yl]-3-pyridyl]oxy]-2-methyl-propanenitrile (compound P1);
2-[[5-ethylsulfonyl-6-[7-methyl-3-(trifluoromethyl)imidazo[4,5-c]pyridazin-6-yl]-3-pyridyl]oxy]-2-methyl-propanenitrile (compound P2);
2-[[5-ethylsulfonyl-6-[3-methyl-6-(trifluoromethyl)imidazo[4,5-b]pyridin-2-yl]-3-pyridyl]oxy]-2-methyl-propanenitrile (compound P3);
2-[[5-ethylsulfonyl-2-methyl-6-[3-methyl-6-(trifluoromethyl)imidazo[4,5-b]pyridin-2-yl]-3-pyridyl]oxy]-2-methyl-propanenitrile (compound P4);
2-[[5-ethylsulfonyl-6-[5-methoxy-3-methyl-4-oxo-6-(trifluoromethyl)imidazo[4,5-c]pyridin-2-yl]-3-pyridyl]oxy]-2-methyl-propanenitrile (compound P5);
2-[[6-[5-ethyl-3-methyl-4-oxo-6-(trifluoromethyl)imidazo[4,5-c]pyridin-2-yl]-5-ethylsulfonyl-3-pyridyl]oxy]-2-methyl-propanenitrile (compound P6);
2-[[6-[5-cyclopropyl-3-methyl-4-oxo-6-(trifluoromethyl)imidazo[4,5-c]pyridin-2-yl]-5-ethylsulfonyl-3-pyridyl]oxy]-2-methyl-propanenitrile (compound P7);
2-[[5-ethylsulfonyl-6-[7-(trifluoromethyl)imidazo[1,2-a]pyridin-2-yl]-3-pyridyl]oxy]-2-methyl-propanenitrile (compound P8);
2-[[5-ethylsulfonyl-6-[7-(trifluoromethyl)imidazo[1,2-b]pyridazin-2-yl]-3-pyridyl]oxy]-2-methyl-propanenitrile (compound P9);
2-[3-ethylsulfonyl-4-[6-(trifluoromethyl)pyrazolo[4,3-c]pyridin-2-yl]phenoxy]-2-methyl-propanenitrile (compound P10);
2-[[5-ethylsulfonyl-2-methyl-6-[7-methyl-3-(trifluoromethyl)imidazo[4,5-c]pyridazin-6-yl]-3-pyridyl]oxy]-2-methyl-propanenitrile (compound P11);
2-[3-ethylsulfonyl-4-[7-methyl-3-(trifluoromethyl)imidazo[4,5-c]pyridazin-6-yl]phenoxy]-2-methyl-propanenitrile (compound P12);
2-[[5-ethylsulfonyl-6-[1-methyl-5-(trifluoromethylsulfanyl)benzimidazol-2-yl]-3-pyridyl]oxy]-2-methyl-propanenitrile (compound P13);
2-[[5-ethylsulfonyl-6-[1-methyl-5-(trifluoromethylsulfonyl)benzimidazol-2-yl]-3-pyridyl]oxy]-2-methyl-propanenitrile (compound P14);
2-[[6-(2,2-difluoro-7-methyl-[1,3]dioxolo[4,5-f]benzimidazol-6-yl)-5-ethylsulfonyl-3-pyridyl]oxy]-2-methyl-propanenitrile (compound P15); and
2-[[6-(2,2-difluoro-[1,3]dioxolo[4,5-f][1,3]benzoxazol-6-yl)-5-ethylsulfonyl-3-pyridyl]oxy]-2-methyl-propanenitrile (compound P16).

19. A pesticidal composition, which comprises at least one compound of formula I as defined in claim 1 or, where appropriate, a tautomer thereof, in each case in free form or in agrochemically utilizable salt form, as active ingredient and at least one auxiliary.

20. A method for controlling pests, which comprises applying to a pest, to a locus of a pest, or to a plant susceptible to attack by a pest, a pesticidally effective amount of a compound of formula I as defined in claim 1.

21. A method for the protection of plant propagation material from the attack by pests, which comprises treating the propagation material or the site, where the propagation material is planted, with a composition according to claim 19.
